SEQUENCE LISTING (22 sequences)

SEQ ID NO: 1 - DNA, 1773 bp, Arabidopsis thaliana
atgtctcttcctgaaactaaatctgatgatatccttcttgatgcttgggacttccaaggccgtcccgccgatcgctcaaaaaccggcggctgggccagcgccgccatgattctttgtattgaggccgtggagaggctgacgacgttaggtatcggagttaatctggtgacgtatttgacgggaactatgcatttaggcaatgcaactgcggctaacaccgttaccaatttcctcggaacttctttcatgctctgtctcctcggtggcttcatcgccgatacctttctcggcaggtacctaacgattgctatattcgccgcaatccaagccacgggtgtttcaatcttaactctatcaacaatcataccgggacttcgaccaccaagatgcaatccaacaacgtcgtctcactgcgaacaagcaagtggaatacaactgacggtcctatacttagccttatacctcaccgctctaggaacgggaggcgtgaaggctagtgtctcgggtttcgggtcggaccaattcgatgagaccgaaccaaaagaacgatcgaaaatgacatatttcttcaaccgtttcttcttttgtatcaacgttggctctcttttagctgtgacggtccttgtctacgtacaagacgatgttggacgcaaatggggctatggaatttgcgcgtttgcgatcgtgcttgcactcagcgttttcttggccggaacaaaccgctaccgtttcaagaagttgatcggtagcccgatgacgcaggttgctgcggttatcgtggcggcgtggaggaataggaagctcgagctgccggcagatccgtcctatctctacgatgtggatgatattattgcggcggaaggttcgatgaagggtaaacaaaagctgccacacactgaacaattccgttcattagataaggcagcaataagggatcaggaagcgggagttacctcgaatgtattcaacaagtggacactctcaacactaacagatgttgaggaagtgaaacaaatcgtgcgaatgttaccaatttgggcaacatgcatcctcttctggaccgtccacgctcaattaacgacattatcagtcgcacaatccgagacattggaccgttccatcgggagcttcgagatccctccagcatcgatggcagtcttctacgtcggtggcctcctcctaaccaccgccgtctatgaccgcgtcgccattcgtctatgcaaaaagctattcaactacccccatggtctaagaccgcttcaacggatcggtttggggcttttcttcggatcaatggctatggctgtggctgctttggtcgagctcaaacgtcttagaactgcacacgctcatggtccaacagtcaaaacgcttcctctagggttttatctactcatcccacaatatcttattgtcggtatcggcgaagcgttaatctacacaggacagttagatttcttcttgagagagtgccctaaaggtatgaaagggatgagcacgggtctattgttgagcacattggcattaggctttttcttcagctcggttctcgtgacaatcgtcgagaaattcaccgggaaagctcatccatggattgccgatgatctcaacaagggccgtctttacaatttctactggcttgtggccgtacttgttgccttgaacttcctcattttcctagttttctccaagtggtacgtttacaaggaaaaaagactagctgaggtggggattgagttggatgatgagccgagtattccaatgggtcattga

SEQ ID NO: 2 - protein, 590 aa, Arabidopsis thaliana
MSLPETKSDDILLDAWDFQGRPADRSKTGGWASAAMILCIEAVERLTTLGIGVNLVTYLTGTMHLGNATAANTVTNFLGTSFMLCLLGGFIADTFLGRYLTIAIFAAIQATGVSILTLSTIIPGLRPPRCNPTTSSHCEQASGIQLTVLYLALYLTALGTGGVKASVSGFGSDQFDETEPKERSKMTYFFNRFFFCINVGSLLAVTVLVYVQDDVGRKWGYGICAFAIVLALSVFLAGTNRYRFKKLIGSPMTQVAAVIVAAWRNRKLELPADPSYLYDVDDIIAAEGSMKGKQKLPHTEQFRSLDKAAIRDQEAGVTSNVFNKWTLSTLTDVEEVKQIVRMLPIWATCILFWTVHAQLTTLSVAQSETLDRSIGSFEIPPASMAVFYVGGLLLTTAVYDRVAIRLCKKLFNYPHGLRPLQRIGLGLFFGSMAMAVAALVELKRLRTAHAHGPTVKTLPLGFYLLIPQYLIVGIGEALIYTGQLDFFLRECPKGMKGMSTGLLLSTLALGFFFSSVLVTIVEKFTGKAHPWIADDLNKGRLYNFYWLVAVLVALNFLIFLVFSKWYVYKEKRLAEVGIELDDEPSIPMGH

SEQ ID NO: 3 - protein, 570 aa, Arabidopsis thaliana
MEEKDVYTQDGTVDIHKNPANKEKTGNWKACRFILGNECCERLAYYGMGTNLVNYLESRLNQGNATAANNVTNWSGTCYITPLIGAFIADAYLGRYWTIATFVFIYVSGMTLLTLSASVPGLKPGNCNADTCHPNSSQTAVFFVALYMIALGTGGIKPCVSSFGADQFDENDENEKIKKSSFFNWFYFSINVGALIAATVLVWIQMNVGWGWGFGVPTVAMVIAVCFFFFGSRFYRLQRPGGSPLTRIFQVIVAAFRKISVKVPEDKSLLFETADDESNIKGSRKLVHTDNLKFFDKAAVESQSDSIKDGEVNPWRLCSVTQVEELKSIITLLPVWATGIVFATVYSQMSTMFVLQGNTMDQHMGKNFEIPSASLSLFDTVSVLFWTPVYDQFIIPLARKFTRNERGFTQLQRMGIGLVVSIFAMITAGVLEVVRLDYVKTHNAYDQKQIHMSIFWQIPQYLLIGCAEVFTFIGQLEFFYDQAPDAMRSLCSALSLTTVALGNYLSTVLVTVVMKITKKNGKPGWIPDNLNRGHLDYFFYLLATLSFLNFLVYLWISKRYKYKKAVGRAH

SEQ ID NO: 4 - protein, 585 aa, Arabidopsis thaliana
MGSIEEEARPLIEEGLILQEVKLYAEDGSVDFNGNPPLKEKTGNWKACPFILGNECCERLAYYGIAGNLITYLTTKLHQGNVSAATNVTTWQGTCYLTPLIGAVLADAYWGRYWTIACFSGIYFIGMSALTLSASVPALKPAECIGDFCPSATPAQYAMFFGGLYLIALGTGGIKPCVSSFGADQFDDTDSRERVRKASFFNWFYFSINIGALVSSSLLVWIQENRGWGLGFGIPTVFMGLAIASFFFGTPLYRFQKPGGSPITRISQVVVASFRKSSVKVPEDATLLYETQDKNSAIAGSRKIEHTDDCQYLDKAAVISEEESKSGDYSNSWRLCTVTQVEELKILIRMFPIWASGIIFSAVYAQMSTMFVQQGRAMNCKIGSFQLPPAALGTFDTASVIIWVPLYDRFIVPLARKFTGVDKGFTEIQRMGIGLFVSVLCMAAAAIVEIIRLHMANDLGLVESGAPVPISVLWQIPQYFILGAAEVFYFIGQLEFFYDQSPDAMRSLCSALALLTNALGNYLSSLILTLVTYFTTRNGQEGWISDNLNSGHLDYFFWLLAGLSLVNMAVYFFSAARYKQKKASS

SEQ ID NO: 5 - protein, 545 aa, Arabidopsis thaliana
MASIDEERSLLEVEESLIQEEVKLYAEDGSIDIHGNPPLKQTTGNWKACPFIFANECCERLAYYGIAKNLITYFTNELHETNVSAARHVMTWQGTCYITPLIGALIADAYWGRYWTIACFSAIYFTGMVALTLSASVPGLKPAECIGSLCPPATMVQSTVLFSGLYLIALGTGGIKPCVSSFGADQFDKTDPSERVRKASFFNWFYFTINIGAFVSSTVLVWIQENYGWELGFLIPTVFMGLATMSFFFGTPLYRFQKPRGSPITSVCQVLVAAYRKSNLKVPEDSTDEGDANTNPWKLCTVTQVEEVKILLRLVPIWASGIIFSVLHSQIYTLFVQQGRCMKRTIGLFEIPPATLGMFDTASVLISVPIYDRVIVPLVRRFTGLAKGFTELQRMGIGLFVSVLSLTFAAIVETVRLQLARDLDLVESGDIVPLNIFWQIPQYFLMGTAGVFFFVGRIEFFYEQSPDSMRSLCSAWALLTTTLGNYLSSLIITLVAYLSGKDCWIPSDNINNGHLDYFFWLLVSLGSVNIPVFVFFSVKYTHMKV

SEQ ID NO: 6 - protein, 570 aa, Arabidopsis thaliana
MEDDKDIYTKDGTLDIHKKPANKNKTGTWKACRFILGTECCERLAYYGMSTNLINYLEKQMNMENVSASKSVSNWSGTCYATPLIGAFIADAYLGRYWTIASFVVIYIAGMTLLTISASVPGLTPTCSGETCHATAGQTAITFIALYLIALGTGGIKPCVSSFGADQFDDTDEKEKESKSSFFNWFYFVINVGAMIASSVLVWIQMNVGWGWGLGVPTVAMAIAVVFFFAGSNFYRLQKPGGSPLTRMLQVIVASCRKSKVKIPEDESLLYENQDAESSIIGSRKLEHTKILTFFDKAAVETESDNKGAAKSSSWKLCTVTQVEELKALIRLLPIWATGIVFASVYSQMGTVFVLQGNTLDQHMGPNFKIPSASLSLFDTLSVLFWAPVYDKLIVPFARKYTGHERGFTQLQRIGIGLVISIFSMVSAGILEVARLNYVQTHNLYNEETIPMTIFWQVPQYFLVGCAEVFTFIGQLEFFYDQAPDAMRSLCSALSLTAIAFGNYLSTFLVTLVTKVTRSGGRPGWIAKNLNNGHLDYFFWLLAGLSFLNFLVYLWIAKWYTYKKTTGHAL

SEQ ID NO: 7 - DNA, 1713 bp, Arabidopsis thaliana
atggaagaaaaagatgtgtatacgcaagatggaactgttgatattcacaaaaatcctgcaaacaaggagaaaaccggaaattggaaagcttgccgcttcattctcggaaatgagtgctgtgaaagattggcctactatggcatgggcactaaccttgtgaattatcttgagagccgtctgaatcaaggcaatgctacggctgcaaataacgtcacgaattggtctggaacatgttatataactcctttgattggagcctttatagctgatgcttaccttggacgatattggactattgcaacttttgttttcatctatgtctccggtatgactcttttgacattatcagcttcagttcctggacttaaaccaggtaactgcaatgctgatacttgtcatccaaattctagtcagactgctgttttctttgtcgcgctttatatgattgctcttggaactggcggtataaagccgtgtgtttcgtcctttggagctgatcagtttgatgagaatgatgagaatgagaagatcaagaaaagttctttcttcaactggttttacttctccattaatgttggagctctcattgctgcaactgttctcgtctggatacaaatgaatgttggttggggatggggtttcggtgttccaacagtcgcgatggttatcgcggtttgctttttcttcttcggaagccgtttttacagacttcagagacctggagggagtccacttactaggatctttcaggttatagtagcggcttttcggaagataagtgttaaggttccagaggacaagtctctgctctttgaaactgcagatgatgagagtaacatcaaaggtagccggaaacttgtgcacacagataacttaaagttttttgacaaggcagcggttgagagtcaatctgatagcatcaaagacggggaagtcaatccatggagactatgttctgttactcaagttgaagaacttaagtcaataatcacacttcttccagtttgggccacaggaatagtcttcgccacagtgtacagccaaatgagcacaatgtttgtgttacaaggaaacacaatggaccaacacatgggaaaaaactttgaaatcccatcagcttcactctcacttttcgacactgtcagtgtactcttctggactcctgtctatgaccagttcattatcccgctggcaagaaagttcacacgcaatgaacgaggcttcactcagcttcaacgtatgggtataggtcttgtggtctccatctttgccatgatcactgcaggagtcttggaggttgtcaggcttgattatgtcaaaactcacaatgcatatgaccaaaaacagatccatatgtcgatattctggcagataccgcagtatttacttatcggttgtgcagaagttttcacctttataggtcagcttgagtttttctatgatcaggctcctgatgccatgagaagtctctgctctgctttgtcgttgaccacggttgcgttggggaactatttgagcacagttcttgtgacggttgtgatgaagataacgaagaagaacggtaaaccgggttggataccggataacttgaaccgaggccatcttgattactttttctacttgttggcaactctcagtttcctcaacttcttagtgtacctctggatttcaaaacgctacaaatacaagaaagctgttggtcgagcacattga

SEQ ID NO: 8 - DNA, 1758 bp, Arabidopsis thaliana
atgggttccatcgaagaagaagcaagacctctcatcgaagaaggtttaattttacaggaagtgaaattgtatgctgaagatggttcagtggactttaatggaaacccaccattgaaggagaaaacaggaaactggaaagcttgtccttttattcttggtaatgaatgttgtgagaggctagcttactatggtattgctgggaatttaatcacttacctcaccactaagcttcaccaaggaaatgtttctgctgctacaaacgttaccacatggcaagggacttgttatctcactcctctcattggagctgttctggctgatgcttactggggacgttactggaccatcgcttgtttctccgggatttatttcatcgggatgtctgcgttaactctttcagcttcagttccggcattgaagccagcggaatgtattggtgacttttgtccatctgcaacgccagctcagtatgcgatgttctttggtgggctttacctgatcgctcttggaactggaggtatcaaaccgtgtgtctcatccttcggtgccgatcagtttgatgacacggactctcgggaacgagttagaaaagcttcgttctttaactggttttacttctccatcaatattggagcacttgtgtcatctagtcttctagtttggattcaagagaatcgcgggtggggtttagggtttgggataccaacagtgttcatgggactagccattgcaagtttcttctttggcacacctctttataggtttcagaaacctggaggaagccctataactcggatttcccaagtcgtggttgcttcgttccggaaatcgtctgtcaaagtccctgaagacgccacacttctgtatgaaactcaagacaagaactctgctattgctggaagtagaaaaatcgagcataccgatgattgccagtatcttgacaaagccgctgttatctcagaagaagaatcgaaatccggagattattccaactcgtggagactatgcacggttacgcaagtcgaagaactcaagattctgatccgaatgttcccaatctgggcttctggtatcattttctcagctgtatacgcacaaatgtccacaatgtttgttcaacaaggccgagccatgaactgcaaaattggatcattccagcttcctcctgcagcactcgggacattcgacacagcaagcgtcatcatctgggtgccgctctacgaccggttcatcgttcccttagcaagaaagttcacaggagtagacaaaggattcactgagatacaaagaatgggaattggtctgtttgtctctgttctctgtatggcagctgcagctatcgtcgaaatcatccgtctccatatggccaacgatcttggattagtcgagtcaggagccccagttcccatatccgtcttgtggcagattccacagtacttcattctcggtgcagccgaagtattctacttcatcggtcagctcgagttcttctacgaccaatctccagatgcaatgagaagcttgtgcagtgccttggctcttttgaccaatgcacttggtaactacttgagctcgttgatcctcacgctcgtgacttattttacaacaagaaatgggcaagaaggttggatttcggataatctcaattcaggtcatctcgattacttcttctggctcttggctggtcttagccttgtgaacatggcggtttacttcttctctgctgctaggtataagcaaaagaaagcttcgtcgtag

SEQ ID NO: 9 - DNA, 1638 bp, Arabidopsis thaliana
atggcttccattgatgaagaaaggtcacttcttgaagttgaagaatctcttatacaggaagaagtaaaattatatgctgaagatggttcaatagatattcatggaaacccaccattgaagcagacaacaggaaactggaaagcttgtccattcatttttgcaaacgaatgctgcgaacggttggcttattatggaattgccaagaatctcatcacgtacttcacaaatgaattgcatgagactaatgtttctgctgctagacacgtcatgacatggcaaggaacatgttacatcactcctcttattggagctttaatagctgatgcttactggggaagatattggactattgcttgtttctctgccatttatttcaccggaatggttgcattgacactctcagcttcagttccgggtcttaagccagcggaatgcattggctctctatgtccaccagcaacaatggttcagtctacggttttattttcagggctttaccttatcgctcttggcactggaggaatcaaaccatgtgtctcatcctttggtgctgatcagtttgataagaccgatccaagcgaacgagtcagaaaagcttctttctttaactggttttacttcactatcaacattggtgcttttgtttcatctactgttctagtttggattcaagagaattatggatgggaattaggattcttgatacctaccgtgttcatgggacttgctactatgagtttcttctttggcacgccgctttatagatttcagaaaccgagaggtagcccgattactagcgtctgccaagttcttgtagccgcataccgtaaatcgaatctcaaggtccctgaagactccacggacgaaggagatgcaaacactaacccgtggaagctatgtaccgtgactcaagtcgaagaagttaagattctgttacgtttggtccccatttgggcctcaggaatcatcttctcagttctccattcacagatttacactctctttgttcaacaaggacggtgcatgaaacgaaccatcggcttattcgaaatccctcccgcaactctcgggatgttcgacactgcaagtgttctcatatctgtcccaatctatgaccgcgtcatcgttcccttagtgagacggttcacaggcttagctaaaggattcaccgagctacaaagaatggggattggtctttttgtctctgttttgagcttgacatttgcagctatcgttgagacggttcggttacagttagctagagatcttgatctagtggaaagtggagacattgttccattaaacatcttttggcaaatccctcagtactttttaatgggcactgctggagttttcttctttgttgggaggattgagtttttctatgagcaatctccagattcaatgagaagcttgtgtagtgcttgggctcttctcactactacactaggaaactacttgagctcgttgatcattacccttgtggcgtatttgagcggaaaagattgttggattccttcagacaacattaacaatggacatcttgattacttcttctggcttttggtcagtcttggatctgttaacatacctgtttttgtcttcttctctgtgaaatatactcatatgaaggtttga

SEQ ID NO: 10 - DNA, 1713 bp, Arabidopsis thaliana
atggaagatgacaaggatatatacacaaaagatggaactcttgacattcacaagaaaccagccaacaagaataaaactggaacctggaaagcttgcagattcattcttggaactgagtgctgtgaaagattagcttactatggaatgagtactaatctcatcaactatctcgagaaacaaatgaatatggaaaacgtctctgcttctaagagtgtcagtaactggtctggaacatgttacgctactcctttgatcggtgcttttatcgccgatgcttatctcggtcgatactggaccatcgcttcctttgtcgtcatctacattgccggaatgacgctattgacgatatcagcttcggttcctggtctaacaccaacctgcagcggagaaacctgtcacgcaacagcgggtcaaaccgctattacattcatagcgctttacttgatcgcactcggaactggagggatcaagccttgtgtctcttcctttggtgctgatcagtttgatgatacagacgaaaaagagaaagagtctaagagctctttctttaactggttctactttgtgatcaacgttggtgcaatgattgcttcctctgttctcgtttggattcagatgaatgttggttggggttggggtttaggtgttcccaccgtcgcaatggctatagccgtcgtgttcttcttcgccggaagcaacttctacaggctgcagaaaccaggaggaagtcctctcacaagaatgctgcaagtcattgtggcttcatgcagaaaatctaaagtgaaaattcctgaagatgaatctcttctctacgagaaccaagacgccgaaagcagtatcataggaagccgcaagctcgaacacaccaaaatattaacgttctttgataaggcagcagtggaaacagagagtgacaacaaaggagcagctaagtcgtcttcatggaagctatgcacagtgacacaagtagaagagctcaaagcactgatccgtctcttaccgatttgggccacagggattgttttcgcttcggtttatagccaaatggggactgtgtttgtactacaaggcaacacactggaccaacacatgggacctaacttcaaaatcccttccgcatcactctccttattcgatacgcttagtgtcctgttttgggcacctgtctacgacaagctaattgttcccttcgcccggaaatacacaggtcacgaacgcggattcacacagcttcaacggattggaatcgggcttgtaatctccatcttttctatggtctctgcgggaatcctcgaggtcgcaaggttaaactacgttcaaacacacaatctttacaatgaagagactatcccgatgacgattttctggcaagttccgcagtattttttggtgggttgcgccgaggttttcacgtttataggtcagcttgagttcttctatgaccaagctcctgatgctatgaggagtctctgctcggctttgtcgctcaccgcaattgcatttgggaactatctgagcacatttctggtgacattggtcactaaagtcacgagatcaggtggaagaccaggctggatcgctaagaacctcaacaatggtcatcttgattacttcttttggctattagctggtctgagtttcttgaatttcttggtctacctttggattgctaaatggtacacttacaagaaaacgaccgggcatgcgctttga

SEQ ID NO: 11 - protein, 622 aa, Arabidopsis thaliana
MITAADFYHVMTAMVPLYVAMILAYGSVKWWKIFTPDQCSGINRFVALFAVPLLSFHFIAANNPYAMNLRFLAADSLQKVIVLSLLFLWCKLSRNGSLDWTITLFSLSTLPNTLVMGIPLLKGMYGNFSGDLMVQIVVLQCIIWYTLMLFLFEYRGAKLLISEQFPDTAGSIVSIHVDSDIMSLDGRQPLETEAEIKEDGKLHVTVRRSNASRSDIYSRRSQGLSATPRPSNLTNAEIYSLQSSRNPTPRGSSFNHTDFYSMMASGGGRNSNFGPGEAVFGSKGPTPRPSNYEEDGGPAKPTAAGTAAGAGRFHYQSGGSGGGGGAHYPAPNPGMFSPNTGGGGGTAAKGNAPVVGGKRQDGNGRDLHMFVWSSSASPVSDVFGGGGGNHHADYSTATNDHQKDVKISVPQGNSNDNQYVEREEFSFGNKDDDSKVLATDGGNNISNKTTQAKVMPPTSVMTRLILIMVWRKLIRNPNSYSSLFGITWSLISFKWNIEMPALIAKSISILSDAGLGMAMFSLGLFMALNPRIIACGNRRAAFAAAMRFVVGPAVMLVASYAVGLRGVLLHVAIIQAALPQGIVPFVFAKEYNVHPDILSTAVIFGMLIALPITLLYYILLGL

SEQ ID NO: 12 - DNA, 2270 bp, Arabidopsis thaliana
aacactcactttactcttttttccctcttcaccacttctctctcaaactaaagacaaaagctcttctctcttccctctctcttctccggcgaacaaaagatgattacggcggcggacttctaccacgttatgacggctatggttccgttatacgtagctatgatcctcgcttacggctctgtcaaatggtggaaaatcttcacaccagaccaatgctccggcataaaccgtttcgtcgctctcttcgccgttcctctcctctctttccacttcatcgccgctaacaacccttacgccatgaacctccgtttcctcgccgcagattctctccagaaagtcattgtcctctctctcctcttcctctggtgcaaactcagccgcaacggttctttagattggaccataactctcttctctctctcgacactccccaacactctagtcatggggatacctcttctcaaaggcatgtatggtaatttctccggcgacctcatggttcaaatcgttgttcttcagtgtatcatttggtacacactcatgctctttctctttgagtaccgtggagctaagcttttgatctccgagcagtttccagacacagcaggatctattgtttcgattcatgttgattccgacattatgtctttagatggaagacaacctttggaaactgaagctgagattaaagaagatgggaagcttcatgttactgttcgtcgttctaatgcttcaaggtctgatatttactcgagaaggtctcaaggcttatctgcgacacctagaccttcgaatctaaccaacgctgagatatattcgcttcagagttcaagaaacccaacgccacgtggctctagttttaatcatactgatttttactcgatgatggcttctggtggtggtcggaactctaactttggtcctggagaagctgtgtttggttctaaaggtcctactccgagaccttccaactacgaagaagacggtggtcctgctaaaccgacggctgctggaactgctgctggagctgggaggtttcattatcaatctggaggaagtggtggcggtggaggagcgcattatccggcgccgaacccagggatgttttcgcccaacactggcggtggtggaggcacggcggcgaaaggaaacgctccggtggttggtgggaaaagacaagacggaaacggaagagatcttcacatgtttgtgtggagctcaagtgcttcgccggtctcagatgtgttcggcggtggaggaggaaaccaccacgccgattactccaccgctacgaacgatcatcaaaaggacgttaagatctctgtacctcaggggaatagtaacgacaaccagtacgtggagagggaagagtttagtttcggtaacaaagacgatgatagcaaagtattggcaacggacggtgggaacaacataagcaacaaaacgacgcaggctaaggtgatgccaccaacaagtgtgatgacaagactcattctcattatggtttggaggaaacttattcgtaatcccaactcttactccagtttattcggcatcacctggtccctcatttccttcaagtggaacattgaaatgccagctcttatagcaaagtctatctccatactctcagatgcaggtctaggcatggctatgttcagtcttgggttgttcatggcgttaaacccaagaataatagcttgtggaaacagaagagcagcttttgcggcggctatgagatttgtcgttggacctgccgtcatgctcgttgcttcttatgccgttggcctccgtggcgtcctcctccatgttgccattatccaggcagctttgccgcaaggaatagtaccgtttgtgtttgccaaagagtataatgtgcatcctgacattcttagcactgcggtgatatttgggatgttgatcgcgttgcccataactcttctctactacattctcttgggtctatgaagagatattaccaaaacacagggactttgttttattcttttgtgggatgatgaattgtgaaaagaacaatgccctttttgttgaaaacccacaaattaaatcagaagcagctttagagaatctttgaggataattgaagctcttgaagaagagaagaagaaggagacttaagtaggagctcagcaagttttacctttttcttaattttaatgaacattcgtgtttcctcttttggtaggttttaggaatttgtaaaagctttggctacttttagtgaattaaaaacgttaaggaaaatatca

SEQ ID NO: 13 - DNA, 1869 bp, Arabidopsis thaliana
atgattacggcggcggacttctaccacgttatgacggctatggttccgttatacgtagctatgatcctcgcttacggctctgtcaaatggtggaaaatcttcacaccagaccaatgctccggcataaaccgtttcgtcgctctcttcgccgttcctctcctctctttccacttcatcgccgctaacaacccttacgccatgaacctccgtttcctcgccgcagattctctccagaaagtcattgtcctctctctcctcttcctctggtgcaaactcagccgcaacggttctttagattggaccataactctcttctctctctcgacactccccaacactctagtcatggggatacctcttctcaaaggcatgtatggtaatttctccggcgacctcatggttcaaatcgttgttcttcagtgtatcatttggtacacactcatgctctttctctttgagtaccgtggagctaagcttttgatctccgagcagtttccagacacagcaggatctattgtttcgattcatgttgattccgacattatgtctttagatggaagacaacctttggaaactgaagctgagattaaagaagatgggaagcttcatgttactgttcgtcgttctaatgcttcaaggtctgatatttactcgagaaggtctcaaggcttatctgcgacacctagaccttcgaatctaaccaacgctgagatatattcgcttcagagttcaagaaacccaacgccacgtggctctagttttaatcatactgatttttactcgatgatggcttctggtggtggtcggaactctaactttggtcctggagaagctgtgtttggttctaaaggtcctactccgagaccttccaactacgaagaagacggtggtcctgctaaaccgacggctgctggaactgctgctggagctgggaggtttcattatcaatctggaggaagtggtggcggtggaggagcgcattatccggcgccgaacccagggatgttttcgcccaacactggcggtggtggaggcacggcggcgaaaggaaacgctccggtggttggtgggaaaagacaagacggaaacggaagagatcttcacatgtttgtgtggagctcaagtgcttcgccggtctcagatgtgttcggcggtggaggaggaaaccaccacgccgattactccaccgctacgaacgatcatcaaaaggacgttaagatctctgtacctcaggggaatagtaacgacaaccagtacgtggagagggaagagtttagtttcggtaacaaagacgatgatagcaaagtattggcaacggacggtgggaacaacataagcaacaaaacgacgcaggctaaggtgatgccaccaacaagtgtgatgacaagactcattctcattatggtttggaggaaacttattcgtaatcccaactcttactccagtttattcggcatcacctggtccctcatttccttcaagtggaacattgaaatgccagctcttatagcaaagtctatctccatactctcagatgcaggtctaggcatggctatgttcagtcttgggttgttcatggcgttaaacccaagaataatagcttgtggaaacagaagagcagcttttgcggcggctatgagatttgtcgttggacctgccgtcatgctcgttgcttcttatgccgttggcctccgtggcgtcctcctccatgttgccattatccaggcagctttgccgcaaggaatagtaccgtttgtgtttgccaaagagtataatgtgcatcctgacattcttagcactgcggtgatatttgggatgttgatcgcgttgcccataactcttctctactacattctcttgggtctatga

SEQ ID NO: 14 - protein, 647 aa, Arabidopsis thaliana
MITGKDMYDVLAAMVPLYVAMILAYGSVRWWGIFTPDQCSGINRFVAVFAVPLLSFHFISSNDPYAMNYHFLAADSLQKVVILAALFLWQAFSRRGSLEWMITLFSLSTLPNTLVMGIPLLRAMYGDFSGNLMVQIVVLQSIIWYTLMLFLFEFRGAKLLISEQFPETAGSITSFRVDSDVISLNGREPLQTDAEIGDDGKLHVVVRRSSAASSMISSFNKSHGGGLNSSMITPRASNLTGVEIYSVQSSREPTPRASSFNQTDFYAMFNASKAPSPRHGYTNSYGGAGAGPGGDVYSLQSSKGVTPRTSNFDEEVMKTAKKAGRGGRSMSGELYNNNSVPSYPPPNPMFTGSTSGASGVKKKESGGGGSGGGVGVGGQNKEMNMFVWSSSASPVSEANAKNAMTRGSSTDVSTDPKVSIPPHDNLATKAMQNLIENMSPGRKGHVEMDQDGNNGGKSPYMGKKGSDVEDGGPGPRKQQMPPASVMTRLILIMVWRKLIRNPNTYSSLFGLAWSLVSFKWNIKMPTIMSGSISILSDAGLGMAMFSLGLFMALQPKIIACGKSVAGFAMAVRFLTGPAVIAATSIAIGIRGDLLHIAIVQAALPQGIVPFVFAKEYNVHPDILSTAVIFGMLVALPVTVLYYVLLGL

SEQ ID NO: 15 - DNA, 2295 bp, Arabidopsis thaliana
cacaccacatatactcatctatatctctatttttcttcttcttctctctctcgccggaaaaagtaaatcaaaatgatcaccggcaaagacatgtacgatgttttagcggctatggtgccgctatacgttgctatgatattagcctatggttcggtacggtggtgggggatattcacaccggaccaatgttccggtataaaccggttcgttgcggttttcgcggttcctcttctctctttccatttcatctcctccaatgatccttatgcaatgaattaccacttcctcgctgctgattctcttcagaaagtcgttatcctcgccgcactctttctttggcaggcgtttagccgcagaggaagcctagaatggatgataacgctcttttcactatcaacactgcctaacacgttggtaatgggaatcccattgcttagggcgatgtacggagacttctccggtaacctaatggtgcagatcgtggtgcttcagagcatcatatggtatacattaatgctcttcttgtttgagttccgtggggctaagcttctcatctccgagcagttcccggagacggctggttcaattacttccttcagagttgactctgatgttatctctcttaatggccgtgaacccctccagaccgatgcggagataggagacgacggaaagctacacgtggtggttcgaagatcaagtgccgcctcatcaatgatctcttcattcaacaaatctcacggcggaggacttaactcctccatgataacgccgcgagcttcaaatctcaccggcgtagagatttactccgttcaatcgtcacgagagccgacgccgagagcttctagctttaatcagacagatttctacgcaatgtttaacgcaagcaaagctccaagccctcgtcacggttacactaatagctacggcggcgctggagctggtccaggtggagatgtttactcacttcagtcttctaaaggcgtgacgccgagaacgtcaaattttgatgaggaagttatgaagacggcgaagaaagcaggaagaggaggcagaagtatgagtggggaattatacaacaataatagtgttccgtcgtacccaccgccgaacccaatgttcacggggtcaacgagtggagcaagtggagtcaagaaaaaggaaagtggtggcggaggaagcggtggcggagtaggagtaggaggacaaaacaaggagatgaacatgttcgtgtggagttcgagtgcttctccggtgtcggaagccaacgcgaagaatgctatgaccagaggttcttccaccgatgtatccaccgaccctaaagtttctattcctcctcacgacaacctcgctactaaagcgatgcagaatctgatagagaacatgtcaccgggaagaaaagggcatgtggaaatggaccaagacggtaataacgggggaaagtcaccttacatgggcaaaaaaggtagcgacgtggaagacggcggtcccggtcctaggaaacagcagatgccgccggcgagtgtgatgacgagactaattctgataatggtttggagaaaactcattcgaaaccctaacacttactctagtctctttggccttgcttggtcccttgtctctttcaagtggaatataaagatgccaacgataatgagtggatcgatttcgatattatctgatgctggtcttggaatggctatgtttagtcttggtctatttatggcattgcaaccaaagattattgcgtgcggaaaatcagtagcagggtttgcgatggccgtaaggttcttgactggaccagccgtgatcgcagccacctcaatagcaattggtattcgaggtgatctcctccatatcgccatcgttcaggctgctcttcctcaaggaatcgttccttttgttttcgccaaagaatataacgtccatcctgatattctcagcactgcggttatattcggaatgctggttgctttgcctgtaacagtactctactacgttcttttggggctttaagttattatcaaaacgtatttgcaaataaaaggcgatacgacccaaaggtgattttttttcaaacgaaaaagaataattacaagaacgaaaaaagactaattccaggtcaggcttaggtgtatgggaccatgcaatgtcgcattaattaaattatagcatatgatagtcgaaaatttagataactttgtataattaattatatgcacatgcatgtacgtgactttgtagtttttgttacatttattaaatttttgggatgtgcaagtacaattatttact

SEQ ID NO: 16 - DNA, 1944 bp, Arabidopsis thaliana
atgatcaccggcaaagacatgtacgatgttttagcggctatggtgccgctatacgttgctatgatattagcctatggttcggtacggtggtgggggatattcacaccggaccaatgttccggtataaaccggttcgttgcggttttcgcggttcctcttctctctttccatttcatctcctccaatgatccttatgcaatgaattaccacttcctcgctgctgattctcttcagaaagtcgttatcctcgccgcactctttctttggcaggcgtttagccgcagaggaagcctagaatggatgataacgctcttttcactatcaacactgcctaacacgttggtaatgggaatcccattgcttagggcgatgtacggagacttctccggtaacctaatggtgcagatcgtggtgcttcagagcatcatatggtatacattaatgctcttcttgtttgagttccgtggggctaagcttctcatctccgagcagttcccggagacggctggttcaattacttccttcagagttgactctgatgttatctctcttaatggccgtgaacccctccagaccgatgcggagataggagacgacggaaagctacacgtggtggttcgaagatcaagtgccgcctcatcaatgatctcttcattcaacaaatctcacggcggaggacttaactcctccatgataacgccgcgagcttcaaatctcaccggcgtagagatttactccgttcaatcgtcacgagagccgacgccgagagcttctagctttaatcagacagatttctacgcaatgtttaacgcaagcaaagctccaagccctcgtcacggttacactaatagctacggcggcgctggagctggtccaggtggagatgtttactcacttcagtcttctaaaggcgtgacgccgagaacgtcaaattttgatgaggaagttatgaagacggcgaagaaagcaggaagaggaggcagaagtatgagtggggaattatacaacaataatagtgttccgtcgtacccaccgccgaacccaatgttcacggggtcaacgagtggagcaagtggagtcaagaaaaaggaaagtggtggcggaggaagcggtggcggagtaggagtaggaggacaaaacaaggagatgaacatgttcgtgtggagttcgagtgcttctccggtgtcggaagccaacgcgaagaatgctatgaccagaggttcttccaccgatgtatccaccgaccctaaagtttctattcctcctcacgacaacctcgctactaaagcgatgcagaatctgatagagaacatgtcaccgggaagaaaagggcatgtggaaatggaccaagacggtaataacgggggaaagtcaccttacatgggcaaaaaaggtagcgacgtggaagacggcggtcccggtcctaggaaacagcagatgccgccggcgagtgtgatgacgagactaattctgataatggtttggagaaaactcattcgaaaccctaacacttactctagtctctttggccttgcttggtcccttgtctctttcaagtggaatataaagatgccaacgataatgagtggatcgatttcgatattatctgatgctggtcttggaatggctatgtttagtcttggtctatttatggcattgcaaccaaagattattgcgtgcggaaaatcagtagcagggtttgcgatggccgtaaggttcttgactggaccagccgtgatcgcagccacctcaatagcaattggtattcgaggtgatctcctccatatcgccatcgttcaggctgctcttcctcaaggaatcgttccttttgttttcgccaaagaatataacgtccatcctgatattctcagcactgcggttatattcggaatgctggttgctttgcctgtaacagtactctactacgttcttttggggctttaa

SEQ ID NO: 17 - DNA, 9574 bp, Artificial Sequence
  Synthetic construct
ccccagcctcgactagatgcggggttctcatcatcatcatcatcatggtatggctagcatgactggtggacagcaaatgggtcgggatctgtacgacgatgacgataaggatccgggcctcgaggttggtaccgatatcacaagtttgtacaaaaaagctgaaatgtctcttcctgaaactaaatctgatgatatccttcttgatgcttgggacttccaaggccgtcccgccgatcgctcaaaaaccggcggctgggccagcgccgccatgattctttgtattgaggccgtggagaggctgacgacgttaggtatcggagttaatctggtgacgtatttgacgggaactatgcatttaggcaatgcaactgcggctaacaccgttaccaatttcctcggaacttctttcatgctctgtctcctcggtggcttcatcgccgatacctttctcggcaggtacctaacgattgctatattcgccgcaatccaagccacgggtgtttcaatcttaactctatcaacaatcataccgggacttcgaccaccaagatgcaatccaacaacgtcgtctcactgcgaacaagcaagtggaatacaactgacggtcctatacttagccttatacctcaccgctctaggaacgggaggcgtgaaggctagtgtctcgggtttcgggtcggaccaattcgatgagaccgaaccaaaagaacgatcgaaaatgacatatttcttcaaccgtttcttcttttgtatcaacgttggctctcttttagctgtgacggtccttgtctacgtacaagacgatgttggacgcaaatggggctatggaatttgcgcgtttgcgatcgtgcttgcactcagcgttttcttggccggaacaaaccgctaccgtttcaagaagttgatcggtagcccgatgacgcaggttgctgcggttatcgtggcggcgtggaggaataggaagctcgagctgccggcagatccgtcctatctctacgatgtggatgatattattgcggcggaaggttcgatgaagggtaaacaaaagctgccacacactgaacaattccgttcattagataaggcagcaataagggatcaggaagcgggagttacctcgaatgtattcaacaagtggacactctcaacactaacagatgttgaggaagtgaaacaaatcgtgcgaatgttaccaatttgggcaacatgcatcctcttctggaccgtccacgctcaattaacgacattatcagtcgcacaatccgagacattggaccgttccatcgggagcttcgagatccctccagcatcgatggcagtcttctacgtcggtggcctcctcctaaccaccgccgtctatgaccgcgtcgccattcgtctatgcaaaaagctattcaactacccccatggtctaagaccgcttcaacggatcggtttggggcttttcttcggatcaatggctatggctgtggctgctttggtcgagctcaaacgtcttagaactgcacacgctcatggtccaacagtcaaaacgcttcctctagggttttatctactcatcccacaatatcttattgtcggtatcggcgaagcgttaatctacacaggacagttagatttcttcttgagagagtgccctaaaggtatgaaagggatgagcacgggtctattgttgagcacattggcattaggctttttcttcagctcggttctcgtgacaatcgtcgagaaattcaccgggaaagctcatccatggattgccgatgatctcaacaagggccgtctttacaatttctactggcttgtggccgtacttgttgccttgaacttcctcattttcctagttttctccaagtggtacgtttacaaggaaaaaagactagctgaggtggggattgagttggatgatgagccgagtattccaatgggtcatgctttcttgtacaaagtggtgatatcgactagtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggtaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaacaaaaattgataagtgaggaagatttataagctcgaggggcccgatccggctgctaacaaagcccgaaagggtcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaraattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatata

SEQ ID NO: 18 - DNA, 9713 bp, Artificial Sequence
  Synthetic construct
gtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctcgatctagaaataattttgtttaactttaagaaggagatatacatatgcggggttctcatcatcatcatcatcatggtatggctagcatgactggtggacagcaaatgggtcgggatctgtacgacgatgacgataaggatccgggcctcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggtaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggtaccgatatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatgggcttccatgtcggcagaatgcttaatgaattacacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaacttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatatcgactagtgtttctaaaggtgaagaattgtttacgggcgtcgtcccgatcctcgtggaactcgacggggatgttaacgggcataagttttcggtcagcggggaaggggagggggacgcgacgtatgggaagctcactctcaagctgatctgtacgacggggaaactcccggtcccgtggccgacgctggtcacgacgctgggatacgggctccaatgctttgcgaggtatccggaccacatgaaacagcatgactttttcaaatcggcgatgccggagggatacgtgcaggaacggacgatctttttcaaagacgatgggaactataagacgcgggcggaagtcaagtttgaaggggacacgctcgtcaaccggatcgaactcaaggggattgacttcaaagaggatgggaacatactcggccataagctcgaatacaattacaactcgcataacgtatacatcaccgcggataagcaaaagaacgggatcaaagccaatttcaaaatccggcataacatagaggatgggggggtccaactggcggatcactatcagcaaaacacgccgataggggatgggccggtcctcctcccggataaccattacctctcgtaccaaagcgcgctctcgaaggacccgaatgagaaacgggaccacatggttctcctggagttcgtcacggcggcgggcatagaacaaaaattgataagtgaggaagatttataagggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctata

SEQ ID NO: 19 - DNA, 10267 bp, Artificial Sequence
  Synthetic construct
ccccagcctcgactagatgcggggttctcatcatcatcatcatcatggtatggctagcatgactggtggacagcaaatgggtcgggatctgtacgacgatgacgataaggatccgggcctcgagatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacgctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacgtacgtcgagcagcacgaggtggctgtggccagatactgcgacctccctagcaaactggggcacagaggtaccgatatcacaagtttgtacaaaaaagctgaaatgtctcttcctgaaactaaatctgatgatatccttcttgatgcttgggacttccaaggccgtcccgccgatcgctcaaaaaccggcggctgggccagcgccgccatgattctttgtattgaggccgtggagaggctgacgacgttaggtatcggagttaatctggtgacgtatttgacgggaactatgcatttaggcaatgcaactgcggctaacaccgttaccaatttcctcggaacttctttcatgctctgtctcctcggtggcttcatcgccgatacctttctcggcaggtacctaacgattgctatattcgccgcaatccaagccacgggtgtttcaatcttaactctatcaacaatcataccgggacttcgaccaccaagatgcaatccaacaacgtcgtctcactgcgaacaagcaagtggaatacaactgacggtcctatacttagccttatacctcaccgctctaggaacgggaggcgtgaaggctagtgtctcgggtttcgggtcggaccaattcgatgagacggaaccaaaagaacgatcgaaaatgacatatttcttcaaccgtttcttcttttgtatcaacgttggctctcttttagctgtgacggtccttgtctacgtacaagacgatgttggacgcaaatggggctatggaatttgcgcgtttgcgatcgtgcttgcactcagcgttttcttggccggaacaaaccgctaccgtttcaagaagttgatcggtagcccgatgacgcaggttgctgcggttatcgtggcggcgtggaggaataggaagctcgagctgccggcagatccgtcctatctctacgatgtggatgatattattgcggcggaaggttcgatgaagggtaaacaaaagctgccacacactgaacaattccgttcattagataaggcagcaataagggatcaggaagcgggagttacctcgaatgtattcaacaagtggacactctcaacactaacagatgttgaggaagtgaaacaaatcgtgcgaatgttaccaatttgggcaacatgcatcctcttctggaccgtccacgctcaattaacgacattatcagtcgcacaatccgagacattggaccgttccatcgggagcttcgagatccctccagcatcgatggcagtcttctacgtcggtggcctcctcctaaccaccgccgtctatgaccgcgtcgccattcgtctatgcaaaaagctattcaactacccccatggtctaagaccgcttcaacggatcggtttggggcttttcttcggatcaatggctatggctgtggctgctttggtcgagctcaaacgtcttagaactgcacacgctcatggtccaacagtcaaaacgcttcctctagggttttatctactcatcccacaatatcttattgtcggtatcggcgaagcgttaatctacacaggacagttagatttcttcttgagagagtgccctaaaggtatgaaagggatgagcacgggtctattgttgagcacattggcattaggctttttcttcagctcggttctcgtgacaatcgtcgagaaattcaccgggaaagctcatccatggattgccgatgatctcaacaagggccgtctttacaatttctactggcttgtggccgtacttgttgccttgaacttcctcattttcctagttttctccaagtggtacgtttacaaggaaaaaagactagctgaggtggggattgagttggatgatgagccgagtattccaatgggtcatgctttcttgtacaaagtggtgatatcgactagtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggtaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaacaaaaattgataagtgaggaagatttataagctcgaggggcccgatccggctgctaacaaagcccgaaagggtcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaraattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatata

SEQ ID NO: 20 - DNA, 10327 bp, Artificial Sequence
  Synthetic construct
ccccagcctcgactagatgcggggttctcatcatcatcatcatcatggtatggctagcatgactggtggacagcaaatgggtcgggatctgtacgacgatgacgataaggatccgggcctcgaggtttctaaaggtgaagaattgtttacgggcgtcgtcccgatcctcgtggaactcgacggggatgttaacgggcataagttttcggtcagcggggaaggggagggggacgcgacgtatgggaagctcactctcaagctgatctgtacgacggggaaactcccggtcccgtggccgacgctggtcacgacgctgggatacgggctccaatgctttgcgaggtatccggaccacatgaaacagcatgactttttcaaatcggcgatgccggagggatacgtgcaggaacggacgatctttttcaaagacgatgggaactataagacgcgggcggaagtcaagtttgaaggggacacgctcgtcaaccggatcgaactcaaggggattgacttcaaagaggatgggaacatactcggccataagctcgaatacaattacaactcgcataacgtatacatcaccgcggataagcaaaagaacgggatcaaagccaatttcaaaatccggcataacatagaggatgggggggtccaactggcggatcactatcagcaaaacacgccgataggggatgggccggtcctcctcccggataaccattacctctcgtaccaaagcgcgctcttcaaggacccgaatgagaaacgggaccacatggttctcctggagttcctcacggcggcgggcatatctagagatatcacaagtttgtacaaaaaagctgaactgcagatgattacggcggcggacttctaccacgttatgacggctatggttccgttatacgtagctatgatcctcgcttacggctctgtcaaatggtggaaaatcttcacaccagaccaatgctccggcataaaccgtttcgtcgctctcttcgccgttcctctcctctctttccacttcatcgccgctaacaacccttacgccatgaacctccgtttcctcgccgcagattctctccagaaagtcattgtcctctctctcctcttcctctggtgcaaactcagccgcaacggttctttagattggaccataactctcttctctctctcgacactccccaacactctagtcatggggatacctcttctcaaaggcatgtatggtaatttctccggcgacctcatggttcaaatcgttgttcttcagtgtatcatttggtacacactcatgctctttctctttgagtaccgtggagctaagcttttgatctccgagcagtttccagacacagcaggatctattgtttcgattcatgttgattccgacattatgtctttagatggaagacaacctttggaaactgaagctgagattaaagaagatgggaagcttcatgttactgttcgtcgttctaatgcttcaaggtctgatatttactcgagaaggtctcaaggcttatctgcgacacctagaccttcgaatctaaccaacgctgagatatattcgcttcagagttcaagaaacccaacgccacgtggctctagttttaatcatactgatttttactcgatgatggcttctggtggtggtcggaactctaactttggtcctggagaagctgtgtttggttctaaaggtcctactccgagaccttccaactacgaagaagacggtggtcctgctaaaccgacggctgctggaactgctgctggagctgggaggtttcattatcaatctggaggaagtggtggcggtggaggagcgcattatccggcgccgaacccagggatgttttcgcccaacactggcggtggtggaggcacggcggcgaaaggaaacgctccggtggttggtgggaaaagacaagacggaaacggaagagatcttcacatgtttgtgtggagctcaagtgcttcgccggtctcagatgtgttcggcggtggaggaggaaaccaccacgccgattactccaccgctacgaacgatcatcaaaaggacgttaagatctctgtacctcaggggaatagtaacgacaaccagtacgtggagagggaagagtttagtttcggtaacaaagacgatgatagcaaagtattggcaacggacggtgggaacaacataagcaacaaaacgacgcaggctaaggtgatgccaccaacaagtgtgatgacaagactcattctcattatggtttggaggaaacttattcgtaatcccaactcttactccagtttattcggcatcacctggtccctcatttccttcaagtggaacattgaaatgccagctcttatagcaaagtctatctccatactctcagatgcaggtctaggcatggctatgttcagtcttgggttgttcatggcgttaaacccaagaataatagcttgtggaaacagaagagcagcttttgcggcggctatgagatttgtcgttggacctgccgtcatgctcgttgcttcttatgccgttggcctccgtggcgtcctcctccatgttgccattatccaggcagctttgccgcaaggaatagtaccgtttgtgtttgccaaagagtataatgtgcatcctgacattcttagcactgcggtgatatttgggatgttgatcgcgttgcccataactcttctctactacattctcttgggtctaacgcgtgctttcttgtacaaagtggtgatatcgactagtgaattcctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgttcaaagaccccaacgagaagcgcgatcacatggtcctgctggagttcctgaccgccgccgggatcgaacaaaaattgataagtgaggaagatttataagctcgaggggcccgatccggctgctaacaaagcccgaaagggtcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaraattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatata

SEQ ID NO: 21 - DNA, 3666 bp, Artificial Sequence
  Synthetic construct
atgatggtttctaaaggtgaagaattgtttacgggcgtcgtcccgatcctcgtggaactcgacggggatgttaacgggcataagttttcggtcagcggggaaggggagggggacgcgacgtatgggaagctcactctcaagctgatctgtacgacggggaaactcccggtcccgtggccgacgctggtcacgacgctgggatacgggctccaatgctttgcgaggtatccggaccacatgaaacagcatgactttttcaaatcggcgatgccggagggatacgtgcaggaacggacgatctttttcaaagacgatgggaactataagacgcgggcggaagtcaagtttgaaggggacacgctcgtcaaccggatcgaactcaaggggattgacttcaaagaggatgggaacatactcggccataagctcgaatacaattacaactcgcataacgtatacatcaccgcggataagcaaaagaacgggatcaaagccaatttcaaaatccggcataacatagaggatgggggggtccaactggcggatcactatcagcaaaacacgccgataggggatgggccggtcctcctcccggataaccattacctctcgtaccaaagcgcgctctcgaaggacccgaatgagaaacgggaccacatggttctcctggagttcgtcacggcggcgggcataggtaccgatatcacaagtttgtacaaaaaagcaggctccgcggccgcccccttcaccatggcagaacaaaagagtagtaacggaggaggaggaggaggagatgttgttatcaatgttccagttgaggaagcatcaaggcgttccaaggaaatggcttcaccagagtctgagaaaggagttccctttagtaaaagcccttctcctgaaatctctaagcttgttggtagtcctaacaagcctcctagagctccaaatcagaacaatgtgggtctaactcagaggaaatcttttgcaaggtcggtttactcaaaacccaagtcccggtttgttgatccatcttgtcctgtagacacaagtattctagaggaggaagttagggagcaacttggtgctggtttttcttttagtagagcttctccgaataacaaatctaataggagtgtcgggtcaccagcaccggttactccaagtaaagtcgttgttgagaaagatgaggatgaggaaatctacaagaaggttaagctgaacagagagatgcgcagtaagataagtacattggctttgatagagtcagctttctttgtggtgattttgagcgctttggttgcgagtttaaccattaatgtcctgaaacatcacaccttctgggggctagaagtctggaaatggtgtgtgcttgtgatggttatattcagtggaatgttggtgacaaactggttcatgcgtttgattgtgttcctcatagaaacaaactttcttttgaggagaaaagtgctctactttgtgcacggcttgaagaagagcgtccaagttttcatttggctctgcttgattcttgttgcttggatattgttgttcaaccacgacgtgaaacggtcccccgcagccaccaaagtcctcaaatgtattaccaggactcttatttccattcttacaggggcattcttttggctggtgaaaacactcttgttgaaaatccttgcagcgaatttcaacgtcaataactttttcgataggattcaagattctgttttccaccagtatgttctacaaacgctctcgggtcttccacttatggaagaggcagagagggtcgggcgtgagccaagcacaggccatttgagtttcgcgactgtagtgaaaaaaggaacggttaaagagaagaaagtgattgatatggggaaagttcataagatgaagcgggagaaagtttcggcttggactatgcgagttttgatggaagcggttagaacttcaggtctctctactatctctgacacattggacgaaacagcatacggcgaggggaaagagcaagctgacagagaaattactagtgagatggaggctttggctgctgcttaccatgtcttcagaaatgttgctcagcccttcttcaattacatagaggaagaggacttgcttaggtttatgattaaggaagaggttgatcttgtgttcccattgtttgatggtgccgctgagaccgggagaattacaagaaaagctttcacagaatgggtggttaaggtgtacacgagccggagagctttagcgcattccttaaacgacacaaaaacagcggttaagcagttaaacaaacttgtgacagcaatcttgatggtggttaccgttgtcatttggctgctccttctagaagtagcaacgactaaggttttgctgttcttctccacccaactcgtggctctggcttttataatcggaagcacatgcaaaaacctctttgaatccattgtgttcgtattcgtcatgcatccttatgatgtcggtgatcgatgtgttgttgacggtgtcgcgatgctggtggaagaaatgaatctcttaacgacagtgttcttgaagcttaacaacgagaaagtgtattatccgaacgctgttttggccacgaaaccgataagcaattacttcagaagtccgaatatgggagaaacagtggaattctctatctctttctcgacaccagtctctaagatagcacatctcaaagaaagaatcgccgagtacttggagcagaacccgcaacattgggcaccggttcactcggtggtggtgaaggagatagagaacatgaacaagctgaagatggccctatacagtgaccacaccatcacgtttcaggaaaacagagagaggaatcttagaagaaccgaactttctttggccattaagagaatgttggaggaccttcacatcgactacactctccttcctcaagacattaatctcacaaagaagaacaagggtgggcgcgccgacccagctttcttgtacaaagtggtgatatcgactagtaccacaatgggcgtaatcaagcccgacatgaagatcaagctgaagatggagggcaacgtgaatggccacgccttcgtgatcgagggcgagggcgagggcaagccctacgacggcaccaacaccatcaacctggaggtgaaggagggagcccccctgcccttctcctacgacattctgaccaccgcgttcgcctacggcaacagggccttcaccaagtaccccgacgacatccccaactacttcaagcagtccttccccgagggctactcttgggagcgcaccatgaccttcgaggacaagggcatcgtgaaggtgaagtccgacatctccatggaggaggactccttcatctacgagatacacctcaagggcgagaacttcccccccaacggccccgtgatgcagaagaagaccaccggctgggacgcctccaccgagaggatgtacgtgcgcgacggcgtgctgaagggcgacgtcaagcacaagctgctgctggagggcggcggccaccaccgcgttgacttcaagaccatctacagggccaagaaggcggtgaagctgcccgactatcactttgtggaccaccgcatcgagatcctgaaccacgacaaggactacaacaaggtgaccgtttacgagagcgccgtggcccgcaactcc

SEQ ID NO: 22 - DNA, 2202 bp, Arabidopsis thaliana
atggcagaacaaaagagtagtaacggaggaggaggaggaggagatgttgttatcaatgttccagttgaggaagcatcaaggcgttccaaggaaatggcttcaccagagtctgagaaaggagttccctttagtaaaagcccttctcctgaaatctctaagcttgttggtagtcctaacaagcctcctagagctccaaatcagaacaatgtgggtctaactcagaggaaatcttttgcaaggtcggtttactcaaaacccaagtcccggtttgttgatccatcttgtcctgtagacacaagtattctagaggaggaagttagggagcaacttggtgctggtttttcttttagtagagcttctccgaataacaaatctaataggagtgtcgggtcaccagcaccggttactccaagtaaagtcgttgttgagaaagatgaggatgaggaaatctacaagaaggttaagctgaacagagagatgcgcagtaagataagtacattggctttgatagagtcagctttctttgtggtgattttgagcgctttggttgcgagtttaaccattaatgtcctgaaacatcacaccttctgggggctagaagtctggaaatggtgtgtgcttgtgatggttatattcagtggaatgttggtgacaaactggttcatgcgtttgattgtgttcctcatagaaacaaactttcttttgaggagaaaagtgctctactttgtgcacggcttgaagaagagcgtccaagttttcatttggctctgcttgattcttgttgcttggatattgttgttcaaccacgacgtgaaacggtcccccgcagccaccaaagtcctcaaatgtattaccaggactcttatttccattcttacaggggcattcttttggctggtgaaaacactcttgttgaaaatccttgcagcgaatttcaacgtcaataactttttcgataggattcaagattctgttttccaccagtatgttctacaaacgctctcgggtcttccacttatggaagaggcagagagggtcgggcgtgagccaagcacaggccatttgagtttcgcgactgtagtgaaaaaaggaacggttaaagagaagaaagtgattgatatggggaaagttcataagatgaagcgggagaaagtttcggcttggactatgcgagttttgatggaagcggttagaacttcaggtctctctactatctctgacacattggacgaaacagcatacggcgaggggaaagagcaagctgacagagaaattactagtgagatggaggctttggctgctgcttaccatgtcttcagaaatgttgctcagcccttcttcaattacatagaggaagaggacttgcttaggtttatgattaaggaagaggttgatcttgtgttcccattgtttgatggtgccgctgagaccgggagaattacaagaaaagctttcacagaatgggtggttaaggtgtacacgagccggagagctttagcgcattccttaaacgacacaaaaacagcggttaagcagttaaacaaacttgtgacagcaatcttgatggtggttaccgttgtcatttggctgctccttctagaagtagcaacgactaaggttttgctgttcttctccacccaactcgtggctctggcttttataatcggaagcacatgcaaaaacctctttgaatccattgtgttcgtattcgtcatgcatccttatgatgtcggtgatcgatgtgttgttgacggtgtcgcgatgctggtggaagaaatgaatctcttaacgacagtgttcttgaagcttaacaacgagaaagtgtattatccgaacgctgttttggccacgaaaccgataagcaattacttcagaagtccgaatatgggagaaacagtggaattctctatctctttctcgacaccagtctctaagatagcacatctcaaagaaagaatcgccgagtacttggagcagaacccgcaacattgggcaccggttcactcggtggtggtgaaggagatagagaacatgaacaagctgaagatggccctatacagtgaccacaccatcacgtttcaggaaaacagagagaggaatcttagaagaaccgaactttctttggccattaagagaatgttggaggaccttcacatcgactacactctccttcctcaagacattaatctcacaaagaagaac

(19) United States
(12) Patent Application Publication
(10) Pub. No.: US 2015/0125893 A1
(43) Pub. Date: May 7, 2015

(54) TRANSPORTER BIOSENSORS

(71) Applicant: Carnegie Institute of Washington, Washington, DC (US)

(72) Inventors: Wolf B. Frommer, Washington, DC (US); Cheng-Hsun Ho, Washington, DC (US)

(21) Appl. No.: 14/535,094

(22) Filed: Nov. 6, 2014

Related U.S. Application Data
(60) Provisional application No. 61/900,584, filed on Nov. 6, 2013.

(51) Int. Cl. G01N 33/58; G01N 33/50; C07K 14/415

(52) U.S. Cl. CPC G01N 33/582; C07K 14/415; G01N 33/5097; C07K 2319/60
USPC 435/29; 530/370; 536/23.4; 435/320.1; 800/298; 435/69.7; 435/252.33; 435/252.31; 435/252.34; 435/252.35; 435/348; 435/367; 435/358; 435/365; 435/369; 435/357; 435/254.2; 435/254.21; 435/254.23

(57) ABSTRACT

The invention provides fusion proteins comprising at least one fluorescent protein that is linked to at least one transporter protein that changes three-dimensional conformation upon specifically transporting its substrate. The transporter protein may be a nitrate transporter, a peptide transporter, or a hormone transporter. The invention provides fusion proteins comprising at least one fluorescent protein that is linked to at least one mechanosensitive ion channel protein. The invention also provides for methods of using the fusion proteins of the present invention and nucleic acids encoding the fusion proteins.

### STATEMENT REGARDING FEDERALLY SPONSORED RESEARCH OR DEVELOPMENT

Part of the work performed during development of this invention utilized U.S. Government funds through National Science Foundation Grant No. MCB-1021677. The U.S. Government has certain rights in this invention.

### SEQUENCE LISTING INFORMATION

A computer readable text file, entitled “056100-5096-US-SequenceListing.txt,” created on or about Nov. 6, 2014 with a file size of about 117 kb, contains the sequence listing for this application and is hereby incorporated by reference in its entirety.

### BACKGROUND OF THE INVENTION

1. Field of the Invention

The invention provides fusion proteins comprising at least one fluorescent protein that is linked to at least one transporter protein that changes three-dimensional conformation upon specifically transporting its substrate. The invention also provides fusion proteins comprising at least one fluorescent protein that is linked to at least one mechanosensitive ion channel protein. The invention also provides for methods of using the fusion proteins of the present invention and nucleic acids encoding the fusion proteins.

2. Background of the Invention

Transporter proteins play key roles in the physiology of all organisms. They control what enters and leaves the cell and the subcellualr compartments. Mutations in transporter genes are the underlying cause for various human diseases. (Sahoo et al., Front. Physiol., 5: 91, ecollection (2014)).

Transporter proteins play roles such as surface receptors for viral infection and are involved in various diseases. One example is the roles played by the SWEET sugar transporters in pathogen resistance. (Chen et al., Nature, 468, 527-532 (2014)). Transporter proteins are also key to drug action—if they transport the drug efficiently to the intended site of action the drugs will have high efficacy, if they transport the drug to the wrong site (cell type or organ), this can lead to negative side effects (Giacomini et al., Nature Reviews Drug Discovery, 9, 215-236 (2010); Amidon G L, Pharmaceutical Biotechnology, (1999)).

Transporters require complicated technologies to measure their activity. Radiotracers have the disadvanatage of negative side effects and the inability to trace their metabolism. Often metabolism is measured as an indirect indicator of activity of a transporter. Thus, a rapid test of activity is required that is generalizable. Such tests are of particular importance for measuring transporter activities that take place deep inside tissues or at local sites of a cell or within a compartment. For example, transport across the Golgi membrane or vacuole cannot be measured without invasive approaches. Measurements in these cases are out of context since purification of organelles or compartments leads to loss of content and eliminates natural environment. Also, while GFP or similar fusions can indicate where a transporter is, we often do not know when and where the substrate is, or how the transporter is regulated, e.g. by phosphorylation, so we need tools to monitor the activity of the transporter in vivo.

As indicated, a major limitation of the classical biosensor techniques is that such techniques are not applicable to intact living tissues and have limited spatial and temporal resolution. An alternative approach for such analysis has been the engineering of promoter-reporter constructs sensitive to nitrate concentration changes. These constructs have been useful, but they are limited by the indirect nature of the reporters and the limited spatial and temporal resolution. Reports are delayed, often influenced by other signals integrated by the promoter elements, and kinetics are affected e.g. by RNA stability or translation efficiency. For example, one of the primary problems is that promoters are subject to multiple inputs and that there is a large delay between a change and a report. The stability of RNA and protein also affects the readout, thus if the promoter is inducible, the indicator signal will decay slowly when the local concentration of substrate drops.

Accordingly, there is a need for biosensors that can measure the activity of proteins in vivo, as well as the presence or absence of nitrate and/or peptides in living systems and in experimental settings. For example, if a gene for a specific transporter is known, one can look at transcriptional regulation and can produce the protein in heterologous system, study its properties and even study posttranslational regulation. One can label the protein with a fluorphore, e.g., a fluorescent protein, to detect its cellular localization as well as posttranslational effects such as residence time in the membrane, regulated endocytosis etc. These transporters, however, can only “work” in the presence of their substrates or ligands. But even if the ligand is present in sufficient amounts and the protein is in the correct cellular compartment, e.g., the plasma membrane to allow import or export of a given substrate, the protein can be in an inactive state. The ammonium transporter AMT for example is regulated negatively through posttranslational modification and allosteric inactivation of the trimeric transporter complex (Logue, et al., Nature, 446, 195-98 (2007); Lanquar, et al., Plant Cell 21, 3610-22 (2009)). The potassium channel AKT1 in Arabidopsis has to be activated by a kinase, otherwise it may be present, but inactive (Ren, et al., Plant J. 74:258-66 (2013)). Also, the activity state of enzymes and transporters is known to be monitored by the cell itself. Overexpression and repression of sucrose phosphate synthase (SPS) had little effect on sucrose transport, because the cell monitors SPS activity and adjusts its activity according to its needs. When additional SPS protein was present in experimental settings, the cell inactivated part of the protein, when there was less, more active enzyme was generated and phosphorylated (Toroser et al., Plant J. 17:407-13 (1999)). These are three examples of many, which highlight that knowledge of the gene expression and the localization of the protein are valuable but insufficient information to judge whether and how active a given protein is in the cell. Thus there is an apparent need to know where substrates are, when and where the transporter protein is present, and also when the protein is functioning. Quantitative data on the in vivo activity is also needed. In addition, new tools could be helpful in monitoring the effect of a drug in vivo, e.g. a mouse model or cell lines. Drug screens and analysis of side effects can be explored using a tool that can measure the activity of a transporter in vivo.

Many transporter proteins will function only when placed in the proper environment, when it is activated (or derepressed), and when substrate is present. In a multicellular organism, however, it is currently not possible to know the concentration of the substrate, e.g. nitrate, peptide or hormone, at the membrane where the transporter is present, thus tools are needed to measure the activity of the transporter in vivo. Thus, even though genetic analysis can be used to localize specific proteins, and, by extension its substrates, this information may not be useful or helpful if the protein is not active.

The novel fusion proteins of the present invention allow one to study the activity state of the transport or mechanosensitivity in vivo in specific cells of interest, for example the endodermis of the root or the blood brain barrier as two out of many examples. One family of proteins (named NPF) targeted here (Léran et al., Trends Plant Sci., September 18. doi:pii: S1360-1385 (2013)) is of particular interest since members of this family have been shown to transport other important substrates, such as plant hormones, secondary metabolites and drugs (Kanno et al., Proc Nat'l Acad Sci USA 109:9653-8 (2012); Mounier et al., Plant Cell Environ. June 3. doi: 10.1111/pce.12143 (2013); Newstead, Biochem Soc Trans. 39:1353-8 (2011); Anderson and Thwaites Physiology 25:364-77 (2010)). These proteins are important for hormone and nitrogen homeostasis as well as for metazoan and human nutrition. They also are important in the context of inflammatory diseases (Ingersoll et al., Am J Physiol Gastrointest Liver Physiol. 302:G484-92 (2012); Rubio-Aliaga and Daniel Xenobiotica. 38:1022-42 (2008)).

### SUMMARY OF THE INVENTION

The invention provides fusion proteins comprising at least one fluorescent protein that is linked to at least one transporter protein that changes three-dimensional conformation upon specifically transporting its substrate or at least reporting conformational changes that occur during the transport cycle as a proxy for its activity or the available substrate levels. The invention also provides fusion proteins comprising at least one fluorescent protein that is linked to at least one mechanosensitive ion channel protein. The invention also provides for methods of using the fusion proteins of the present invention and nucleic acids encoding the fusion proteins.

The invention also provides for methods of measuring nitrate, peptide or hormones in a sample, comprising contacting the sample with a fusion protein present in a cell or membrane compartment of the present invention.

The present invention also provides for nucleic acids encoding the fusion proteins of the present invention.

### BRIEF DESCRIPTION OF THE DRAWINGS

FIG. 1 depicts (A) the cDNA sequence of NRT1.1 (CHL1) from Arabidopsis thaliana, (B) the translated amino acid sequence of NRT1.1 (CHL1) from Arabidopsis thaliana, (C) the amino acid sequence of PTR1 from Arabidopsis thaliana, (D) the amino acid sequence of PTR2 from Arabidopsis thaliana, (E) the amino acid sequence of PTR4 from Arabidopsis thaliana and (F) the amino acid sequence of PTR5 from Arabidopsis thaliana, (G) the cDNA sequence of PTR1 from Arabidopsis thaliana, (H) the cDNA sequence of PTR2 from Arabidopsis thaliana, (I) the cDNA sequence of PTR4 from Arabidopsis thaliana and (J) the cDNA sequence of PTR5 from Arabidopsis thaliana.

FIG. 2 depicts quenching of the fluorophores of one of the fusion proteins of the present invention in response to nitrate transport. The nitrate transporter protein in this embodiment is wild-type Arabidopsis thaliana NRT1.1. This construct (FLIP 30) comprises fluorophores of this particular fusion protein are mCFP fused to the C-terminal of NRT1.1 and AFPt9 fused to the N-terminus. A, C show that the quenching is nitrate specific. B shows the FRET emission ration over a range of wavelengths. D shows FRET emission at a single wavelength.

FIG. 3 depicts the response of a fusion protein comprising a mutant NRT1.1 protein in which the “high affinity” response of the nitrate transporter protein has been ablated by mutating the threonine at position 101 of Arabidopsis thaliana NRT1.1 to alanine (the low affinity mutant of NRT1.1). The sensor does not respond to addition of low levels of KNO₃.

FIG. 4 depicts the response of a fusion protein comprising a mutant NRT1.1 protein in which the “high affinity” response of the nitrate transporter protein has been ablated by mutating the threonine at position 101 of Arabidopsis thaliana NRT1.1 to alanine (the low affinity mutant of NRT1.1). The sensor only responds to addition of high levels of KNO₃.

FIG. 5 depicts a construct of the present invention comprising the CHL1 nitrate transporter and two fluorophores. The construct (Aphrodite-t9 fused to the N-terminus of CHL1 and Teal-t9 fused to the C-terminus) displays FRET between the two fluorophores, but addition of nitrate does not induce a change in FRET.

FIG. 6 depicts another construct of the present invention comprising the CHL1 nitrate transporter and two fluorophores at different positions than the construct in FIG. 5. The construct (AFPt9 fused to the central loop of CHL1 and Teal-t9 fused to loop between transmembrane helices 10 and 11) displays FRET between the two fluorophores, but addition of nitrate does not induce a change in FRET.

FIG. 7 depicts quenching of the fluorophores of one of the fusion proteins of the present invention in response to nitrate transport. The nitrate transporter protein in this embodiment is wild-type Arabidopsis thaliana NRT1.1. This construct (FLIP 39) comprises fluorophores of this particular fusion protein are t7sCFPt9 fused to the C-terminal of NRT1.1 and AFPt9 fused to the N-terminus.

FIG. 8 depicts quenching of the fluorophores of one of the fusion proteins of the present invention in response to nitrate transport. The nitrate transporter protein in this embodiment is wild-type Arabidopsis thaliana NRT1.1. This construct (FLIP 42) comprises fluorophores of this particular fusion protein are mCFP fused to the C-terminal of NRT1.1 and Citrine fused to the N-terminus.

FIG. 9 depicts FRET between two fluorophores of one of the fusion proteins of the present invention in response to di-peptide (A, Gly-GLy; B, Ala-Leu) transport. (A) The peptide transporter protein in this embodiment is wild-type Arabidopsis thaliana PTR4. This construct (FLIP 39) comprises fluorophores of this particular fusion protein are t7sCFPt9 fused to the C-terminal of PTR4 and AFPt9 fused to the N-terminus. (B) The peptide transporter protein in this embodiment is wild-type Arabidopsis thaliana PTR4. This construct (FLIP 39) comprises fluorophores of this particular fusion protein are t7sCFPt9 fused to the C-terminal of PTR4 and AFPt9 fused to the N-terminus.

FIG. 10 depicts operation of the sensor of the construct shown in FIG. 2 with putative interactors. These interactors potentially interact (augment or interfere with) in vivo nitrate transport. Their interaction can be visualized by addition of the substrate, in this case KNO₃, with candidate interactor compounds.

FIG. 11 depicts quenching of the fluorophores of one of the fusion proteins of the present invention in response to nitrate transport. The peptide transporter protein in this embodiment is wild-type Arabidopsis thaliana PTR5. This construct (FLIP 39) comprises fluorophores of this particular fusion protein are t7sCFPt9 fused to the C-terminal of PTR and AFPt9 fused to the N-terminus. A-E depict quenching in response to transport of various substrates.

FIG. 12 depicts quenching or/and FRET between two fluorophores of the fluorophores of one of the fusion proteins of the present invention in response to nitrate transport. The nitrate transporter proteins in this embodiment are wild-type Arabidopsis thaliana NRT1.1 and different individual mutant constructs of CHL1 (E41A, E44A, R45A, T48A, L49A, K164A, K164R, H356A, 0358A, Y388A, Y388F, E476A, and E476D). This construct (pDRFLIP 30) comprises the fluorophores of the construct shown in FIG. 2 with CHL1.

FIG. 13 depicts that the kinetics of NiTrac1 and the mutated form of NiTrac1-T101A are biphasic and the affinities of the two phases for both NiTrac1 and the mutant are surprisingly similar to the ones measured by Liu, K and Tsay, Y, (EMBO J., 22(5):1005-1013 (2003), hereby incorporated by reference) for the transporter and the mutant when expressed in Xenopus oocytes.

FIG. 14 depicts quenching of the signal fluorophore of one of the fusion proteins of the present invention in response to nitrate transport. The nitrate transporter protein in this embodiment is wild-type Arabidopsis thaliana NRT1.1. This construct pDRFlip301 (SEQ ID NO: 17) comprises signal fluorophore of this particular fusion protein are mCerulean fused to the C-terminal of NRT1.1.

FIG. 15 depicts quenching and enhancing (inset panel) of the fluorophores of one of the fusion proteins of the present invention in response to nitrate transport. The nitrate transporter protein in this embodiment is wild-type Arabidopsis thaliana NRT1.1. This construct pDRFlip303 (SEQ ID NO: 19) comprises fluorophores of this particular fusion protein are mCerulean fused to the C-terminal of NRT1.1 and mKate2 fused to the N-terminus.

FIG. 16 depicts another construct of the present invention comprising the CHL1 nitrate transporter and two fluorophores swapping positions than the construct in NiTrac1. The construct pDRFlip302 (SEQ ID NO: 18) comprises fluorophores of this particular fusion protein are AFPt9 fused to the C-terminal of NRT1.1 and mCerulean fused to C-terminal of NRT1.1) displays addition of nitrate does not induce a change in FRET.

FIG. 17 depicts FRET between two fluorophores of one of the fusion proteins of the present invention in response to Auxin (IAA) transport. The auxin transporter protein in this embodiment is wild-type Arabidopsis thaliana PIN2. This construct (FLIP 39) (pDRFlip391-PinTrac1; SEQ ID NO: 20) comprises fluorophores of this particular fusion protein are t7sCFPt9 fused to the C-terminal of PIN2 and AFPt9 fused to the N-terminus.

FIG. 18 depicts FRET between two fluorophores of one of the fusion proteins of the present invention in response to Auxin (IAA) transport. The auxin transporter protein in this embodiment is wild-type Arabidopsis thaliana PIN1. This construct (FLIP 391) comprises fluorophores of this particular fusion protein are t7sCFPt9 fused to the C-terminal of PIN1 and AFPt9 fused to the N-terminus.

FIG. 19 depicts the kinetics of the Auxin uptake kinetics of PIN2 as determined with the fluorescence response kinetics of the PinTrac2 sensor.

FIG. 20 depicts emission spectrum of the OzTrac-MSL10 expressed in yeast cells; excitation at 440 nm. Addition 1M NaCl leads to decrease in fluorescence intensity of donor and increase of acceptor.

FIG. 21 depicts Addition of 1M osmolytes including NaCl, KCl, sorbitol, glucose and glycerol leads to higher FRET emission ratio (peak fluorescence intensity of Aphordite excited at 505 nm over emission intensity at 490 nm obtained with excitation at 440 nm).

FIG. 22 depicts emission spectrum of the OzTrac-MSL10 expressed in yeast cells; excitation at 440 nm. Addition of serial NaCl concentrations (mM) resulted in concentration-dependent FRET changes.

FIG. 23 shows the sequence (SE ID NO: 17) and structure of pDRFlip301.

FIG. 24 shows the sequence (SE ID NO: 18) and structural of pDRFlip302.

FIG. 25 shows the sequence (SE ID NO: 19) and structural of pDRFlip303.

FIG. 26 shows the sequence (SE ID NO: 20) and structural of pDRFlip391-PinTrac1.

### DETAILED DESCRIPTION OF THE INVENTION

The invention provides fusion proteins comprising at least one fluorescent protein that is linked to at least one transporter protein that changes three-dimensional conformation upon specifically transporting its substrate. The invention also provides fusion proteins comprising at least one fluorescent protein that is linked to at least one mechanosensitive ion channel protein. The invention also provides for methods of using the fusion proteins of the present invention and nucleic acids encoding the fusion proteins. The fusion proteins of the present invention may or may not be isolated.

The terms “peptide,” “polypeptide” and “protein” are used interchangeably herein. As used herein, an “isolated polypeptide” is intended to mean a polypeptide that has been completely or partially removed from its native environment. For example, polypeptides that have been removed or purified from cells are considered isolated. In addition, recombinantly produced polypeptides molecules contained in host cells are considered isolated for the purposes of the present invention. Moreover, a peptide that is found in a cell, tissue or matrix in which it is not normally expressed or found is also considered as “isolated” for the purposes of the present invention. Similarly, polypeptides that have been synthesized are considered to be isolated polypeptides. “Purified,” on the other hand is well understood in the art and generally means that the peptides are substantially free of cellular material, cellular components, chemical precursors or other chemicals beyond, perhaps, buffer or solvent. “Substantially free” is not intended to mean that other components beyond the novel peptides are undetectable. The fusion proteins of the present invention may be isolated or purified.

As used herein, the term fusion protein is, generally speaking, used as it is in the art and means two peptide fragments covalently bonded to one another via a typical amine bond between the fusion partners, thus creating one contiguous amino acid chain.

The fusion proteins of the present invention comprise at least one fluorescent protein. In one embodiment, however, fusion proteins of the present invention comprise at least two different fluorescent proteins. As used herein, fluorescent proteins are determined to be “different” from one another by the wavelength of light that each protein emits. For example, two “different” fluorescent proteins as used herein will emit light at wavelengths that are different from one another. The invention also contemplates fusion proteins with more than two fluorescent proteins. For example, the fusion proteins of the present application may comprise three, four, five or even six fluorescent proteins, with at least two of the fluorescent proteins being different from one another. Of course, each of the two or more fluorescent proteins may be different from one another, as defined herein.

The term “fluorescent protein” is readily understood in the art and simply means a protein that emits fluorescence at a detectable wavelength. Examples of fluorescent proteins that are part of fusion proteins of the current invention include, but are not limited to, green fluorescent proteins (GFP, AcGFP, ZsGreen), red-shifted GFP (rs-GFP), red fluorescent proteins (RFP, including DsRed2, HcRed1, dsRed-Express, cherry, tdTomato), yellow fluorescent proteins (YFP, Zsyellow), cyan fluorescent proteins (CFP, AmCyan), AFP, AFPt9 a blue fluorescent protein (BFP), amertrine, citrine, cerulean, mCerulean, mKate2, t7sCFPt9, turquoise, VENUS, teal fluorescent protein (TFP), LOV (light, oxygen or voltage) domains, and the phycobiliproteins, as well as the enhanced versions and mutations of these proteins. Table I below provides a non-exhaustive list of examples of fluorescent proteins that may be used in the compositions and methods of the present invention. Fluorescent proteins as well as enhanced versions thereof are well known in the art and are commercially available. For some fluorescent proteins, “enhancement” indicates optimization of emission by increasing the protein's brightness, creating proteins that have faster chromophore maturation and/or alteration of dimerization properties. These enhancements can be achieved through engineering mutations into the fluorescent proteins.

TABLE I
Table of Fluorescent Proteins

| Abbreviation | Full name | Notes |
|---|---|---|
| VFP | Venus | Yellow |
| AFP | Aphrodite | Yellow (codon changed Venus) |
| ChFP | mCherry | Red |
| TFP | mTeal | Blue |
| CFP | eCyan | Blue |
| Cit | Citrine | Yellow |
| Cer | Cerulean | Blue |
| AcGFP | Green | Green |
| Tom | Tomato | Orange/red |
| Ame | Ametrine | Green/yellow |
| Trq | Turquoise | Blue |
| td | tandem dimer | brighter variant |
| s | sticky | dimer tendency variant |
| m | monomeric | dimer tendency variant |
| t# | truncation | N- or C- terminal |
| w/out s or m | weak dimer | original eGFP |
| x | no fluorophore | useful for intramolecular SMS |

Specific combinations of fluorescent proteins that can be used in combination with the transporter proteins or mechanosensitive ion channel protein of the present invention include but are not limited to: AFP/Cer, AFP/TFP, AFP/CFP, Cit/Cer. Enhanced versions of fluorophores may also be used. For example, AFPt9 (truncation of the nine C-terminal residues of AFP)/TFPt9, AFPt9/t7TFPt9 (truncation of the seven N-terminal residues of TFP and truncation of the nine C-terminal residues of TFP), AFPt9sticky/t7CFPt9 (“AFPt9sticky” is a well-known variant of AFP with a strong tendency towards self dimerization).

The fluorescent proteins, for example the phycobiliproteins, may be particularly useful for creating tandem dye labeled labeling reagents. In one embodiment of the current invention, therefore, the measurable signal of the fusion protein is actually a transfer of excitation energy (resonance energy transfer) from a donor molecule (e.g., a first fluorescent protein) to an acceptor molecule (e.g., a second fluorescent protein). In particular, the resonance energy transfer is in the form of fluorescence resonance energy transfer (FRET). When the fusion proteins of the present invention utilize FRET to measure or quantify analyte(s), one fluorescent protein of the fusion protein construct can be the donor, and the second fluorescent protein of the fusion protein construct can be the acceptor. The terms “donor” and “acceptor,” when used in relation to FRET, are readily understood in the art. Namely, a donor is the molecule that will absorb a photon of light and subsequently initiate energy transfer to the acceptor molecule. The acceptor molecule is the molecule that receives the energy transfer initiated by the donor and, in turn, emits a photon of light. The efficiency of FRET is dependent upon the distance between the two fluorescent partners and can be expressed mathematically by: E=R₀⁶/(R₀⁶+r⁶), where “E” is the efficiency of energy transfer, “r” is the distance (in Angstroms) between the fluorescent donor/acceptor pair and “R₀” is the Forster distance (in Angstroms). The Forster distance, which can be determined experimentally by readily available techniques in the art, is the distance at which FRET is half of the maximum possible FRET value for a given donor/acceptor pair. A particularly useful combination is the phycobiliproteins disclosed in U.S. Pat. Nos. 4,520,110; 4,859,582; 5,055,556, incorporated by reference, and the sulforhodamine fluorophores disclosed in U.S. Pat. No. 5,798,276, or the sulfonated cyanine fluorophores disclosed in U.S. Pat. Nos. 6,977,305 and 6,974,873; or the sulfonated xanthene derivatives disclosed in U.S. Pat. No. 6,130,101, incorporated by reference and those combinations disclosed in U.S. Pat. No. 4,542,104, incorporated by reference.

The fusion proteins also comprise at least one transporter protein or a mechanosensitive ion channel protein linked to at least one fluorescent protein. The linkage between the fluorescent protein and the transporter protein or mechanosensitive ion channel protein can be anywhere in the amino acid sequence of the transporter protein. For example, the fluorescent protein may be linked to the N-terminus or C-terminus of the transporter protein or mechanosensitive ion channel protein. In another example, if two fluorescent proteins are used in the fusion constructs of the present invention, the first fluorescent protein may be linked to the N-terminus of the transporter protein or the mechanosensitive ion channel protein and the second fluorescent protein may be linked to the C-terminus of the transporter protein or the mechanosensitive ion channel protein.

The one or more fluorescent proteins may be linked to internal sites in the amino acid sequence of the transporter protein or the mechanosensitive ion channel protein as well. For example the nitrate transporter protein CHL1 (SEQ ID NO:2) is a well-characterized protein with 12 transmembrane alpha helices with small peptide loops connecting each helical domain. The internal, cytosolic loop connecting helices 6 and 7 is known as the central loop. See Ho, C., et al., Cell, 138:1184-1194 (2009), which is incorporated by reference. This structural motif appears to be shared with most if not all member of the PTR family of proteins in plants and other species, including but not limited to hPEPT1 and hPEPT2 in humans. In one embodiment, the one or more fluorescent proteins are linked to internal sites, i.e., not the N-terminus or C-terminus, of the transporter protein in the fusion proteins.

In one embodiment of the current invention, the fusion protein comprises a single polypeptide or protein. In another embodiment, the fusion protein comprises more than one transporter protein, with each transporter protein being a separate or distinct polypeptide or protein. As used herein, “a separate protein” does not necessarily mean that the proteins or polypeptides have distinct amino acid sequences. Instead, “a separate protein” for the purposes of the present invention means that the each of the proteins of the construct is structurally independent and generally, but not necessarily, possesses characteristics of small globular proteins. A “distinct protein,” on the other hand is used to mean proteins or polypeptides that have different amino acid sequences, with each protein of the transporter proteins having characteristics of small globular proteins. In specific embodiments, the fusion proteins of the present invention comprise one, two, three, four, five or six transporter proteins.

In one embodiment, when the fusion protein comprises more than one transporter protein or more than one mechanosensitive ion channel protein, the transporter proteins or mechanosensitive ion channel proteins are linked together without a linker peptide such that the C-terminus of one transporter protein is linked via a typical amine bond to the N-terminus of another transporter protein. In another embodiment, when the fusion constructs comprises more than one transporter protein or more than one mechanosensitive ion channel protein, the transporter proteins or mechanosensitive ion channel proteins are linked together with a linker peptide, i.e., “a linker peptide.” As used herein, a linker peptide is a used to mean a polypeptide typically ranging from about 1 to about 120 amino acids in length that is designed to facilitate the functional connection of two transporter proteins into a linked construct. To be clear, a single amino acid can be considered a linker peptide for the purposes of the present invention. In specific embodiments, the linker peptide comprises or in the alternative consists of amino acids numbering 1, 2, 3, 4, 5, 6, 7, 8, 9, 10, 11, 12,13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 residues in length. Of course, the linker peptides used in the fusion proteins of the present invention may comprise or in the alternative consist of amino acids numbering more than 120 residues in length. The length of the linker peptide, if present, may not be critical to the function of the fusion protein, provided that the linker peptide permits a functional connection between the transporter proteins or the mechanosensitive ion channel proteins.

It is unclear how the signals from the fusion protein are being generated. For example, it may be the binding of the transporter to its substrate, or it may be a conformational change that occurs during the transport cycle, or it may be activities related to an ion channel. The transporter proteins may be mostly proton cotransporters, so they exist in an open outward state, and they first bind to a proton or to the substrate. The binding of both triggers conformational changes resulting in the protein's occluded substrate bound state, which then opens inside the cell to release its substrate, typically in an ordered fashion. The transporter then returns via its occluded empty conformation to the outside open conformation. Each of these states represents a different conformational intermediate state. For example, Doki, S. et al., Proceedings Nat'l Acad. Sci., 110(28):11343-8 (2013), which is incorporated by reference, provides an overview of conformational states of transporter proteins. Thus the signal from these fusion proteins could be generated from either a conformational change from substrate binding, or from the sum of multiple changes during the transport cycle. The fact that binding kinetics and transport kinetics are not necessarily the same, but that kinetics similar to transport are observed, suggests that the observed signals are due to the activity of the transporter, i.e., its action rather than just binding. For example, in De Michele, R. et al., eLife 2013:2e00800 (elife.elifesciences.org/content/2/e00800), which is incorporated by reference, discusses using what is known about conformational changes of transporter proteins during their transport cycle to generate sensors. In those cases, it is the conformational change during transport that is measured.

The term “functional connection” in the context of a linker peptide indicates a connection that facilitates folding of the polypeptides of each transporter protein or mechanosensitive ion channel protein into a three dimensional structure that allows the linked fusion polypeptides or mechanosensitive ion channel protein to mimic some or all of the functional aspects or biological activities of the transporter proteins or mechanosensitive ion channel protein. For example, in the case of a nitrate transporter, the linker may be used to create a single-chain fusion of a multi-protein to achieve the desired biological activity of transporting nitrate or to achieve a three dimensional structure that mimics the structure of each of the native transporter proteins. In the case of a mechanosensitive ion channel protein, the linker may be used to create a single-chain fusion of a multi-protein to achieve the desired biological activity of being mechanisensitive or to achieve a three dimensional structure that mimics the structure of each of the native mechanosensitive ion channel protein. The term functional connection also indicates that the linked transporter proteins or mechanosensitive ion channel proteins possess at least a minimal degree of stability, flexibility and/or tension that would be required for the transporter protein or the mechanosensitive ion channel protein to function as desired.

In one embodiment of the present invention, fusion proteins have more than one linker peptide, with the linker peptides comprising or consisting of the same amino acid sequence. In another embodiment, fusion proteins have more than one linker peptide, with the amino acid sequences of the linker peptides being different from one another.

In some embodiments of the present invention, the fusion proteins of the present invention comprise at least one transporter protein, which functions to move molecules within an organism. The transporter proteins used in the present invention may include but not be limited to: nitrate transporters, peptide transporter, or hormone transporter.

In some embodiments, the transporter proteins of the present invention may be members of the solute carrier (SLC) group of membrane transport proteins, which transport charged and uncharged organic molecules as well as inorganic ions and the gas ammonia.

In some embodiments, the transporter proteins of the present invention may be members of the major facilitator superfamily (MFS), which is a class of membrane transport proteins that facilitate movement of small solutes across cell membranes in response to chemiosmotic gradients.

In some embodiments, the transporter proteins of the present invention may be members of the so-called PTR (NRT1) family of transporter proteins or members of the PIN-FORMED (PIN) protein family.

In one embodiment, the transporters are nitrate transporters. Examples of nitrate transporters that are members of the PTR family of nitrate and/or peptide transporters include but are not limited to NRT1.1 (CHL1), NRT1.2, NRT1.3, NRT1.4, NRT1.5, NRT1.6, NRT1.7, NRT1.8, NRT1.9, NRT1.11, NRT1.12, NRT2.1, NRT2.2, NRT2.4 and NRT2.7 proteins and derivatives and mutants thereof. The invention includes all members of the PTR family of transporters. For example, Arabidopsis alone has 53 separate PTR proteins based on genomic sequence analysis, whereas rice has 80 separate PTR proteins based on genomic sequence analysis. Tsay, Y., et al. FEBS Letters, 581:2290-2300 (2007), the entirety of which is incorporated by reference, displays a phylogenetic tree of just the Arabidopsis and rice family members of the PTR family of proteins, and all of these members are included in the scope of the present invention. The term “PTR”(or “NRT”) is used to mean a member of the gene family of PTR transports. In general, “PTR” (or “NRT”) refers to genes and proteins isolated and identified in Arabidopsis thaliana as well as orthologs from other species. For example, the term “NRT1.2” as used herein refers to the NRT1.2 protein or gene from Arabidopsis thaliana as well as the NRT1 protein or gene from Oryza sativa (rice). Thus the invention is not limited to genes and proteins from Arabidopsis thaliana. At least in plants, it appears that nitrate transporters cannot transport peptides and peptide transporters cannot transport nitrates.

Other members of the PTR family of proteins that are useful in the fusion proteins of the present invention include those orthologous members in other species, such as but not limited to PTRs in humans, C. elegans, Drosophila and yeast. For example, the PTR family of proteins is also referred to as proton-dependent oligopeptide transporters (POTs), and the hPEPT family of human transporter proteins belongs to this POT family of proteins. In fact, this POT family of transporters is highly conserved from humans to bacteria. In humans, POT proteins accept almost all di- and tri- peptides but do not transport longer peptides. In addition, these POT proteins transport small peptides such as, but not limited to, beta lactam antibiotics, angiotensin converting enzyme inhibitors and antiviral nucleoside drugs and prodrugs. In one embodiment, the peptide transporter used in the fusion proteins of the present invention are selected from hPEPT1 and hPEPT2, as disclosed in Rubio-Aliaga, I. and Daniel, H., Xenobiotica, 38(7-8):1022-1042 (2008) and incorporated by reference. Of course, the invention also includes orthologs of hPEPT1 and hPEPT2 as the peptide transporter in the fusion proteins of the present invention. The approach described herein has been successfully used for 5 different members of this protein superfamily, thus provising evidenec that this approach can be extended to all members of this superfamily.

In some embodiments of the present invention, the transporter proteins used in the present invention are members of the so-called PIN-FORMED (PIN) protein family. The PIN transporters are responsible for the transport of plant hormone auxin (IAA), which is essentially involved in various processes of plant growth and development. auxin is actively and directionally transported from cell to cell by polar auxin transport. One known transporter protein family facilitating this process is the PIN proteins. Krecek, P. et al, Genome Biology 2009, 10:249, which is entirely incorporated by reference, provides a summary for the structure and function of the PIN protein family. In some embodiments, the fusion proteins of the present invention may be new hormone sensors, particularly for the plant hormone auxin, namely PinTracs based on Arabidopsis PIN1 or PIN2.

Mechanosensitive (MS) ion channels are able to detect osmotic stress. For Example, Haswell, E. et al., Curr Biol. 18(10):730-4 (2008), which is incorporated by references, provides a summary of the mechanisensitive channel small conductance-like proteins as examples of mechanosensitive ion channel proteins. The fusion proteins of the present invention comprising MS ion channels may be used as “osmosensors” that output a fluorescent signal, allowing direct observation of detection of osmotic stress in vivo. In this way, these osmosensors may act as a direct probe with an output that may be measured to monitor the dynamic changes of turgor pressure in vivo.

In some embodiments of the present invention, the fusion proteins of the present invention comprise at least one mechanosensitive ion channel protein. The mechanosensitive (MS) ion channel protein used in the present invention are members of the so-called mechanosensitive small-conductance channel protein family, including but not limited to mechanisensitive channel small conductance-like (MSL) proteins such as MSL10, or more in particular, AtMSL10 (MSL10 from Arabidopsis thaliana). See Nakamura, S. et al. Biosci Biotechnol Biochem 74, 1315-1319 (2010), and Ho, C. H. & Frommer, W. B. eLife 3, e01917 (2014); both references are incorporated in their entirety.

Accordingly, and as used here in some embodiments, the phrase transporter protein is used to mean a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence of NRT or PIN regardless of the source of the protein.

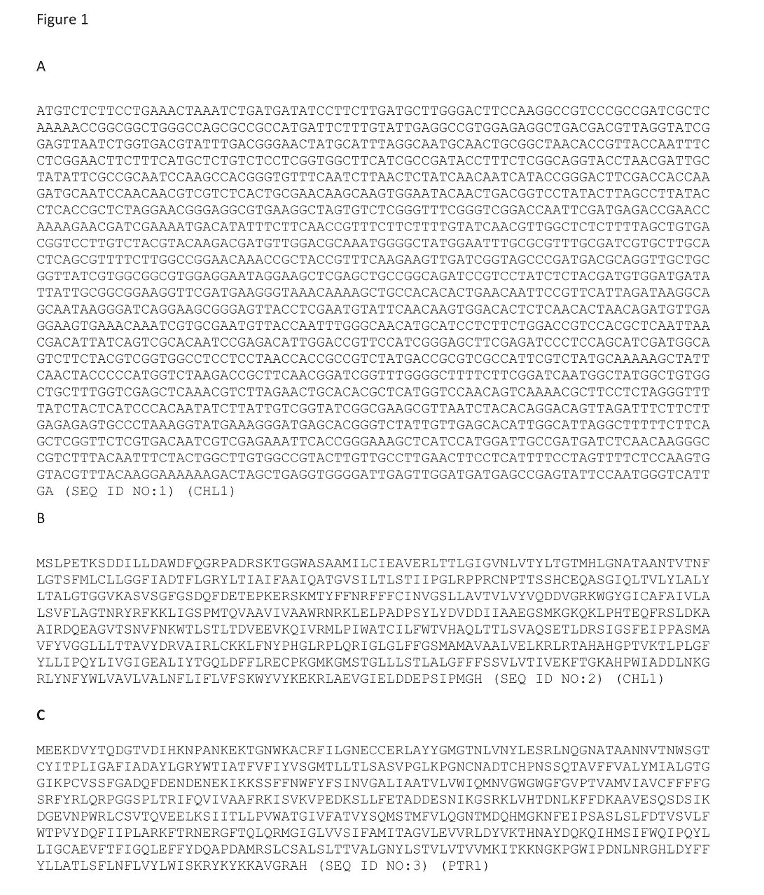

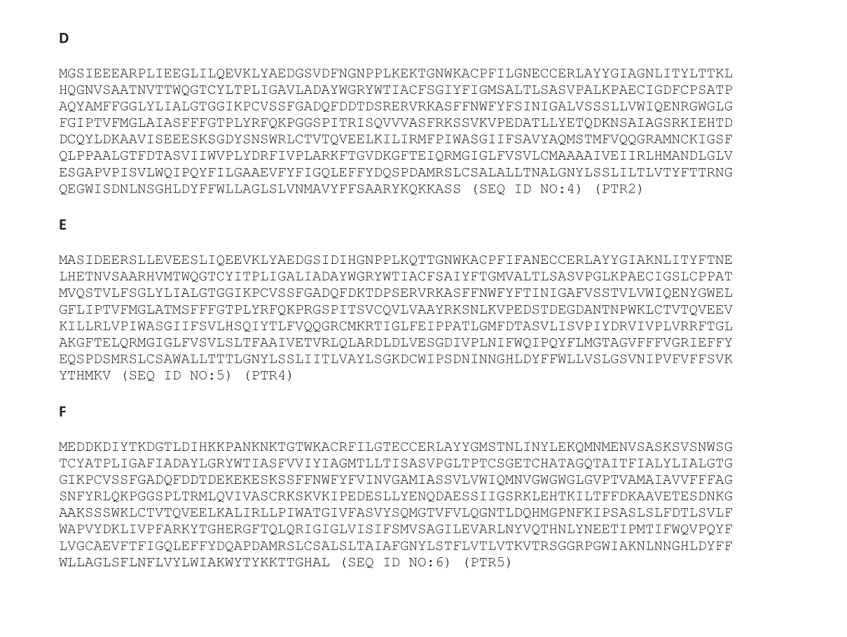

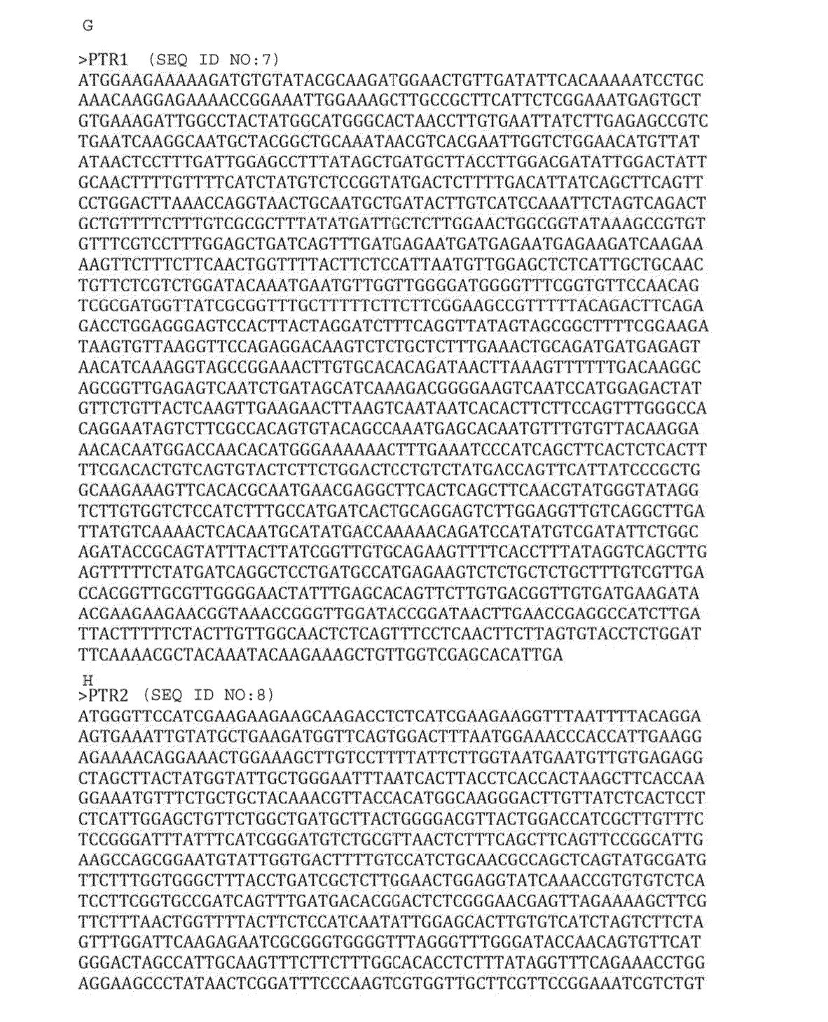

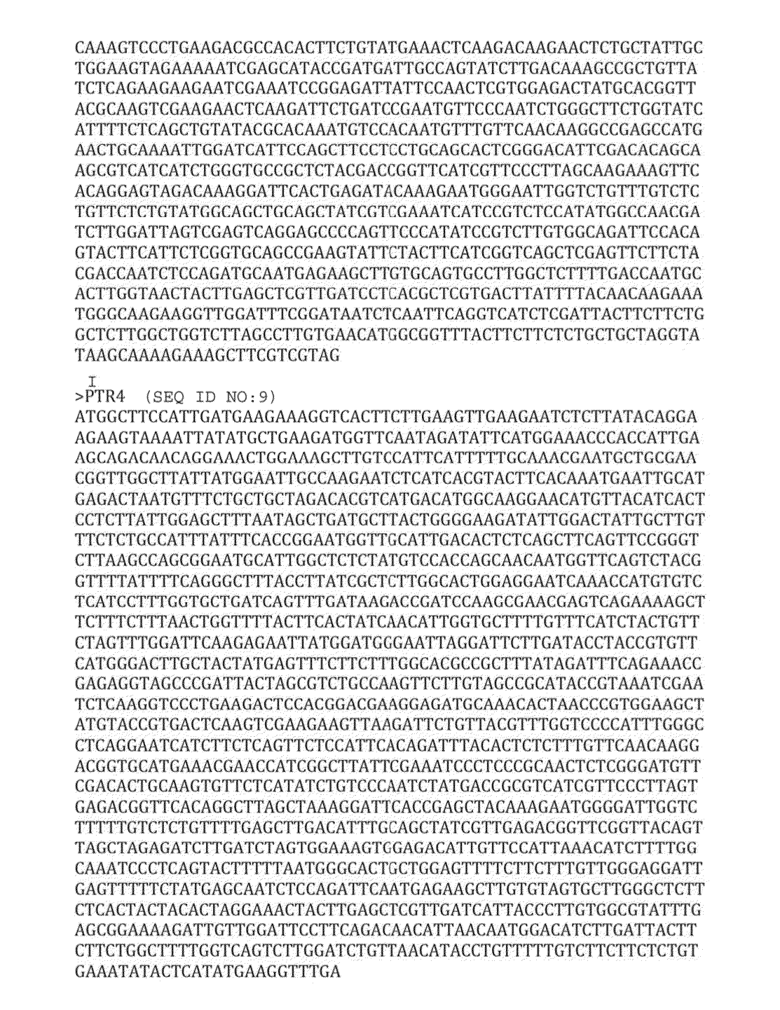

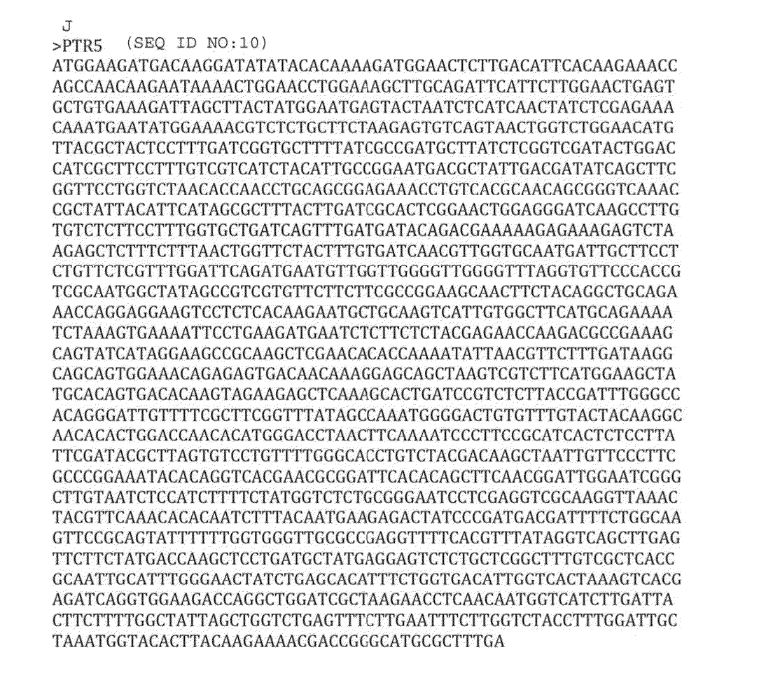

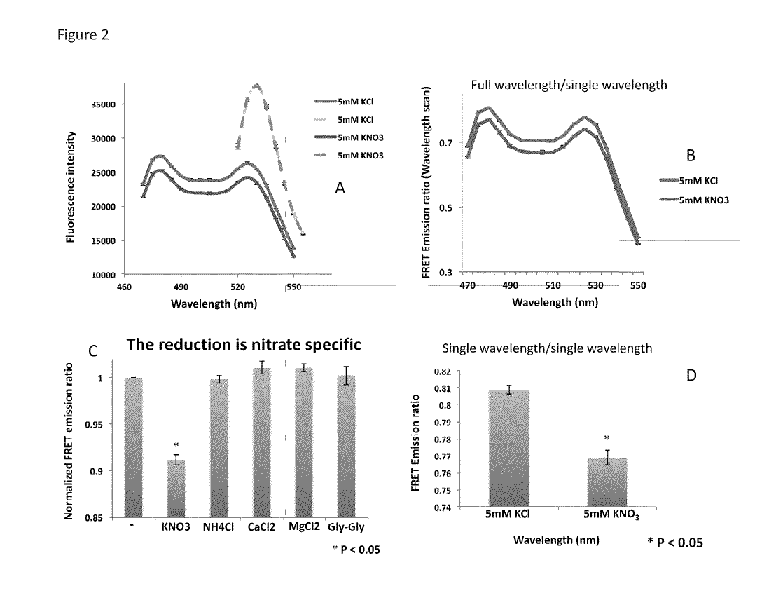

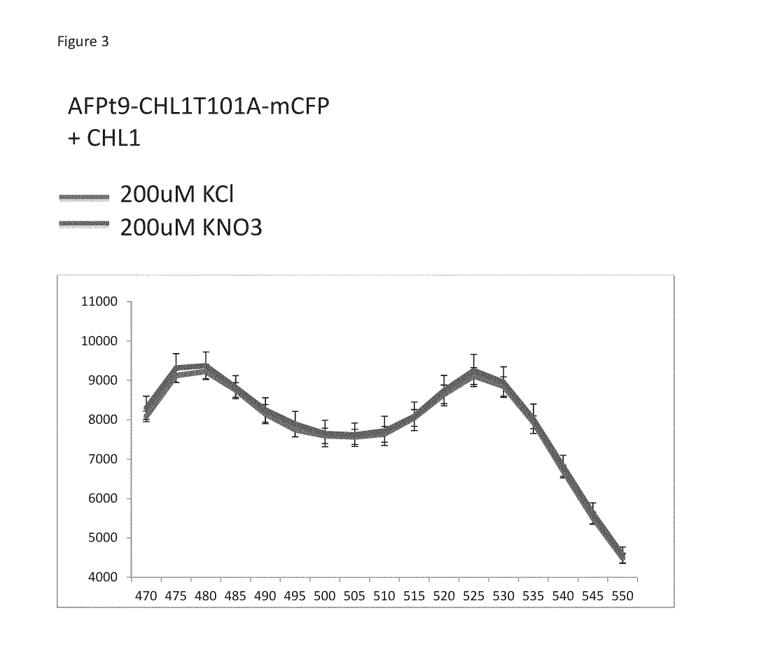

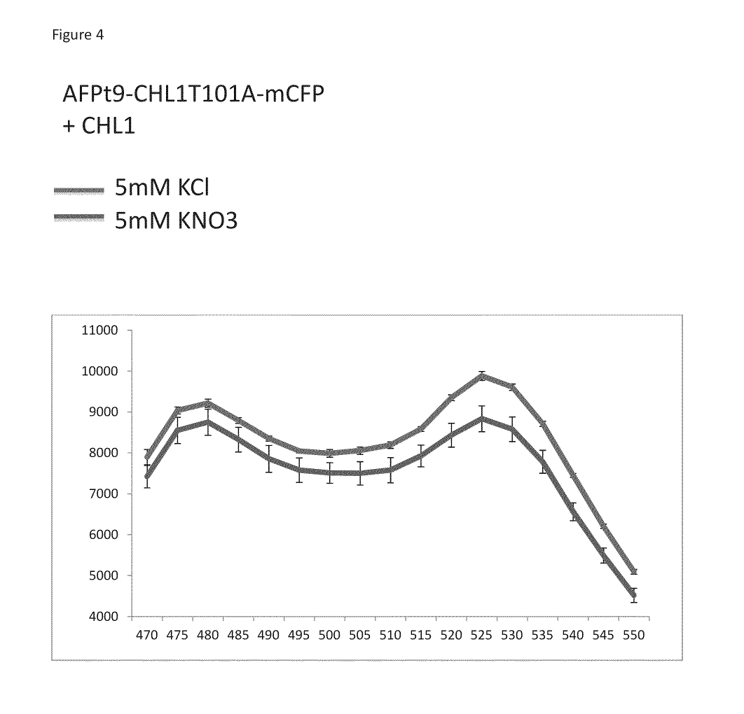

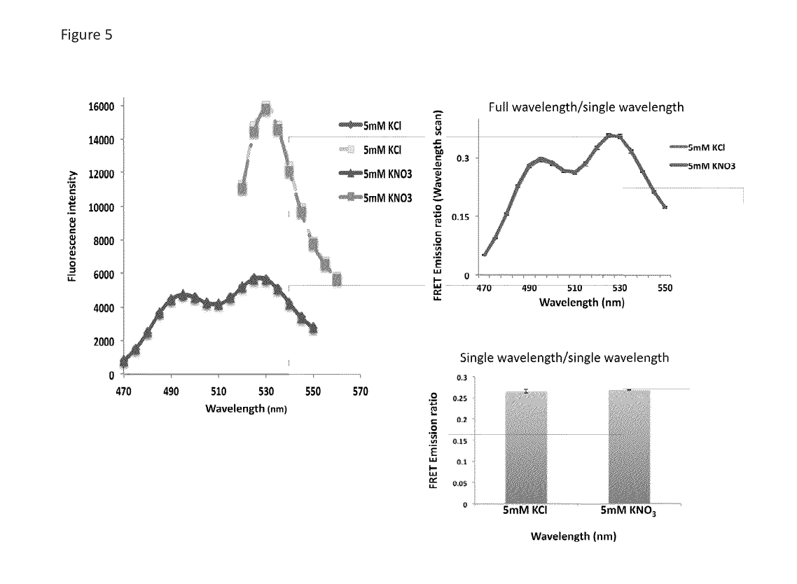

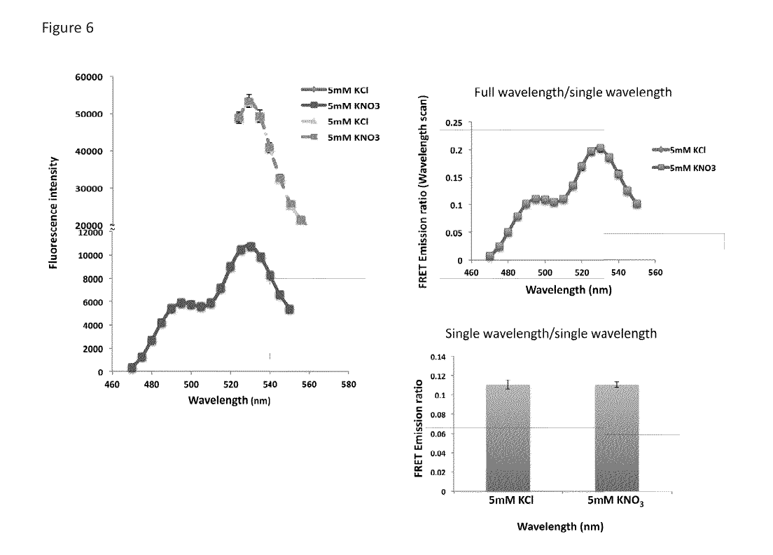

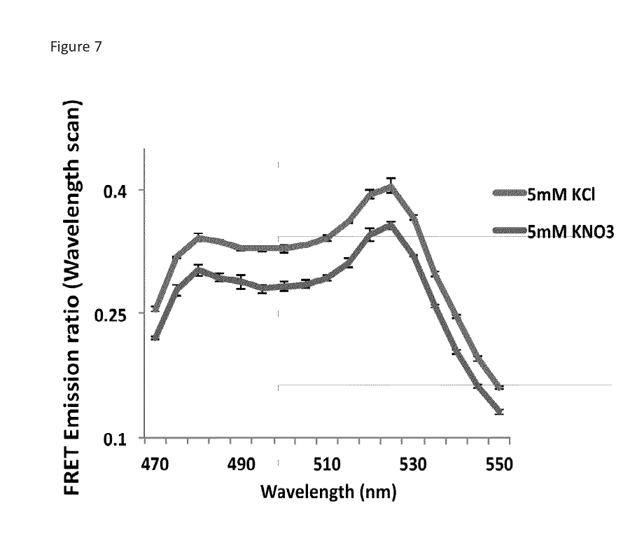

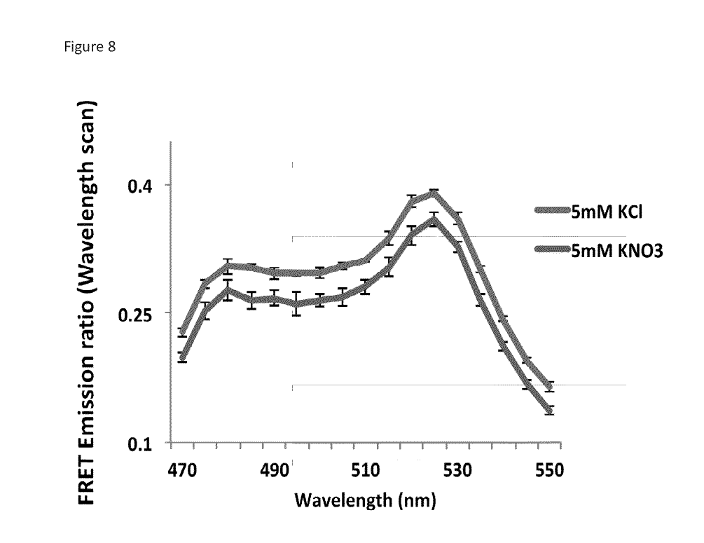

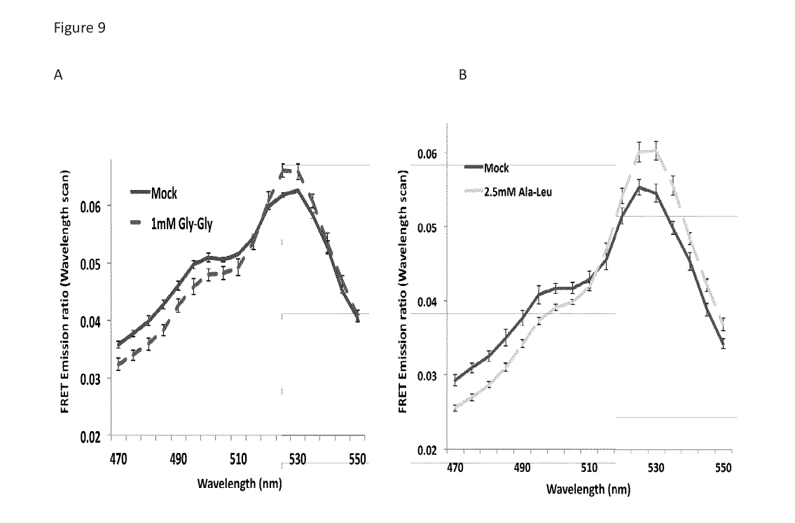

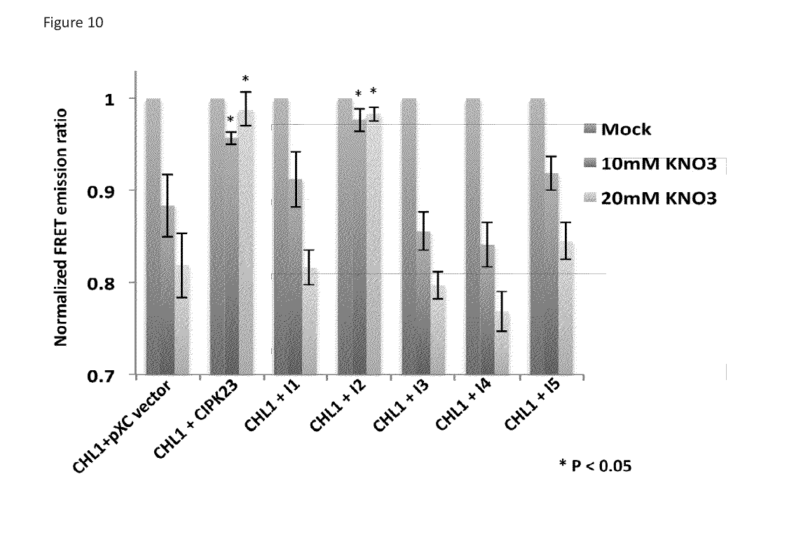

In one embodiment, the transporter protein is a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence in FIG. 1B, (SEQ ID NO:2) (wild-type CHL1 protein of Arabidopsis thaliana). In one embodiment, the transporter protein is a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence in FIG. 1C, (SEQ ID NO:3) (wild-type PTR1 protein of Arabidopsis thaliana). In another embodiment, the transporter protein is a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence in FIG. 1D, (SEQ ID NO:4) (wild-type PTR2 protein of Arabidopsis thaliana). In another embodiment, the transporter protein is a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence in FIG. 1E, (SEQ ID NO:5) (wild-type PTR4 protein of Arabidopsis thaliana). In another embodiment, the transporter protein is a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence in FIG. 1F, (SEQ ID NO:6) (wild-type PTR5 protein of Arabidopsis thaliana).

In one embodiment, the transporter protein is a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence of SEQ ID NO:11 (wild-type PIN1 protein of Arabidopsis thaliana). In one embodiment, the transporter protein is a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence of SEQ ID NO: 14 (wild-type PIN2 protein of Arabidopsis thaliana).

Accordingly, and as used here in some embodiments, the phrase mechanosensitive ion channel protein is used to mean a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence of MSL10 regardless of the source of the protein. In one embodiment, the transporter protein is a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence encoded by SEQ ID NO:22 (AtMLS10 of Arabidopsis thaliana).

A polypeptide having an amino acid sequence at least, for example, about 95% “identical” to a reference an amino acid sequence, e.g., the amino acid sequence of FIG. 1B, is understood to mean that the amino acid sequence of the polypeptide is identical to the reference sequence except that the amino acid sequence may include up to about five modifications per each 100 amino acids of the reference amino acid sequence. In other words, to obtain a peptide having an amino acid sequence at least about 95% identical to a reference amino acid sequence, up to about 5% of the amino acid residues of the reference sequence may be deleted or substituted with another amino acid or a number of amino acids up to about 5% of the total amino acids in the reference sequence may be inserted into the reference sequence. These modifications of the reference sequence may occur at the N-terminus or C-terminus positions of the reference amino acid sequence or anywhere between those terminal positions, interspersed either individually among amino acids in the reference sequence or in one or more contiguous groups within the reference sequence.

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using well known techniques. While there are several methods to measure identity between two polynucleotide or polypeptide sequences, the term “identity” is well known to skilled artisans (Carillo, J. Applied Math. 48, 1073 (1988)). Examples of computer program methods to determine identity and similarity between two sequences include, but are not limited to, GCG program package (Devereux, Nucleic Acids Research 12, 387 (1984)), BLASTP, ExPASy, BLASTN, FASTA (Atschul, J. Mol. Biol. 215, 403 (1990)) and FASTDB. Examples of methods to determine identity and similarity are discussed in Michaels, Current Protocols in Protein Science, Vol. 1, John Wiley & Sons (2011).

In one embodiment of the present invention, the algorithm used to determine identity between two or more polypeptides is BLASTP. In another embodiment of the present invention, the algorithm used to determine identity between two or more polypeptides is FASTDB, which is based upon the algorithm of Brutlag, Comp. App. Biosci. 6, 237-245 (1990)). In a FASTDB sequence alignment, the query and reference sequences are amino sequences. The result of sequence alignment is in percent identity. In one embodiment, parameters that may be used in a FASTDB alignment of amino acid sequences to calculate percent identity include, but are not limited to: Matrix=PAM, k-tuple=2, Mismatch Penalty=1, Joining Penalty=20, Randomization Group Length=0, Cutoff Score=1, Gap Penalty=5, Gap Size Penalty 0.05, Window Size=500 or the length of the subject amino sequence, whichever is shorter.

If the reference sequence is shorter or longer than the query sequence because of N-terminus or C-terminus additions or deletions, but not because of internal additions or deletions, a manual correction can be made, because the FASTDB program does not account for N-terminus and C-terminus truncations or additions of the reference sequence when calculating percent identity. For query sequences truncated at the N- or C-termini, relative to the reference sequence, the percent identity is corrected by calculating the number of residues of the query sequence that are N-and C-terminus to the reference sequence that are not matched/aligned, as a percent of the total bases of the query sequence. The results of the FASTDB sequence alignment determine matching/alignment. The alignment percentage is then subtracted from the percent identity, calculated by the above FASTDB program using the specified parameters, to arrive at a final percent identity score. This corrected score can be used for the purposes of determining how alignments “correspond” to each other, as well as percentage identity. Residues of the reference sequence that extend past the N- or C-termini of the query sequence may be considered for the purposes of manually adjusting the percent identity score. That is, residues that are not matched/aligned with the N- or C-termini of the comparison sequence may be counted when manually adjusting the percent identity score or alignment numbering.

For example, a 90 amino acid residue query sequence is aligned with a 100 residue reference sequence to determine percent identity. The deletion occurs at the N-terminus of the query sequence and therefore, the FASTDB alignment does not show a match/alignment of the first 10 residues at the N-terminus. The 10 unpaired residues represent 10% of the reference sequence (number of residues at the N- and C-termini not matched/total number of residues in the reference sequence) so 10% is subtracted from the percent identity score calculated by the FASTDB program. If the remaining 90 residues were perfectly matched (100% alignment) the final percent identity would be 90% (100% alignment-10% unmatched overhang). In another example, a 90 residue query sequence is compared with a 100 reference sequence, except that the deletions are internal deletions. In this case the percent identity calculated by FASTDB is not manually corrected, since there are no residues at the N- or C-termini of the subject sequence that are not matched/aligned with the query. In still another example, a 110 amino acid query sequence is aligned with a 100 residue reference sequence to determine percent identity. The addition in the query occurs at the N-terminus of the query sequence and therefore, the FASTDB alignment may not show a match/alignment of the first 10 residues at the N-terminus. If the remaining 100 amino acid residues of the query sequence have 95% identity to the entire length of the reference sequence, the N-terminal addition of the query would be ignored and the percent identity of the query to the reference sequence would be 95%.

As used herein, the terms “correspond(s) to” and “corresponding to,” as they relate to sequence alignment, are intended to mean enumerated positions within a reference protein, e.g., wild-type CHL1 from Arabidopsis thaliana, and those positions in, for example, either a modified CHL1 or an orthologous wild-type CHL1 that align with the positions on the reference protein. Thus, when the amino acid sequence of a subject protein is aligned with the amino acid sequence of a reference protein, the amino acids in the subject sequence that “correspond to” certain enumerated positions of the reference sequence are those that align with these positions of the reference sequence, but are not necessarily in these exact numerical positions of the reference sequence. Methods for aligning sequences for determining corresponding amino acids between sequences are described herein.

As used herein, orthologous genes are genes from different species that perform the same or similar function and are believed to descend from a common ancestral gene. Proteins from orthologous genes, in turn, are the proteins encoded by the orthologs. As such the term “ortholog” may be to refer to a gene or a protein. Often, proteins encoded by orthologous genes have similar or nearly identical amino acid sequence identities to one another, and the orthologous genes themselves have similar nucleotide sequences, particularly when the redundancy of the genetic code is taken into account. The art contains information concerning orthologs of genes and proteins. As merely one example, the Uniprot database, found on the world-wide web at www.uniprot.org, contains listings of orthologous proteins.

Accordingly, the transporter protein or portions thereof, or the mechanosensitive ion channel protein or portions thereof, can be from any plant source and the invention is not limited by the source of the transporter, i.e., the invention is not limited to the plant species from which the transporter normally occurs or is obtained. Examples of sources from which the transporter proteins may be derived include but are not limited to monocotyledonous plants that include, for example, Lolium, Zea, Triticum, Sorghum, Triticale, Saccharum, Bromus, Oryzae, Avena, Hordeum, Secale and Setaria. Other sources from which the transporter proteins may be derived include but are not limited to maize, wheat, barley, rye, rice, oat, sorghum and millet. Additional sources from which the transporter proteins may be derived include but are not limited to dicotyledenous plants that include but are not limited to Fabaceae, Solanum, Brassicaceae, especially potatoes, beans, cabbages, forest trees, roses, clematis, oilseed rape, sunflower, chrysanthemum, poinsettia, arabidopsis, tobacco, tomato, and antirrhinum (snapdragon), soybean, canola, sunflower and even basal land plant species, (the moss Physcomitrella patens). Additional sources also include gymnosperms.

In another embodiment, the transporter protein or portion thereof, or the mechanosensitive ion channel protein or portions thereof, can be from any source, including animal cells, bacteria and yeast cells. For example, and as discussed above, the hPEPT proteins are peptide transporter proteins found in animals. These protein transporters function as proton/oligopeptide (including di-peptides and tri-peptides) transporters in the same manner that member of the plant PTR transporters function.

In another aspect, the invention provides deletion variants wherein one or more amino acid residues in the transporter protein, or the mechanosensitive ion channel protein, or one or more fluorescent protein(s) are removed or mutated. Deletions can be effected at one or both termini of the transporter protein or one or more fluorescent protein(s), or with removal of one or more non-terminal amino acid residues of the transporter protein, the mechanosensitive ion channel protein, or one or more fluorescent protein(s).

The fusion proteins of the present invention may also comprise substitution variants of a transporter protein or a mechanosensitive ion channel protein. Substitution variants include those polypeptides wherein one or more amino acid residues of the transporter protein or mechanosensitive ion channel protein are removed and replaced with alternative residues. Examples of substitution variants include but are not limited to a variant in which threonine at amino acid residue 101 of Arabidopsis thaliana NRT1.1 is mutated to either alanine or aspartate (CHL1-T101A and CHL1-T101D, respectively). Of course, the invention encompasses orthologous substitution variants of NRT1.1 at residues that correspond to amino acid position 101 of the Arabidopsis thaliana NRT1.1. Other substitution variants include but are not limited to a P492L mutant of Arabidopsis thaliana NRT1.1 as well as orthologous mutants thereof.

In select embodiments, the fusion proteins of the present invention comprise the NRT1.1 protein and a combination of AFPt9/TFPt9, the NRT1.1 protein and a combination of AFPt9/t7TFPt9, the NRT1.1 protein and a combination of AFPt9sticky/t7CFPt9, the NRT1.1 protein and a mCerulean, the NRT1.1 protein and combination of mCerulean/mKate2, the NRT1.1 protein and a combination of AFPt9/mCerulean. Of course, in any of the above-disclosed embodiments, the NRT1.1 can be from any source. In one embodiment, the NRT1.1 protein in the above-listed fusion proteins is Arabidopsis thaliana NRT1.1 protein. In another embodiment, the NRT1.1 used in the constructs listed above is a mutant construct, more specifically a T101A, a T101D and/or P492L mutant of NRT1.1 from Arabidopsis thaliana (or orthologous mutants of these alanine and arginine mutants at the residues corresponding to the T101 and/or P492 residues of Arabidopsis thaliana).

In select embodiments, the fusion proteins of the present invention comprise the PIN2 protein and a combination of c7sCFPt9/AFPt9, the PIN1 protein and a combination of c7sCFPt9/AFPt9. Of course, in any of the above-disclosed embodiments, the PIN1 or PIN2 can be from any source. In one embodiment, the PIN1 or PIN2 proteins in the above-listed fusion proteins are Arabidopsis thaliana proteins. In another embodiment, the PIN1 or PIN2 used in the constructs listed above are mutant constructs.

In select embodiments, the fusion proteins of the present invention comprise the MSL10 protein and a combination of t7TFPt9/AFPt9. Of course, in any of the above-disclosed embodiments, the MSL10 can be from any source. In one embodiment, the MSL10 protein in the above-listed fusion proteins is AtMSL10. In another embodiment, the AtML10 used in the constructs listed above is a mutant construct.

In one embodiment, the transporter protein or the mechanosensitive ion channel protein is linked to the one or more fluorescent proteins without a linker peptide such that the N-terminus of the transporter protein or the mechanosensitive ion channel protein is linked via a typical amine bond to the C-terminus of one fluorescent protein. In another embodiment, the transporter protein or the mechanosensitive ion channel protein is linked to the one or more fluorescent proteins without a linker peptide such that the C-terminus of the transporter protein or the mechanosensitive ion channel protein is linked via a typical amine bond to the N-terminus of one fluorescent protein. In another embodiment, the transporter protein or the mechanosensitive ion channel protein is linked to the two fluorescent proteins without a linker peptide such that the N-terminus of the transporter protein or the mechanosensitive ion channel protein is linked via a typical amine bond to the C-terminus of one fluorescent protein, and the C-terminus of the transporter protein or the mechanosensitive ion channel protein is linked via a typical amine bond to the N-terminus of another fluorescent protein.

In another embodiment, the transporter protein or the mechanosensitive ion channel protein is linked to one or more fluorescent proteins with a linker peptide, i.e., “a fluorescent protein linker peptide.” In yet another embodiment, the transporter protein or the mechanosensitive ion channel protein is linked to one or more fluorescent proteins with a linker peptide and is linked to the other fluorescent protein without a linker peptide. In the embodiment when only one fluorescent protein linker peptide is used, either the N-terminus or the C-terminus of transporter protein or the mechanosensitive ion channel protein can be the location of the fluorescent protein linker peptide. As used herein, a fluorescent protein linker peptide is used to mean a polypeptide typically ranging from about 1 to about 50 amino acids in length that is designed to facilitate the functional connection of a fluorescent protein to the transporter protein or themechanosensitive ion channel protein. To be clear, a single amino acid can be considered a fluorescent protein linker peptide for the purposes of the present invention. In specific embodiments, the fluorescent protein linker peptide comprises or in the alternative consists of amino acids numbering 1, 2, 3, 4, 5, 6, 7, 8, 9, 10, 11, 12,13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 residues in length. Of course, the fluorescent protein linker peptides used in the fusion proteins of the present invention may comprise or in the alternative consist of amino acids numbering more that 50 residue in length. The length of the fluorescent protein linker peptide, if present, may not be critical to the function of the fusion protein, provided that the fluorescent protein linker peptide permits a functional connection between the fluorescent protein and the transporter protein or the mechanosensitive ion channel protein.

The term “functional connection” in the context of a linker peptide indicates a connection that facilitates folding of the transporter protein or the mechanosensitive ion channel protein and the fluorescent proteins into a three dimensional structure that allows each of the portions of the fusion protein to mimic some or all of the functional aspects or biological activities of the transporter protein or the mechanosensitive ion channel protein and fluorescent protein(s).

In one embodiment of the present invention, the fluorescent protein linker peptide(s) comprise(s) or consist(s) of the same amino acid sequence. In another embodiment, the amino acid sequence(s) of the fluorescent protein linker peptide(s) is(are) different from one another.

In one embodiment of the present invention, the linker peptides that link transporter proteins or the mechanosensitive ion channel protein comprise or consist of the same amino acid sequence as the fluorescent protein linker peptides. In another embodiment, the amino acid sequence of the linker that links transporter proteins or the mechanosensitive ion channel proteins are different from the fluorescent protein linker peptides.

The fusion proteins of the present invention may or may not contain additional elements that, for example, may include but are not limited to regions to facilitate purification. For example, “histidine tags” (“his tags”) or “lysine tags” may be appended to the fusion protein. Examples of histidine tags include, but are not limited to hexaH, heptaH and hexaHN. Examples of lysine tags include, but are not limited to pentaL, heptaL and FLAG. Such regions may be removed prior to final preparation of the fusion protein. Other examples of a second fusion peptide include, but are not limited to, glutathione S-transferase (GST) and alkaline phosphatase (AP).

The addition of peptide moieties to fusion proteins, whether to engender secretion or excretion, to improve stability and to facilitate purification or translocation, among others, is a familiar and routine technique in the art and may include modifying amino acids at the terminus to accommodate the tags. For example the N-terminus amino acid may be modified to, for example, arginine and/or serine to accommodate a tag. Of course, the amino acid residues of the C-terminus may also be modified to accommodate tags. One particularly useful fusion protein comprises a heterologous region from immunoglobulin that can be used to solubilize proteins.

Other types of fusion proteins provided by the present invention include but are not limited to, fusions with secretion signals and other heterologous functional regions. Thus, for instance, a region of additional amino acids, particularly charged amino acids, may be added to the N-terminus of the protein to improve stability and persistence in the host cell, during purification or during subsequent handling and storage.

The fusion proteins of the current invention can be recovered and purified from recombinant cell cultures by well-known methods including, but not limited to, ammonium sulfate or ethanol precipitation, acid extraction, anion or cation exchange chromatography, phosphocellulose chromatography, hydrophobic interaction chromatography, affinity chromatography, e.g., immobilized metal affinity chromatography (IMAC), hydroxylapatite chromatography and lectin chromatography. High performance liquid chromatography (“HPLC”) may also be employed for purification. Well-known techniques for refolding protein may be employed to regenerate active conformation when the fusion protein is denatured during isolation and/or purification.

Fusion proteins of the present invention include, but are not limited to, products of chemical synthetic procedures and products produced by recombinant techniques from a prokaryotic or eukaryotic host, including, for example, bacterial, yeast, higher plant, insect and mammalian cells. Depending upon the host employed in a recombinant production procedure, the fusion proteins of the present invention may be glycosylated or may be non-glycosylated. In addition, fusion proteins of the invention may also include an initial modified methionine residue, in some cases as a result of host-mediated processes.

The invention also relates to isolated nucleic acids and to constructs comprising these nucleic acids. The nucleic acids of the invention can be DNA or RNA, for example, mRNA. The nucleic acid molecules can be double-stranded or single-stranded; single stranded RNA or DNA can be the coding, or sense, strand or the non-coding, or antisense, strand. In particular, the nucleic acids may encode any fusion proteins of the invention. For example, the nucleic acids of the invention include polynucleotide sequences that encode the fusion proteins that contain or comprise glutathione-S-transferase (GST) fusion protein, poly-histidine (e.g., His₆), poly-HN, poly-lysine, etc. If desired, the nucleotide sequence of the isolated nucleic acid can include additional non-coding sequences such as non-coding 3′ and 5′ sequences (including regulatory sequences, for example).

In one embodiment, the nucleic acids of the present invention comprise a polynucleotide sequence that codes for a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence in FIG. 1B, (SEQ ID NO:2) (wild-type CHL1 protein of Arabidopsis thaliana). In another embodiment, the nucleic acids of the present invention comprise a polynucleotide sequence that codes for a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence in FIG. 1C, (SEQ ID NO:3) (wild-type PTR1 protein of Arabidopsis thaliana). In another embodiment, the nucleic acids of the present invention comprise a polynucleotide sequence that codes for a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence in FIG. 1D, (SEQ ID NO:4) (wild-type PTR2 protein of Arabidopsis thaliana). In another embodiment, the nucleic acids of the present invention comprise a polynucleotide sequence that codes for a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence in FIG. 1E, (SEQ ID NO:5) (wild-type PTR4 protein of Arabidopsis thaliana). In another embodiment, the nucleic acids of the present invention comprise a polynucleotide sequence that codes for a protein with an amino acid sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the amino acid sequence in FIG. 1F, (SEQ ID NO:6) (wild-type PTR5 protein of Arabidopsis thaliana).

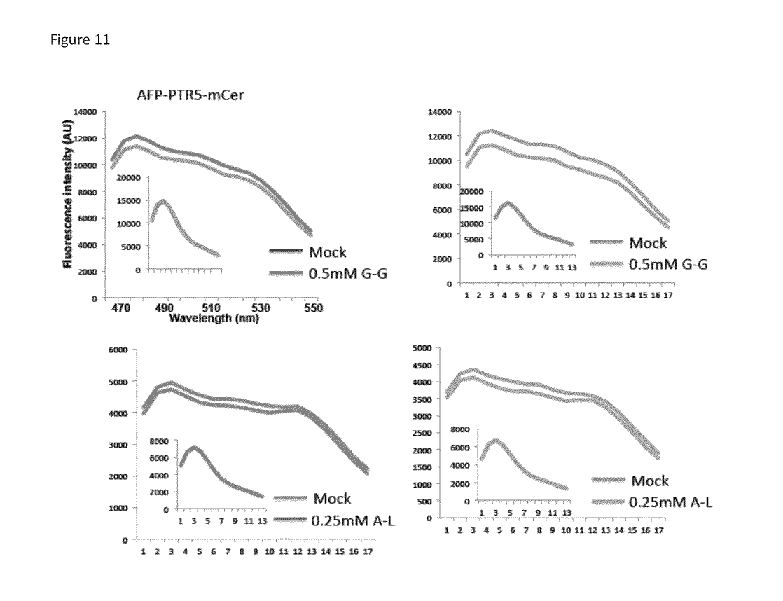

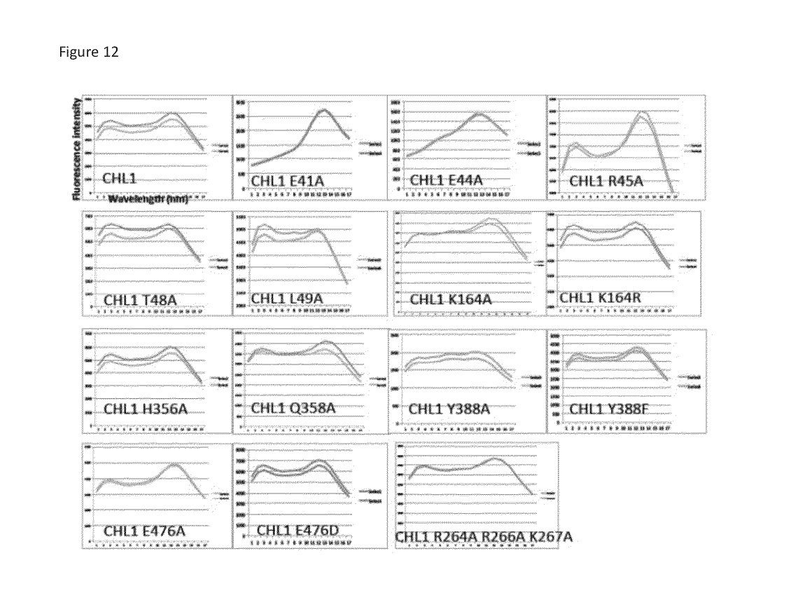

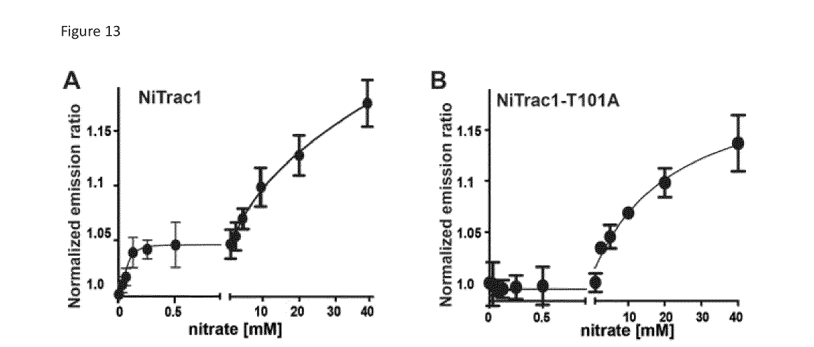

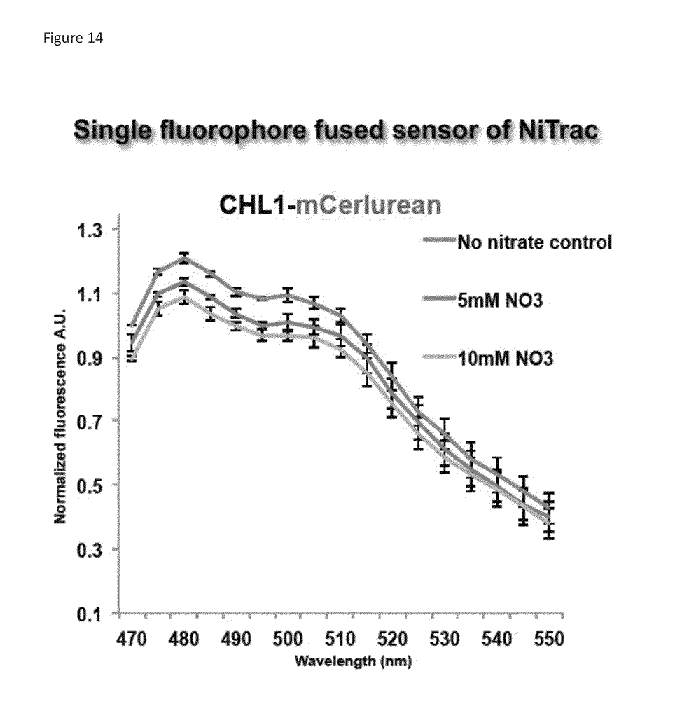

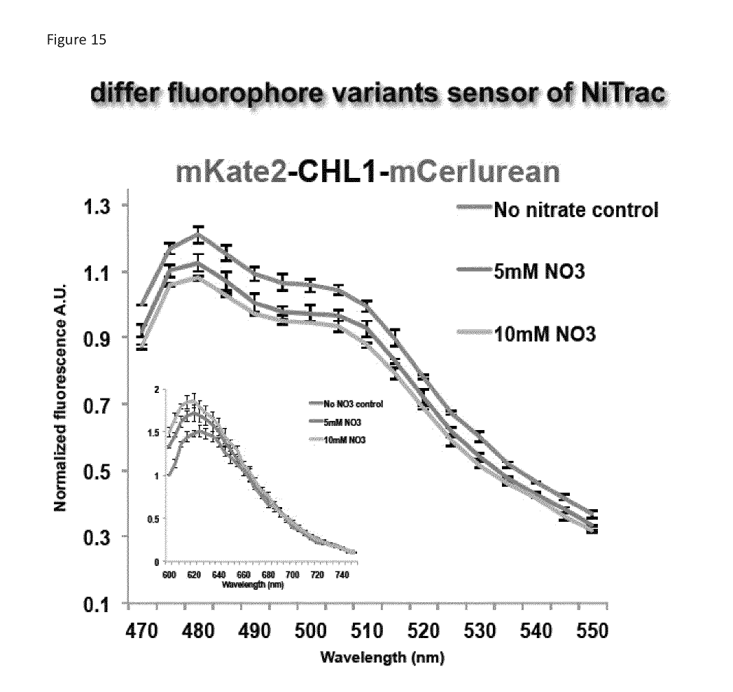

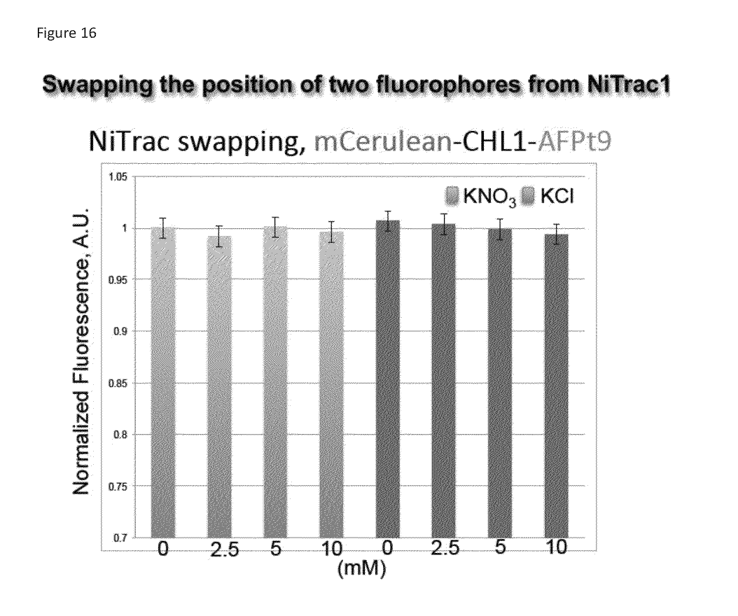

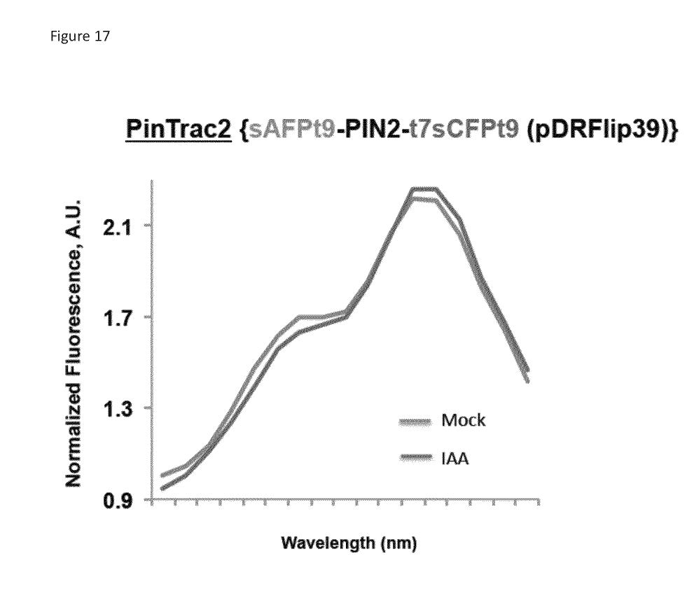

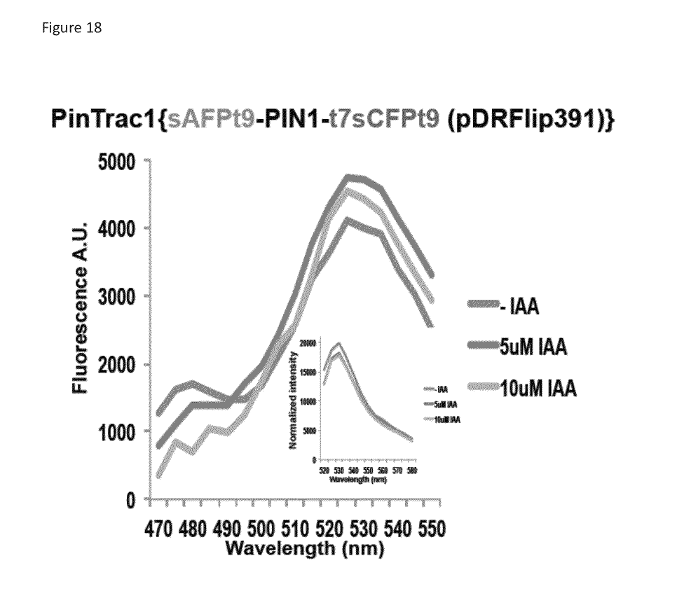

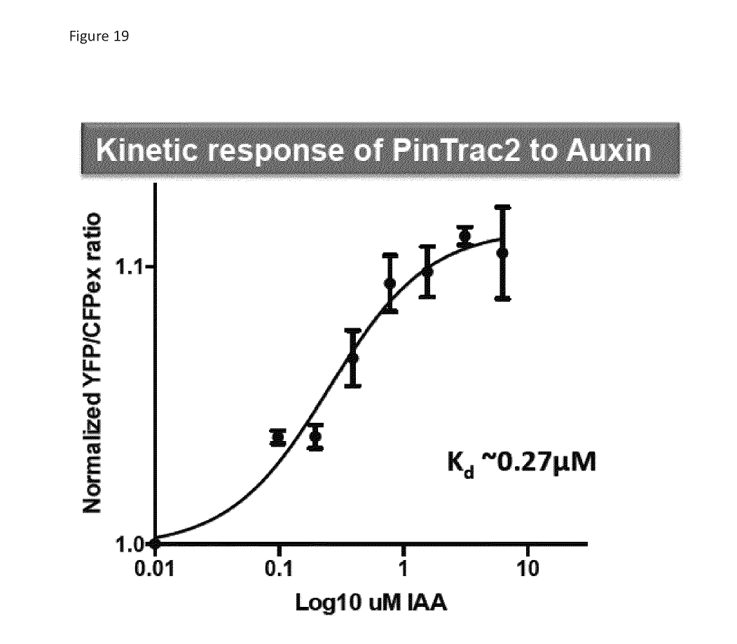

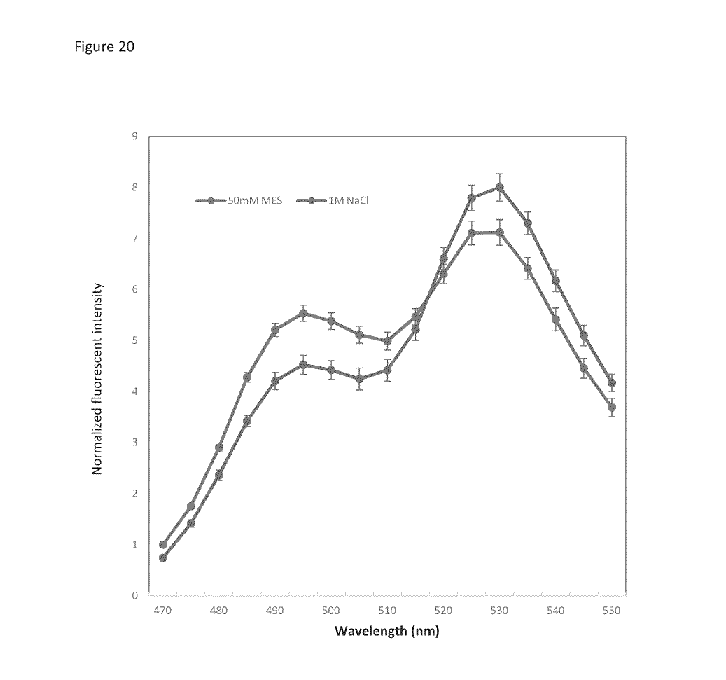

In one embodiment, the nucleic acids of the present invention comprise a polynucleotide sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to polynucleotide sequence in FIG. 1A, (SEQ ID NO:1) (wild-type CHL1 protein of Arabidopsis thaliana). In another embodiment, the nucleic acids of the present invention comprise a polynucleotide sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to polynucleotide sequence in FIG. 1G, (SEQ ID NO:7). In another embodiment, the nucleic acids of the present invention comprise a polynucleotide sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to polynucleotide sequence in FIG. 1H, (SEQ ID NO:8). In another embodiment, the nucleic acids of the present invention comprise a polynucleotide sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to polynucleotide sequence in FIG. 11, (SEQ ID NO:9). In another embodiment, the nucleic acids of the present invention comprise a polynucleotide sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to polynucleotide sequence in FIG. 1J, (SEQ ID NO:10).

In one embodiment, the nucleic acids of the present invention comprise a polynucleotide sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the polynucleotide sequence of SEQ ID NO:12 (cDNA of wild-type PIN1 of Arabidopsis thaliana) or SEQ ID NO: 13 (coding sequence of wild-type PIN1 of Arabidopsis thaliana). In another embodiment, the nucleic acids of the present invention comprise a polynucleotide sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the polynucleotide sequence of SEQ ID NO:15 (cDNA of wild-type PIN2 of Arabidopsis thaliana) or SEQ ID NO: 16 (coding sequence of wild-type PIN2 of Arabidopsis thaliana).

the nucleic acids of the present invention comprise a polynucleotide sequence at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% identical to the polynucleotide sequence of SEQ ID NO:22 (AtMSL10).

The present invention also comprises vectors containing the nucleic acids encoding the fusion proteins of the present invention. As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. An expression vector is one into which a desired DNA sequence may be inserted by restriction and ligation such that it is operably joined to regulatory sequences and may be expressed as an RNA transcript. Vectors may further contain one or more marker sequences suitable for use in the identification and selection of cells, which have been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Examples of vectors include but are not limited to those capable of autonomous replication and expression of the structural gene products present in the DNA segments to which they are operably joined.

In certain respects, the vectors to be used are those for expression of polynucleotides and proteins of the present invention. Generally, such vectors comprise cis-acting control regions effective for expression in a host operatively linked to the polynucleotide to be expressed. Appropriate trans-acting factors are supplied by the host, supplied by a complementing vector or supplied by the vector itself upon introduction into the host.

A great variety of expression vectors can be used to express the proteins of the invention. Such vectors include chromosomal, episomal and virus-derived vectors, e.g., vectors derived from bacterial plasmids, from bacteriophage, from yeast episomes, from yeast chromosomal elements, from viruses such as adeno-associated virus, lentivirus, baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. All may be used for expression in accordance with this aspect of the present invention. Generally, any vector suitable to maintain, propagate or the fusion proteins in a host may be used for expression in this regard.

The DNA sequence in the expression vector is operatively linked to appropriate expression control sequence(s) including, for instance, a promoter to direct mRNA transcription. Representatives of such promoters include, but are not limited to, the phage lambda PL promoter, the E. coli lac, trp and tac promoters, HIV promoters, the SV40 early and late promoters and promoters of retroviral LTRs, to name just a few of the well-known promoters. In general, expression constructs will contain sites for transcription, initiation and termination and, in the transcribed region, a ribosome binding site for translation. The coding portion of the mature transcripts expressed by the constructs will include a translation initiating AUG at the beginning and a termination codon (UAA, UGA or UAG) appropriately positioned at the end of the polypeptide to be translated.

In addition, the constructs may contain control regions that regulate, as well as engender expression. Generally, such regions will operate by controlling transcription, such as repressor binding sites and enhancers, among others.

Vectors for propagation and expression generally will include selectable markers. Such markers also may be suitable for amplification or the vectors may contain additional markers for this purpose. In this regard, the expression vectors may contain one or more selectable marker genes to provide a phenotypic trait for selection of transformed host cells. Preferred markers include dihydrofolate reductase or neomycin resistance for eukaryotic cell culture, and tetracycline, kanamycin or ampicillin resistance genes for culturing E. coli and other bacteria.

Examples of vectors that may be useful for fusion proteins include, but are not limited to, pPZP, pZPuFLIPs, pCAMBIA, and pRT to name a few.

Examples of vectors for expression in yeast S. cerevisiae include pDRFLIP,s, pDR196, pYepSecI (Baldari (1987) EMBO J. 6, 229-234), pMFa (Kurjan (1982) Cell 30, 933-943), pJRY88 (Schultz (1987) Gene 54, 115-123), pYES2 (Invitrogen) and picZ (Invitrogen).

Alternatively, the fusion proteins can be expressed in insect cells using baculovirus expression vectors. Baculovirus vectors available for expression of proteins in cultured insect cells (e.g., SF9 cells) include the pAc series (Smith (1983) Mol. Cell. Biol. 3, 2156 2165) and the pVL series (Lucklow (1989) Virology 170, 31-39).

The nucleic acid molecules of the invention can be “isolated.” As used herein, an “isolated” nucleic acid molecule or nucleotide sequence is intended to mean a nucleic acid molecule or nucleotide sequence that is not flanked by nucleotide sequences normally flanking the gene or nucleotide sequence (as in genomic sequences) and/or has been completely or partially removed from its native environment (e.g., a cell, tissue). For example, nucleic acid molecules that have been removed or purified from cells are considered isolated. In some instances, the isolated material will form part of a composition (for example, a crude extract containing other substances), buffer system or reagent mix. In other circumstances, the material may be purified to near homogeneity, for example as determined by PAGE or column chromatography such as HPLC. Thus, an isolated nucleic acid molecule or nucleotide sequence can includes a nucleic acid molecule or nucleotide sequence which is synthesized chemically, using recombinant DNA technology or using any other suitable method. To be clear, a nucleic acid contained in a vector would be included in the definition of “isolated” as used herein. Also, isolated nucleotide sequences include recombinant nucleic acid molecules (e.g., DNA, RNA) in heterologous organisms, as well as partially or substantially purified nucleic acids in solution. “Purified,” on the other hand is well understood in the art and generally means that the nucleic acid molecules are substantially free of cellular material, cellular components, chemical precursors or other chemicals beyond, perhaps, buffer or solvent. “Substantially free” is not intended to mean that other components beyond the novel nucleic acid molecules are undetectable. The nucleic acid molecules of the present invention may be isolated or purified. Both in vivo and in vitro RNA transcripts of a DNA molecule of the present invention are also encompassed by “isolated” nucleotide sequences.

The invention also provides nucleic acid molecules that hybridize under high stringency hybridization conditions, such as for selective hybridization, to the nucleotide sequences described herein (e.g., nucleic acid molecules which specifically hybridize to a nucleotide sequence encoding fusion proteins described herein and encode a transporter protein and/or one or more fluorescent proteins). Hybridization probes include synthetic oligonucleotides which bind in a base-specific manner to a complementary strand of nucleic acid.

Such nucleic acid molecules can be detected and/or isolated by specific hybridization, e.g., under high stringency conditions. “Stringency conditions” for hybridization is a term of art that refers to the incubation and wash conditions, e.g., conditions of temperature and buffer concentration, which permit hybridization of a particular nucleic acid to a second nucleic acid; the first nucleic acid may be perfectly complementary, i.e., 100%, to the second, or the first and second may share some degree of complementarity, which is less than perfect, e.g., 60%, 75%, 85%, 95% or more. For example, certain high stringency conditions can be used which distinguish perfectly complementary nucleic acids from those of less complementarity.

“High stringency conditions”, “moderate stringency conditions” and “low stringency conditions” for nucleic acid hybridizations are explained in Current Protocols in Molecular Biology, John Wiley & Sons). The exact conditions which determine the stringency of hybridization depend not only on ionic strength, e.g., 0.2×SSC, 0.1×SSC of the wash buffers, temperature, e.g., room temperature, 42° C., 68° C., etc., and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, high, moderate or low stringency conditions may be determined empirically.

By varying hybridization conditions from a level of stringency at which no hybridization occurs to a level at which hybridization is first observed, conditions which will allow a given sequence to hybridize with the most similar sequences in the sample can be determined. Exemplary conditions are described in Krause (1991) Methods in Enzymology, 200:546-556. Washing is the step in which conditions are usually set so as to determine a minimum level of complementarity of the hybrids. Generally, starting from the lowest temperature at which only homologous hybridization occurs, each degree (° C.) by which the final wash temperature is reduced, while holding SSC concentration constant, allows an increase by 1% in the maximum extent of mismatching among the sequences that hybridize. Generally, doubling the concentration of SSC results in an increase in Tm. Using these guidelines, the washing temperature can be determined empirically for high, moderate or low stringency, depending on the level of mismatch sought. Exemplary high stringency conditions include, but are not limited to, hybridization in 50% formamide, 1 M NaCl, 1% SDS at 37° C., and a wash in 0.1×SSC at 60° C. Examples of progressively higher stringency conditions include, after hybridization, washing with 0.2×SSC and 0.1% SDS at about room temperature (low stringency conditions), washing with 0.2×SSC, and 0.1% SDS at about 42° C. (moderate stringency conditions), and washing with 0.1×SSC at about 68° C. (high stringency conditions). Washing can be carried out using only one of these conditions, e.g., high stringency conditions, washing may encompass two or more of the stringency conditions in order of increasing stringency. Optimal conditions will vary, depending on the particular hybridization reaction involved, and can be determined empirically.

Equivalent conditions can be determined by varying one or more of the parameters given as an example, as known in the art, while maintaining a similar degree of identity or similarity between the target nucleic acid molecule and the primer or probe used. Hybridizable nucleotide sequences are useful as probes and primers for identification of organisms comprising a nucleic acid of the invention and/or to isolate a nucleic acid of the invention, for example. The term “primer” is used herein as it is in the art and refers to a single-stranded oligonucleotide, which acts as a point of initiation of template-directed DNA synthesis under appropriate conditions in an appropriate buffer and at a suitable temperature. The appropriate length of a primer depends on the intended use of the primer, but typically ranges from about 15 to about 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

The present invention also relates to host cells containing the above-described constructs. The host cell can be a eukaryotic cell, such as a plant cell or yeast cell, or the host cell can be a prokaryotic cell, such as a bacterial cell. The host cell can be stably or transiently transfected with the construct. The polynucleotides may be introduced alone or with other polynucleotides. Such other polynucleotides may be introduced independently, co-introduced or introduced joined to the polynucleotides of the invention. As used herein, a “host cell” is a cell that normally does not contain any of the nucleotides of the present invention and contains at least one copy of the nucleotides of the present invention. Thus, a host cell as used herein can be a cell in a culture setting or the host cell can be in an organism setting where the host cell is part of an organism, organ or tissue.

If a prokaryotic expression vector is employed, then the appropriate host cell would be any prokaryotic cell capable of expressing the cloned sequences. Suitable prokaryotic cells include, but are not limited to, bacteria of the genera Escherichia, Bacillus, Pseudomonas, Staphylococcus, and Streptomyces.

If a eukaryotic expression vector is employed, then the appropriate host cell would be any eukaryotic cell capable of expressing the cloned sequence. In one embodiment, eukaryotic cells are the host cells. Eukaryotic host cells include, but are not limited to, insect cells, HeLa cells, Chinese hamster ovary cells (CHO cells), African green monkey kidney cells (COS cells), human 293 cells, and murine 3T3 fibroblasts.

In addition, a yeast cell may be employed as a host cell. Yeast cells include, but are not limited to, the genera Saccharomyces, Pichia and Kluveromyces. In one embodiment, the yeast hosts are S. cerevisiae or P. pastoris. Yeast vectors may contain an origin of replication sequence from a 2T yeast plasmid, an autonomously replication sequence (ARS), a promoter region, sequences for polyadenylation, sequences for transcription termination and a selectable marker gene. Shuttle vectors for replication in both yeast and E. coli are also included herein.

Introduction of a construct into the host cell can be affected by calcium phosphate transfection, DEAE-dextran mediated transfection, cationic lipid-mediated transfection, electroporation, transduction, infection or other methods.

Other examples of methods of introducing nucleic acids into host organisms take advantage TALEN technology to effectuate site-specific insertion of nucleic actions. TALENs are proteins that have been engineered to cleave nucleic acids at a specific site in the sequence. The cleavage sites of TALENs are extremely customizable and pairs of TALENs can be generated to create double-stranded breaks (DSBs) in nucleic acids at virtually any site in the nucleic acid. See Bogdanove and Voytas, Scienc, 333:1843-1846 (2011), which incorporated by reference herein

Transformants carrying the expression vectors are selected based on the above-mentioned selectable markers. Repeated clonal selection of the transformants using the selectable markers allows selection of stable cell lines expressing the fusion proteins constructs. Increasing the concentration in the selection medium allows gene amplification and greater expression of the desired fusion proteins. The host cells, for example E. coli cells, containing the recombinant fusion proteins can be produced by cultivating the cells containing the fusion proteins expression vectors constitutively expressing the fusion proteins constructs.

The present invention also provides for transgenic plants or plant tissue comprising transgenic plant cells, i.e. comprising stably integrated into their genome, an above-described nucleic acid molecule, expression cassette or vector of the invention. The present invention also provides transgenic plants, plant cells or plant tissue obtainable by a method for their production as outlined below.

In one embodiment, the present invention provides a method for producing transgenic plants, plant tissue or plant cells comprising the introduction of a nucleic acid molecule, expression cassette or vector of the invention into a plant cell and, optionally, regenerating a transgenic plant or plant tissue therefrom. The transgenic plants expressing the fusion protein can be of use in monitoring the transport or movement of nitrate, peptide or hormones throughout and between the organs of an organism, such as to or from the soil. The transgenic plants expressing transporters of the invention can be of use for investigating metabolic or transport processes of, e.g., organic compounds with a timely and spatial resolution.

Examples of species of plants that may be used for generating transgenic plants include but are not limited to monocotyledonous plants including seed and the progeny or propagules thereof, for example Lolium, Zea, Triticum, Sorghum, Triticale, Saccharum, Bromus, Oryzae, Avena, Hordeum, Secale and Setaria. Especially useful transgenic plants are maize, wheat, barley plants and seed thereof. Dicotyledenous plants are also within the scope of the present invention include but are not limited to the species Fabaceae, Solanum, Brassicaceae, especially potatoes, beans, cabbages, forest trees, roses, clematis, oilseed rape, sunflower, chrysanthemum, poinsettia and antirrhinum (snapdragon). The plant may be crops, such as a food crops, feed crops or biofuels crops. Exemplary important crops may include soybean, cotton, rice, millet, sorghum, sugarcane, sugar beet, tomato, grapevine, citrus (orange, lemon, grapefruit, etc), lettuce, alfalfa, fava bean and strawberries, rapeseed, cassava, miscanthus and switchgrass to name a few.

Methods for the introduction of foreign nucleic acid molecules into plants are well-known in the art. For example, plant transformation may be carried out using Agrobacterium-mediated gene transfer, microinjection, electroporation or biolistic methods as it is, e.g., described in Potrykus and Spangenberg (Eds.), Gene Transfer to Plants. Springer Verlag, Berlin, New York, 1995. Therein, and in numerous other references, useful plant transformation vectors, selection methods for transformed cells and tissue as well as regeneration techniques are described which are known to the person skilled in the art and may be applied for the purposes of the present invention.

In another aspect, the invention provides harvestable parts and methods to propagation material of the transgenic plants according to the invention, which contain transgenic plant cells as described above. Harvestable parts can be in principle any useful part of a plant, for example, leaves, stems, fruit, seeds, roots etc. Propagation material includes, for example, seeds, fruits, cuttings, seedlings, tubers, rootstocks etc.

The present invention also provides methods of producing any of the fusion proteins of the present invention. In some embodiments, the methods comprise culturing a host cell in conditions that promote protein expression and recovering the fusion protein from the culture, wherein the host cell comprises a vector encoding a fusion protein, wherein the fusion protein comprises at least one fluorescent protein, and at least one transporter protein comprising an N-terminus and a C-terminus, wherein the transporter changes three-dimensional conformation upon specifically transporting its substrate, and at least one fluorescent protein linker peptide, wherein the at least one fluorescent protein linker peptide links the at least one fluorescent protein to the N-terminus or C-terminus of the at least one transporter protein. The methods also comprise culturing a host cell in conditions that promote protein expression and recovering the fusion protein from the culture, wherein the host cell comprises a vector encoding a fusion protein, wherein the fusion protein comprises at least a first and second fluorescent protein, wherein the first and second fluorescent proteins emit wavelengths of light that are different from one another and at least one transporter protein comprising an N-terminus and a C-terminus, wherein the transporter protein changes three-dimensional conformation upon specifically transporting its substrate, and at least a first and second fluorescent protein linker peptide, wherein the first fluorescent protein linker peptide links the first fluorescent protein to the N-terminus of the at least one transporter protein and the second fluorescent protein linker peptide links the second fluorescent protein to the C-terminus of the at least one transporter protein.

The present invention also provides methods of producing any of the fusion proteins of the present invention. In some embodiments, the methods comprise culturing a host cell in conditions that promote protein expression and recovering the fusion protein from the culture, wherein the host cell comprises a vector encoding a fusion protein, wherein the fusion protein comprises at least one fluorescent protein, and at least one mechanosensitive ion channel protein comprising an N-terminus and a C-terminus, and at least one fluorescent protein linker peptide, wherein the at least one fluorescent protein linker peptide links the at least one fluorescent protein to the N-terminus or C-terminus of the at least one mechanosensitive ion channel protein. The methods also comprise culturing a host cell in conditions that promote protein expression and recovering the fusion protein from the culture, wherein the host cell comprises a vector encoding a fusion protein, wherein the fusion protein comprises at least a first and second fluorescent protein, wherein the first and second fluorescent proteins emit wavelengths of light that are different from one another and at least one mechanosensitive ion channel protein comprising an N-terminus and a C-terminus, and at least a first and second fluorescent protein linker peptide, wherein the first fluorescent protein linker peptide links the first fluorescent protein to the N-terminus of the at least one mechanosensitive ion channel protein and the second fluorescent protein linker peptide links the second fluorescent protein to the C-terminus of the at least one mechanosensitive ion channel protein.

The protein production methods generally comprise culturing the host cells of the invention under conditions such that the fusion protein is expressed, and recovering said protein. The culture conditions required to express the proteins of the current invention are dependent upon the host cells that are harboring the polynucleotides of the current invention. The culture conditions for each cell type are well-known in the art and can be easily optimized, if necessary. For example, a nucleic acid encoding a fusion protein of the invention, or a construct comprising such nucleic acid, can be introduced into a suitable host cell by a method appropriate to the host cell selected, e.g., transformation, transfection, electroporation, infection, such that the nucleic acid is operably linked to one or more expression control elements as described herein. Host cells can be maintained under conditions suitable for expression in vitro or in vivo, whereby the encoded fusion protein is produced. For example host cells may be maintained in the presence of an inducer, suitable media supplemented with appropriate salts, growth factors, antibiotic, nutritional supplements, etc., which may facilitate protein expression. In additional embodiments, the fusion proteins of the invention can be produced by in vitro translation of a nucleic acid that encodes the fusion protein, by chemical synthesis or by any other suitable method. If desired, the fusion protein can be isolated from the host cell or other environment in which the protein is produced or secreted. It should therefore be appreciated that the methods of producing the fusion proteins encompass expression of the polypeptides in a host cell of a transgenic plant. See U.S. Pat. Nos. 6,013,857, 5,990385, and 5,994,616.

The invention also provides for methods of measuring and/or monitoring nitrate, peptide or hormone levels in a sample, comprising contacting the sample with a fusion protein of the present invention and subsequently measuring the change in luminescence that occurs in response to the presence or absence of the substrate.

The invention also provides for methods of measuring mechanosensitive ion channel protein activities in the sample, comprising monitoring the sample with a fusion protein of the present invention and subsequently measuring the change in luminescence that occurs in response to mechanical signal and/or osmetic stress.

Changes in luminesence can mean any detectable change in a property of the at least one fluorophore. For example, a change in luminescence includes but is not limited to a change of the wavelength, intensity, lifetime, energy transfer efficiency, and/or polarization of the fluorophore. In one embodiment, the change in luminescence is FRET-based. In another embodiment, the change in luminescence is not FRET-based. For example, in non-FRET-based changes in luminescence, the one or more of the fluorescent proteins of the fusion constructs may exhibit an increase or decrease in emission intensity in response to substrate transport or possible binding. Other detectable changes in the properties of the fluorophores that may or may not be FRET-based include but are not limited to shift in emission wavelength, intensity, lifetime, energy transfer efficiency, and/or polarization of the luminescence of the at least one of the fluorescent reporters.

Accordingly, the fusion proteins can be used in sensors for measuring or monitoring nitrates or peptides (substrates) in a sample, with the sensors comprising the fusion proteins of the present invention.

The fusion proteins of the current invention can be used to assess, measure or monitor the concentrations of nitrate, peptide or hormone substrates. As used herein, concentration is used as it is in the art. The concentration may be expressed as a qualitative value, or more likely as a quantitative value. As used herein, the quantification of substrate can be a relative or absolute quantity. Of course, the quantity (concentration) of any substrate may be equal to zero, indicating the absence of substrate. The quantity may simply be the measured signal, e.g., fluorescence, without any additional measurements or manipulations. Alternatively, the quantity may be expressed as a difference, percentage or ratio of the measured value of the particular analyte to a measured value of another compound including, but not limited to, a standard. The difference may be negative, indicating a decrease in the amount of measured nitrate. The quantities may also be expressed as a difference or ratio of the substrate to itself, measured at a different point in time. The quantities of substrate may be determined directly from a generated signal, or the generated signal may be used in an algorithm, with the algorithm designed to correlate the value of the generated signals to the quantity of substrate(s) in the sample.

In some embodiments, the fusion proteins of the current invention are designed to possess capabilities of continuously measuring the concentration of substrates. As used herein, the term “continuously,” in conjunction with the measuring of a substrate, is used to mean the fusion protein either generates or is capable of generating a detectable signal at any time during the life span of the fusion protein. The detectable signal may be constant in that the fusion protein is always generating a signal, even if the signal is not detected. Alternatively, the fusion protein may be used episodically, such that a detectable signal may be generated, and detected, at any desired time.

In one embodiment, the substrate being measured or monitored is not labeled. While not a requirement of the present invention, the fusion proteins are particularly useful in an in vivo setting for measuring or monitoring substrates as they occur or appear in a plant or plant tissue. As such, the target substrates need not be labeled. Of course, unlabeled substrates may also be measured in an in vitro or in situ setting as well. In another embodiment, the substrate(s) may be labeled. Labeled target substrates can be measured in an in vivo, in vitro or in situ setting.

Examples of nitrate containing compounds include but are not limited to acids containing nitrate, e.g., nitric acid (HNO₃), peroxynitric acid (HNO₄), and esters of nitric acid, organic and inorganic salts containing nitrate. Examples of salts containing nitrates include but are not limited to sodium nitrate and potassium nitrate. Other nitrate containing compounds include but are not limited to ammonium nitrate (NH₄NO₃).

Examples of peptides as substrates include but are not limited to di-peptides, tri-peptides and longer peptide chains. The peptide substrates are known for each specific peptide transporter. For example, substrates for the hPEPT1 and hPEPT2 transporters include those substrates listed in Table 1 of Rubio-Aliaga, I. and Daniel, H., Xenobiotica, 38(7-8):1022-1042 (2008), which has already been incorporated by reference in its entirety.

Purified biosensor can also be incorporated into kits for measurement or monitoring of substrates in various samples. The samples would require minimal processing, thus the kit would allow high-throughput substrate measurement or monitoring in complex samples using an appropriate plate fluorometer (e.g. TECAN M1000). This type of analysis can be used to measure the substrate content in different tissues, different individual plants or different populations of, for example, crop plants experiencing drought or crop plants in poor soil conditions. Purification of bulk amounts of biosensor can be achieved after expression in Pichia pastoris, using pPinkFLIP vectors and a protease deficient strain of Pichia.

The inventors developed a novel and generalizable platform for systematic conversion of transporters and channels. Fusion proteins comprising nitrate transporter were developed, demonstrating that fusion to fluorescent proteins can be used to monitor transporter proteins activity. This approach is generalizable by one step creation of multiple peptide transport activity sensors. These sensors all report activity as a change of fluorescence—either loss of absorption or quenching of one fluorophore, both fluorophores or a FRET change. These transporter proteins belong to the Major Facilitator Superfamily and the efficient conversion demonstrates that any MFS transporter can be converted into a sensor using this approach. Only modifications in the linkers may be necessary to adjust the position in order to obtain a high sensitivity activity sensor.

To further demonstrate the broad applicability, the inventors used a different scaffold—a PIN auxin transporter. Importantly, in contrast to the nitrate and peptide importers, PINs are exporters. Activity sensors were developed based on the PIN transporters, although these proteins are very different and unrelated to the MFS superfamily.

The inventors also used another different scaffold—a protein that acts as an ion channel, and in particular a mechanosensitive ion channel protein. The fusion protein may be used to measure the membrane tension dependent activity of the MSL channel. This channel is structurally different (Veley et al., Plant Cell. 2014; 26(7):3115-31) from the nitrate transporters and hormone transporter. Importantly, this sensor can not only be used to track the activity of the channel, but also measure physical phenomena, i.e. cell turgor, as a proxy of membrane tension.

By presenting a number of constructs from different molecular families, it is thus unambiguously shown that the approach described is generalizable.

The examples herein are provided for illustrative purposed and are not intended to limit the scope of the invention in any way.

### EXAMPLES

### Example 1

### Nitrate Sensor

All transporter and sensor constructs were inserted in the yeast expression vector pDRFlip30, 34, 35, 39, 42-GW. The details of the vectors are as follows: pDRFlip30 using pair of N-terminal fluorescent protein Aphordite t9 (AFPt9), 9 amino acids truncated of C-term of AFP, and C-terminal fluorescent protein monomeric Cerulean (mCer); pDRFlip 39 using pair of N-terminally fused fluorescent protein enhanced dimer Aphrodite t9 (edAFPt9) and C-terminal fluorescent protein enhanced dimer, 7 amino acids and 9 amino acids truncated of N-term and C-term of eCyan (t7.ed.eCFPt9), respectively; pDRFlip 42 using pair of N-terminal fluorescent protein Citrine and C-terminal fluorescent protein mCer; pDRFlip 34 using pair of N-terminal fluorescent protein AFPt9 and C-terminal fluorescent protein t7.Teal.t9 (t7.TFP.t9), and pDRFlip 35 using pair of N-terminal fluorescent protein AFPt9 and C-terminal fluorescent protein mTFPt9. All vectors contained theft replication origin, GATEWAY™ cassette-attR1-CmR-ccdB gene-attR2 sequence, which is between the pair of fluorescent proteins, a PMA1 promoter fragment, an ADH terminator, different pairs of fluorescent proteins, and the URA cassette for selection in yeast. The full length ORF of NRT1.1 and different mutants of NRT1.1, such as T101A, T101D, P492L from Arabidopsis (At1g12110) in TOPO GATEWAY™ entry vector were used to prepare the nitrate sensors of the present invention. The yeast vector harboring the constructs was then created by the GATEWAY™ LR reaction between different forms of pTOPO-NRT and different pDRFlip-GWs, following manufacturer's instructions..

### Example 2

### Testing of Nitrate Sensors

Yeast strains used in this study were BJ5465 [MATa, ura3-52, trp1, leu2Δ1, his3Δ200, pep4::HIS3, prb1Δ1.6R, cant GAL+] obtained from Yeast Genetic Stock Center (University of California, Berkeley, Calif.). Yeast was transformed using the lithium acetate method and selected on solid YNB (minimal yeast medium without nitrogen; Difco) supplemented with 2% glucose and -Ura DropOut (Clontech). Single colonies were grown in 5 mL liquid YNB supplemented with 2% glucose and -Ura DropOut under agitation (220 rpm) at 30° C. until OD_{600 nm}˜0.8 was reached. The liquid cultures were subcultured by diluted to OD_{600 nm} 0.01 in the same liquid medium and conditions at 30° C. until OD_{600 nm} 0.2 was reached. Yeast cultures were then washed twice in 50 mM MES buffer, pH 5.5, and resuspended to OD_{600 nm}˜0.5 in the same MES buffer supplemented with 0.05% agarose to delay cell sedimentation. Fluorescence was measured by a fluorescence plate reader (M1000, TECAN), in bottom reading mode using a 7.5 nm bandwidth for both excitation and emission. To measure fluorescence response to substrate addition, 100 μL of substrate (dissolved in MES buffer as 500% stock solution) were added to 100 μL of cells in a 96-well plate (Greiner). Fluorescence from cultures harboring yeast expression vectors pDRFlip30, 39, and 42 was measured as emission at λₑₘ=470-570 nm using excitation at λ_{exc}=428 nm and fluorescence using yeast expression vector pDRFlip34 and 35 was measured as emission at λₑₘ=470-570 nm using excitation at λ_{exc}=440 nm.

### Example 3

### Peptide Sensor

All transporter and sensor constructs were inserted in the yeast expression vector pDRFlip30, 34, 35, 39, 42-GW, containing the f1 replication origin, GATEWAY™ cassette, a PMA1 promoter fragment, an ADH terminator, different pairs of fluorescent proteins, and the URA cassette for selection in yeast. The full length ORF of PTR1, 2, 4, and 5 from Arabidopsis (At3g54140, At2g02040, At2g02020, and At5g01180, respectively) in the TOPO GATEWAY™ entry vector were used to create the peptide sensors. The yeast expression vector harboring the constructs was then created by the GATEWAY™ LR reaction between different forms of pTOPO-NRT or pTOPO-PTR and different pDRFlip-GWs, following manufacturer's instructions.

### Example 4

### Testing of Peptide Sensor

Yeast strains used in this study were BJ5465 [MATa, ura3-52, trp1, leu2Δ1, his3 Δ200, pep4::HIS3, prb1Δ1.6R, cant GAL+] obtained from Yeast Genetic Stock Center (University of California, Berkeley, Calif.). Yeast was transformed using the lithium acetate method and selected on solid YNB (minimal yeast medium without nitrogen; Difco) supplemented with 2% glucose and -Ura DropOut (Clontech). Single colonies were grown in 5 mL liquid YNB supplemented with 2% glucose and -Ura DropOut under agitation (220 rpm) at 30° C. until OD_{600 nm}˜0.5 was reached. The liquid cultures were subcultured by diluted to OD_{600 nm} 0.01 in the same liquid medium and conditions at 30° C. until OD_{600 nm}˜0.2 was reached. Yeast cultures were then washed twice in 50 mM MES buffer, pH 5.5, and resuspended to OD_{600 nm}˜0.5 in the same MES buffer supplemented with 0.05% agarose to delay cell sedimentation. Fluorescence was measured by a fluorescence plate reader (M1000, TECAN), in bottom reading mode using a 7.5 nm bandwidth for both excitation and emission. To measure fluorescence response to substrate addition, 100 μL of substrate (dissolved in MES buffer as 500% stock solution) were added to 100 μL of cells in a 96-well plate (Greiner). Fluorescence from cultures containing the yeast expression vector pDRFlip30, 39, or 42 was measured as emission at λₑₘ=470-570 nm using excitation at λ_{exc}=428 nm and fluorescence from cultures containing the yeast expression vectors pDRFlip34 or 35 was measured as emission at λₑₘ=470-570 nm using excitation at λ_{exc}=440 nm.

### Example 5

### Testing of Osmosensors

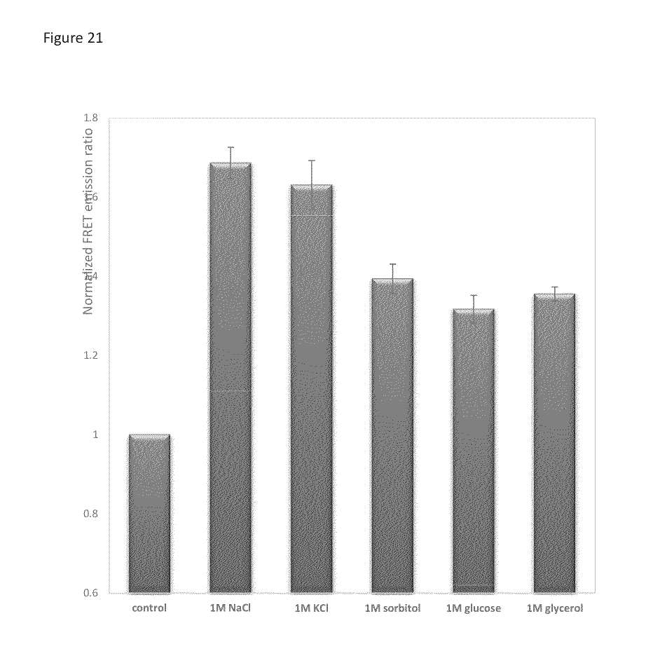

Fusion proteins comprising the mechanisensitive channel small conductance-like 10 (AtMSL10) were constructed, potentially creating an osmosensor. Among these, a fusion protein comprising AtMSL10, a truncated Aphrodite (t9AFP), and a truncated TFP (t7TFPt9) flourophore showed dramatic FRET change response to 1M sodium chloride (NaCl) treatment. See FIGS. 20-22. This t9AFP-AtMSL10-t7TFPt9 protein is named as OzTrac-MSL10. When OzTrac-MSL10 was expressed in yeast cells, it showed correct localization to the plasma membrane, but it also accumulated in endomembranes. Upon treatment of 1 M NaCl, which induces hyper-osmotic stress, AtMSL10 will undergo a conformational change into the closed state which causes the FRET pairs to come closer, resulting in a higher FRET. See FIG. 20. In order to show that the FRET response is due to changes in osmotic pressure and not from the sodium chloride itself, other osmolytes including potassium chloride, sorbitol, glucose and glycerol, the addition of which also increased the FRET, indicating that OzTrac-MSL10 is a sensor that is sensitive to osmotic stress. See FIG. 21.

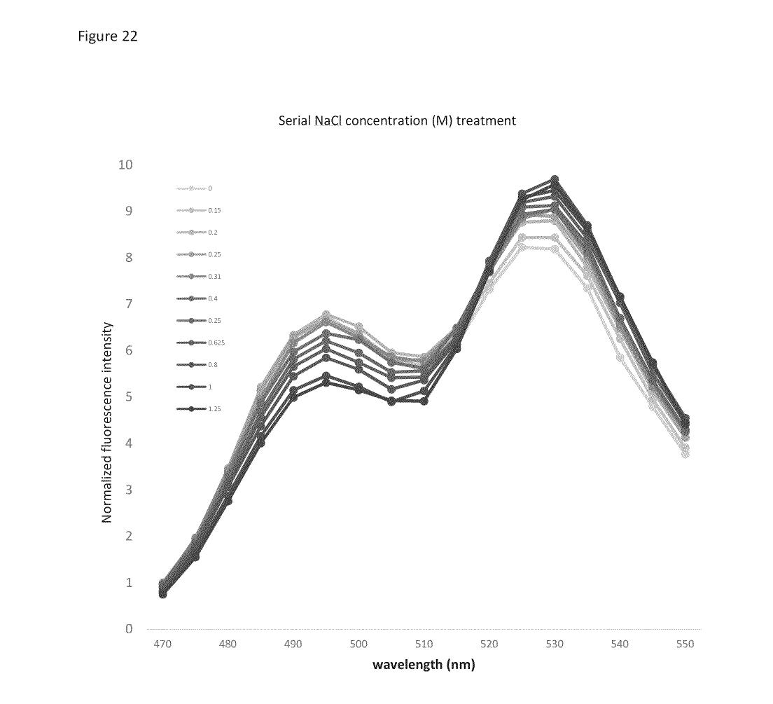

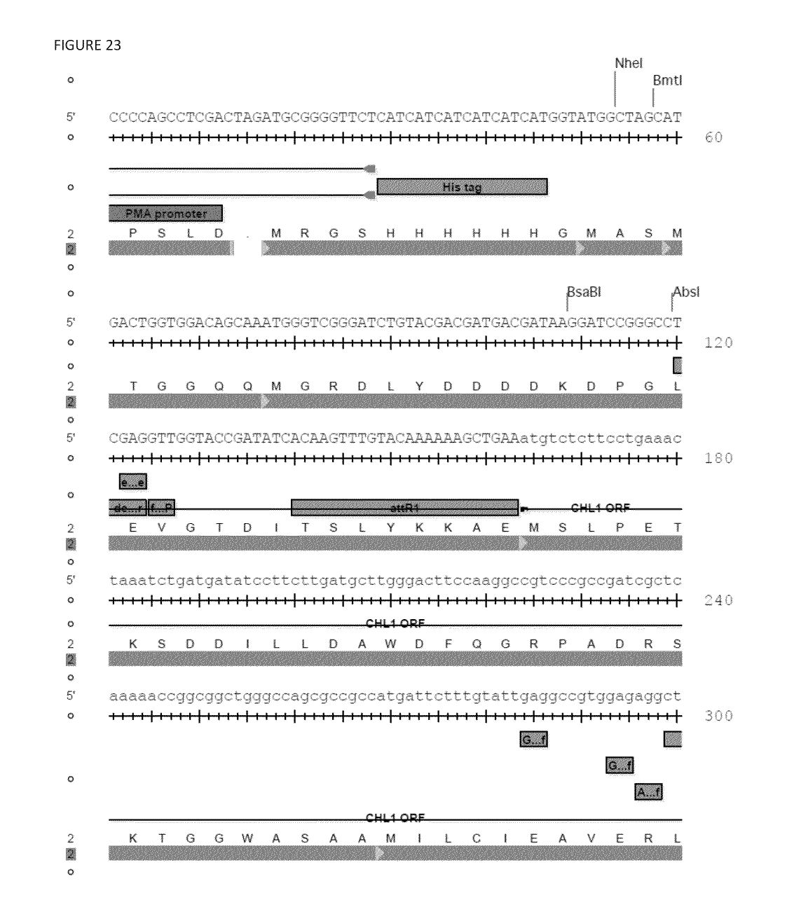

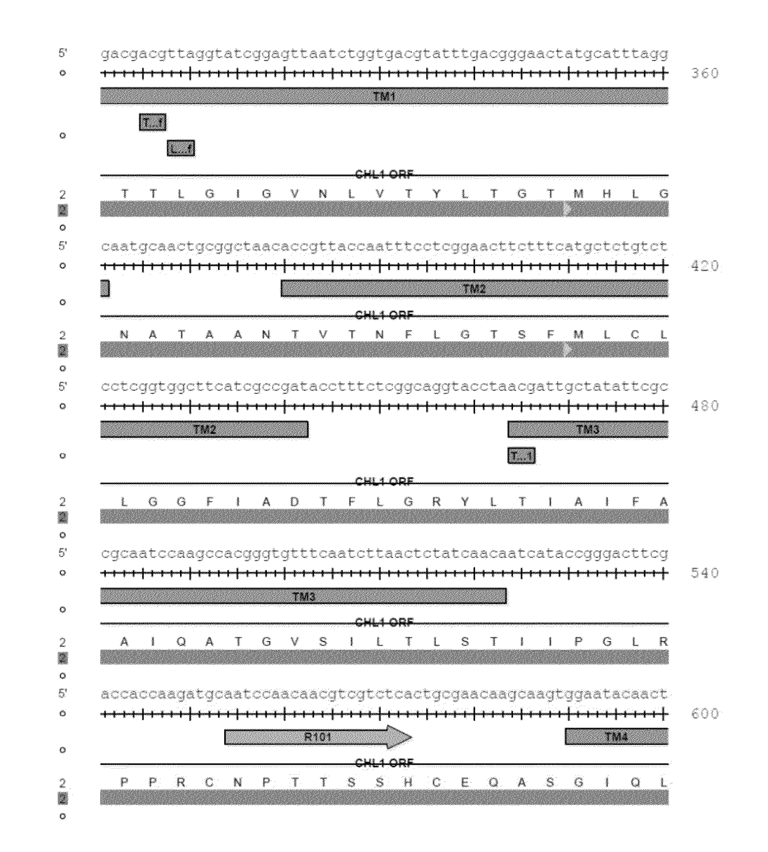

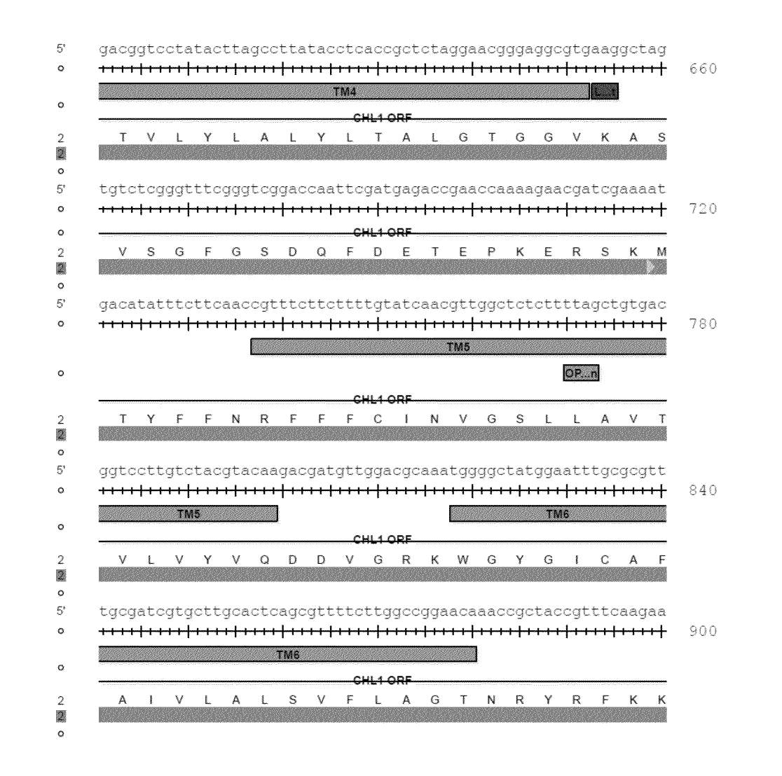

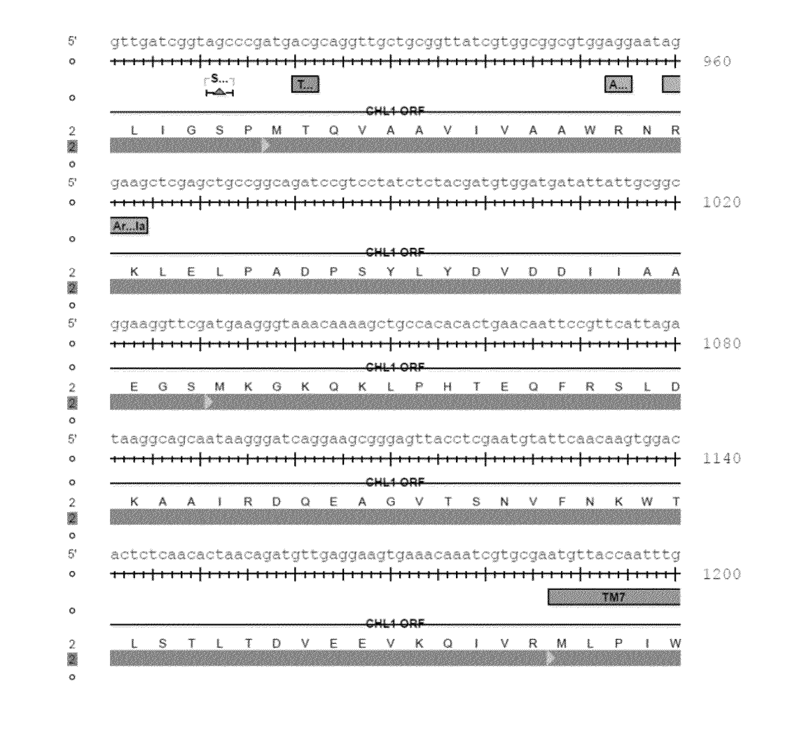

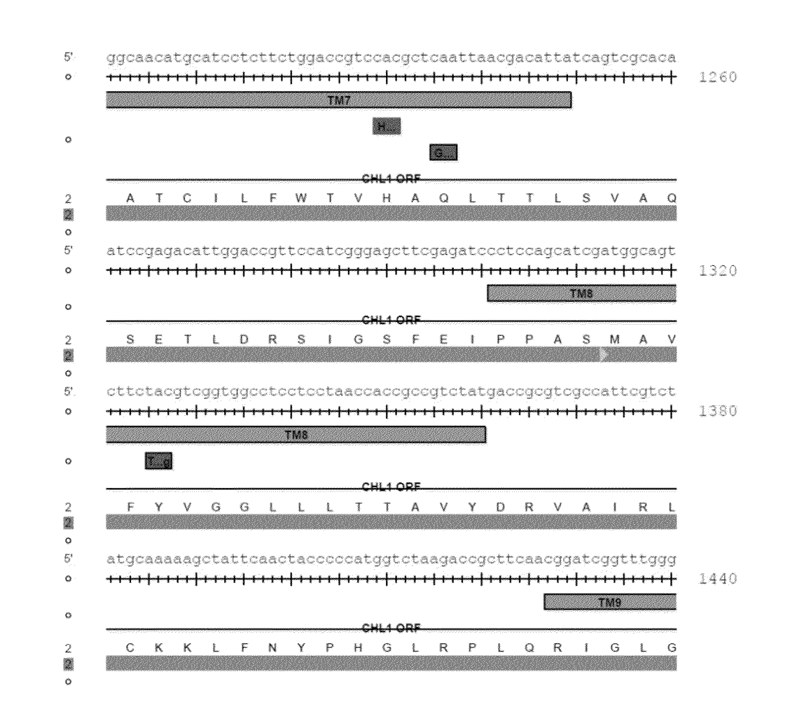

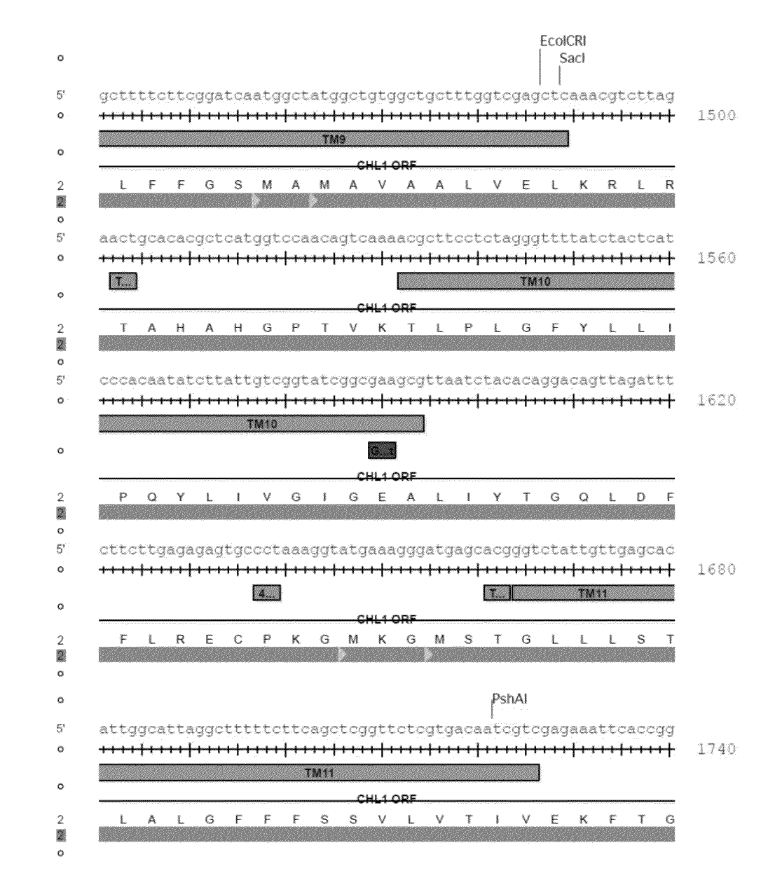

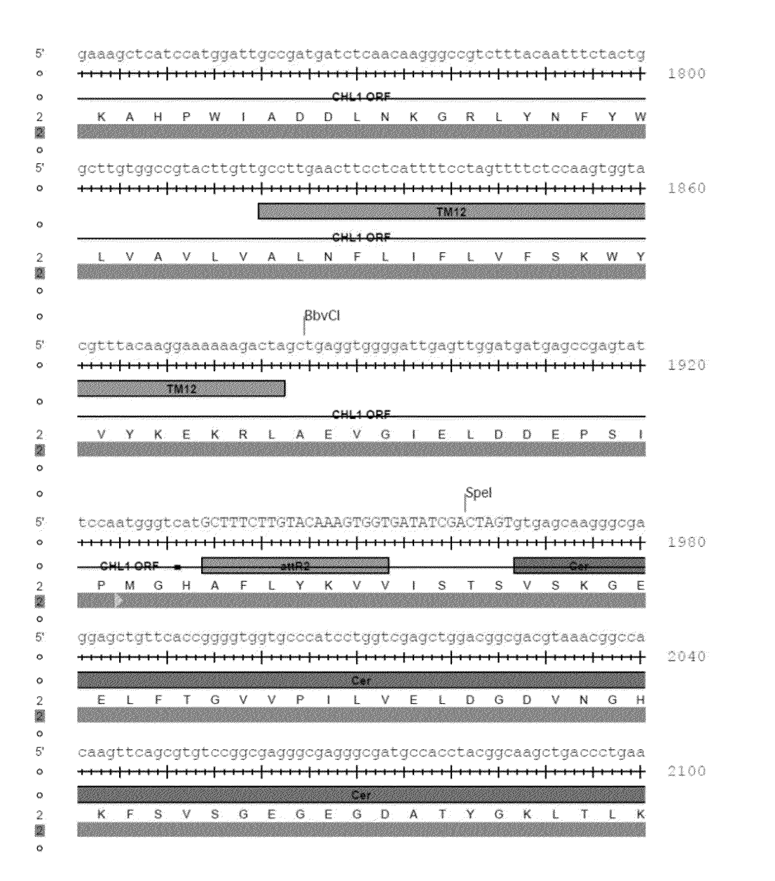

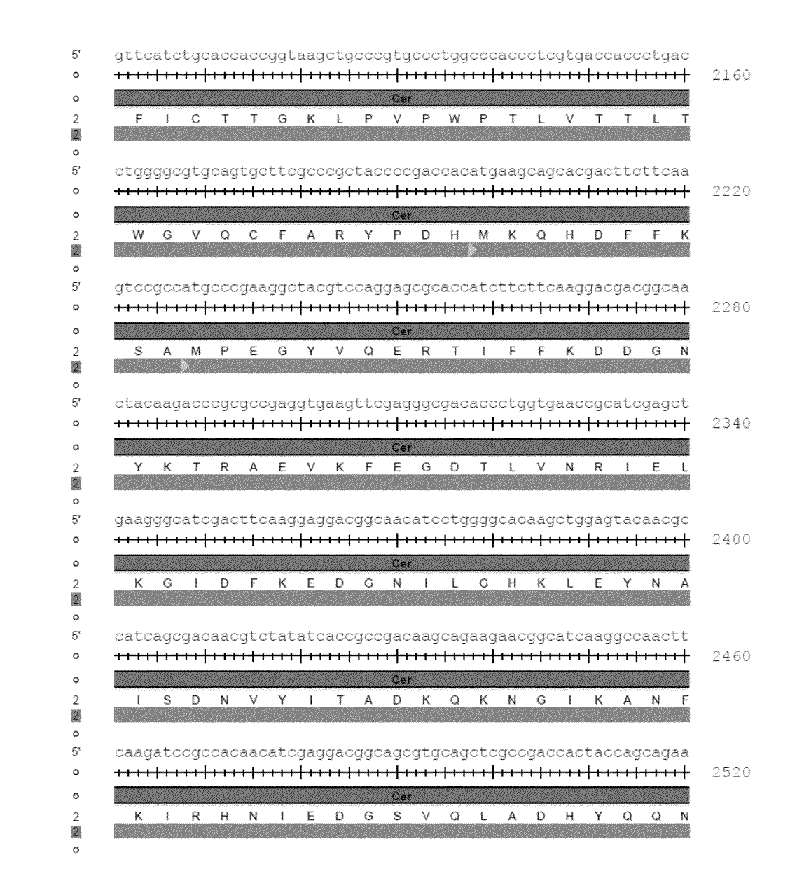

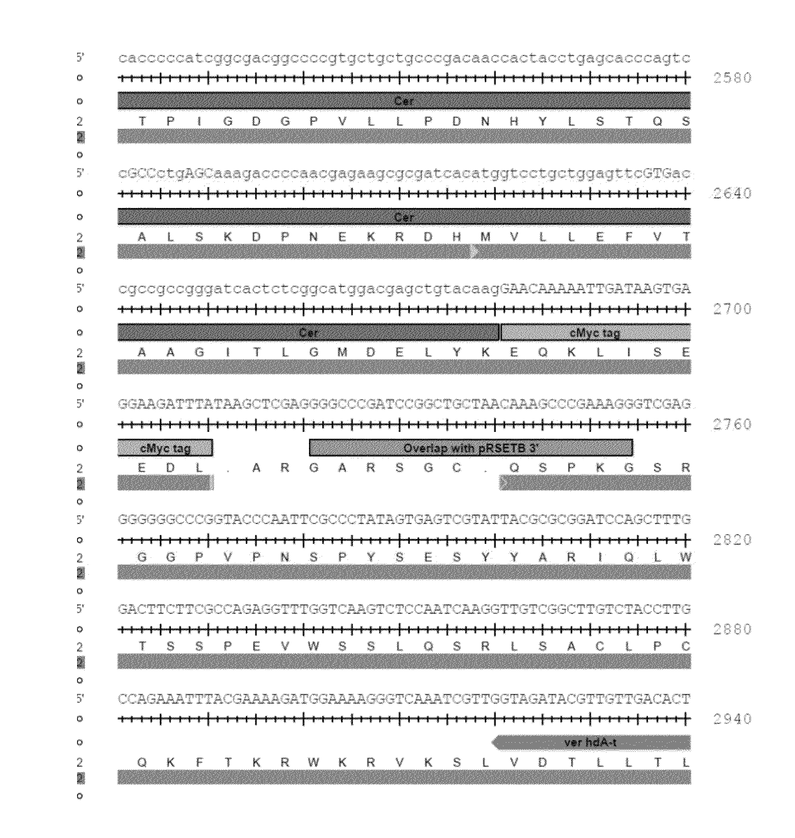

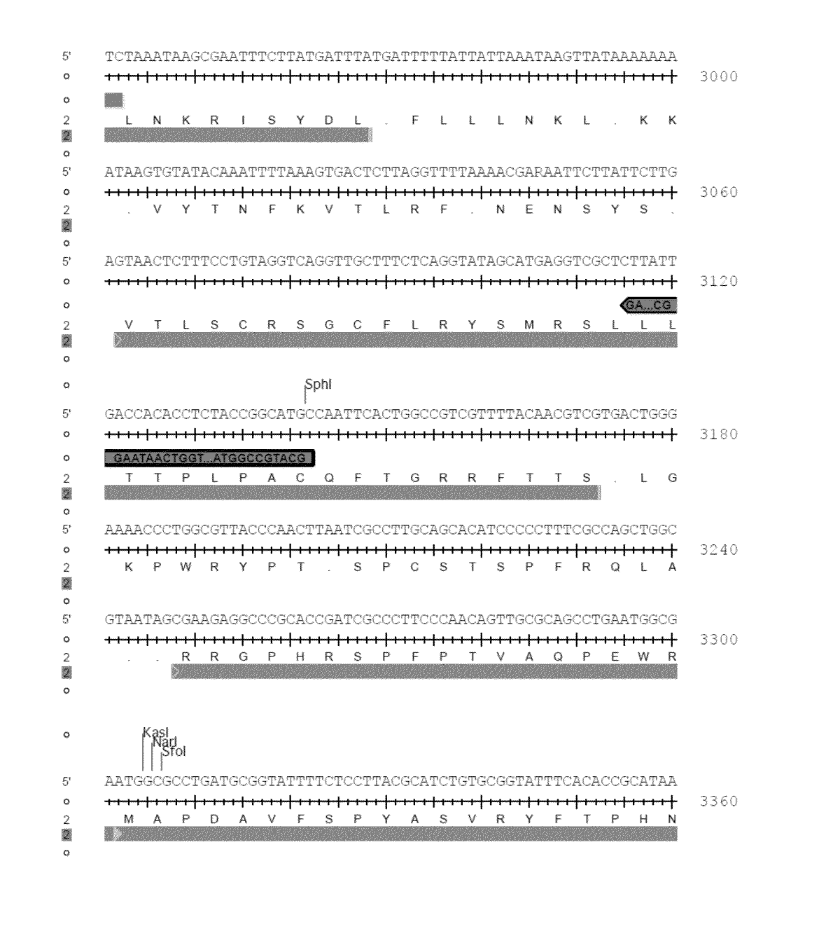

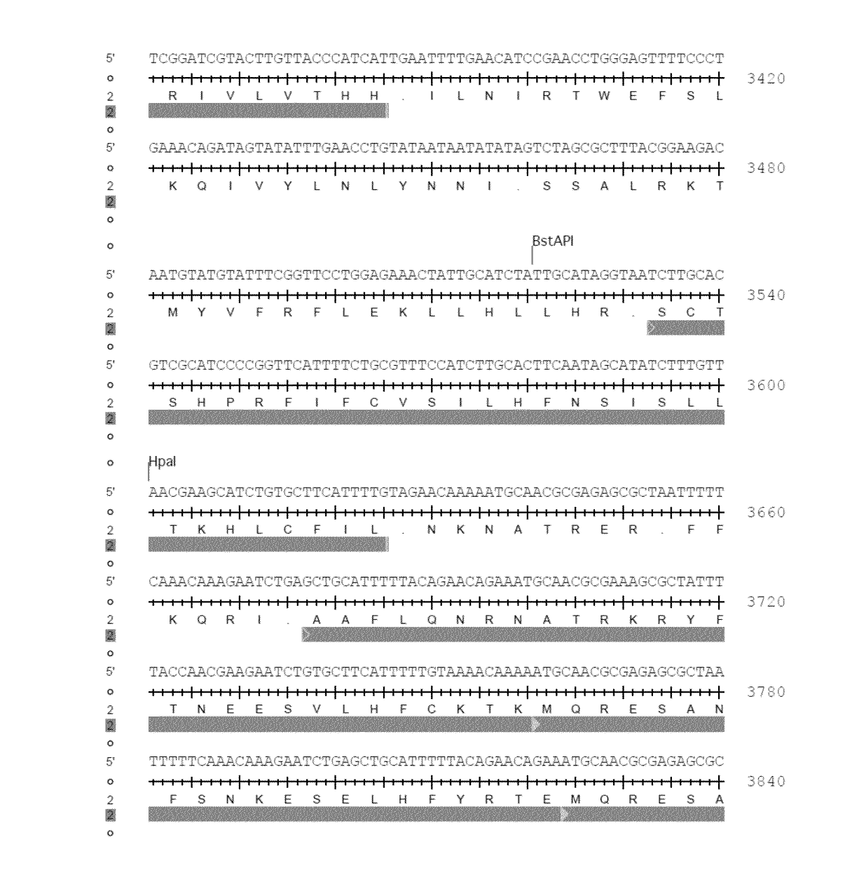

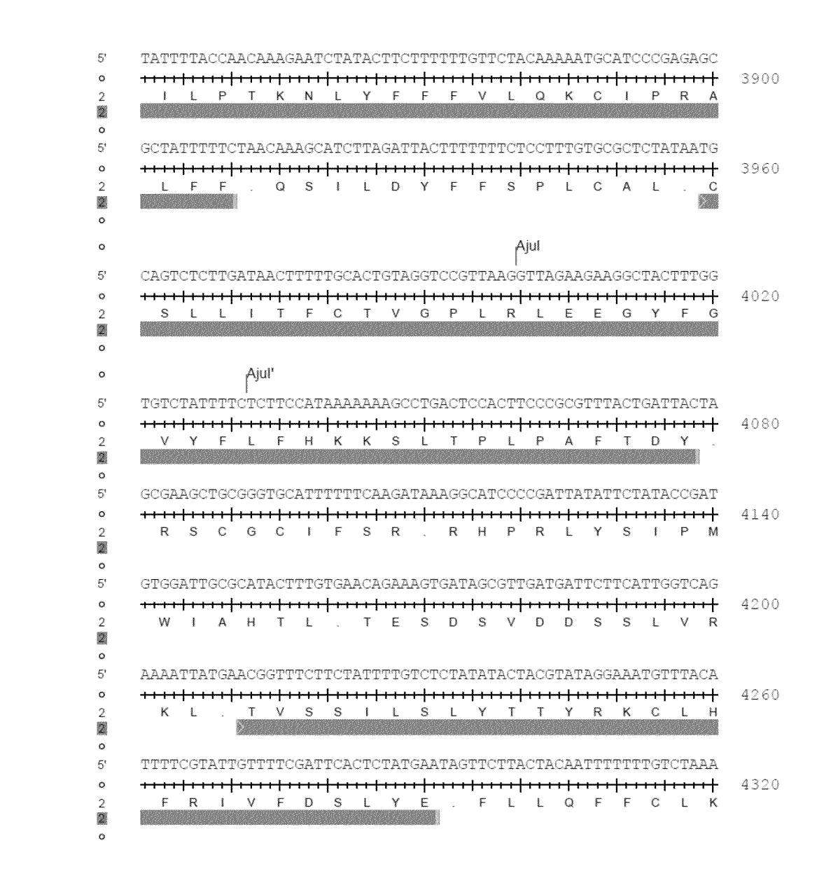

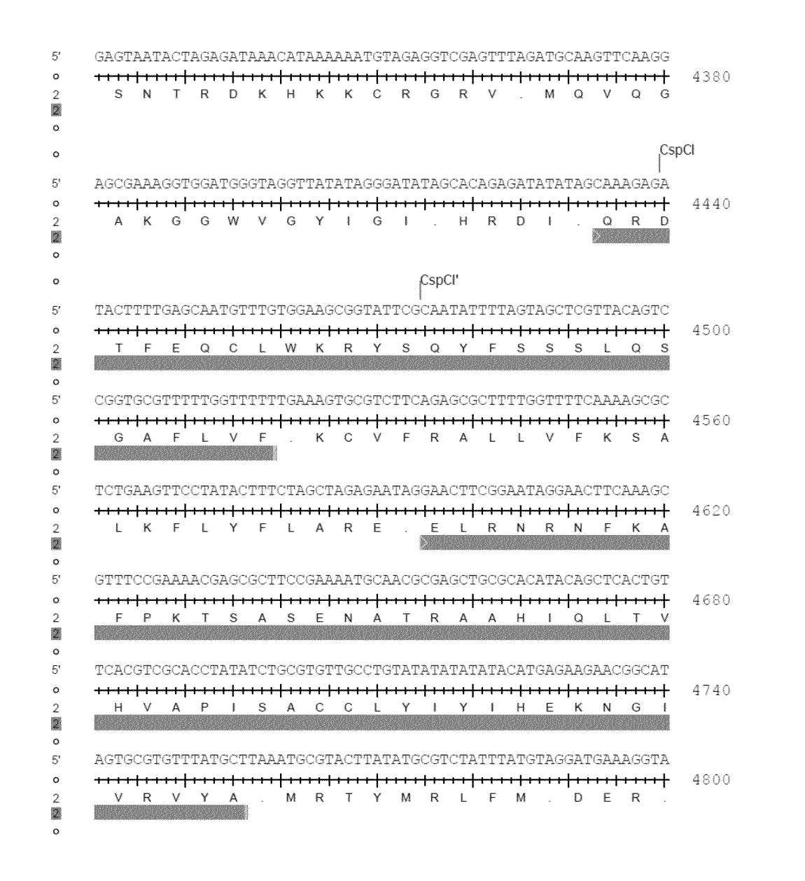

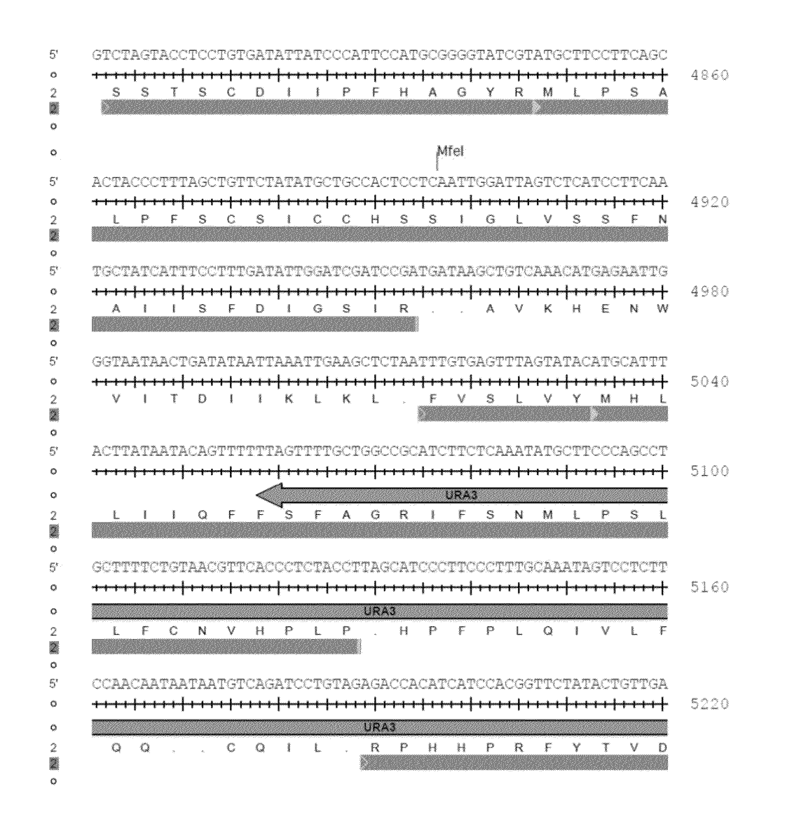

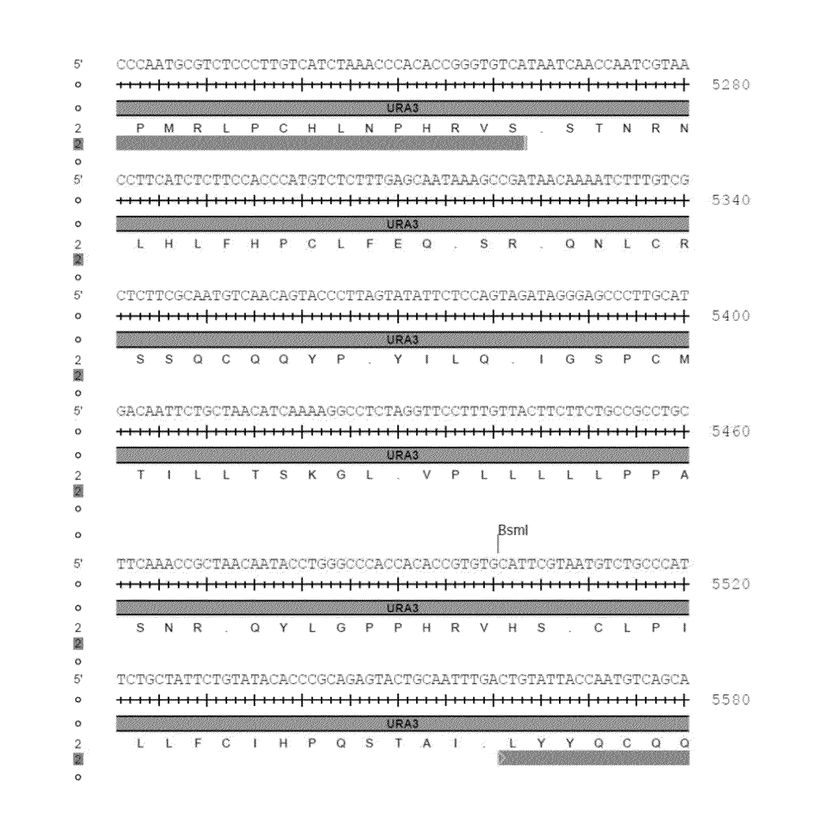

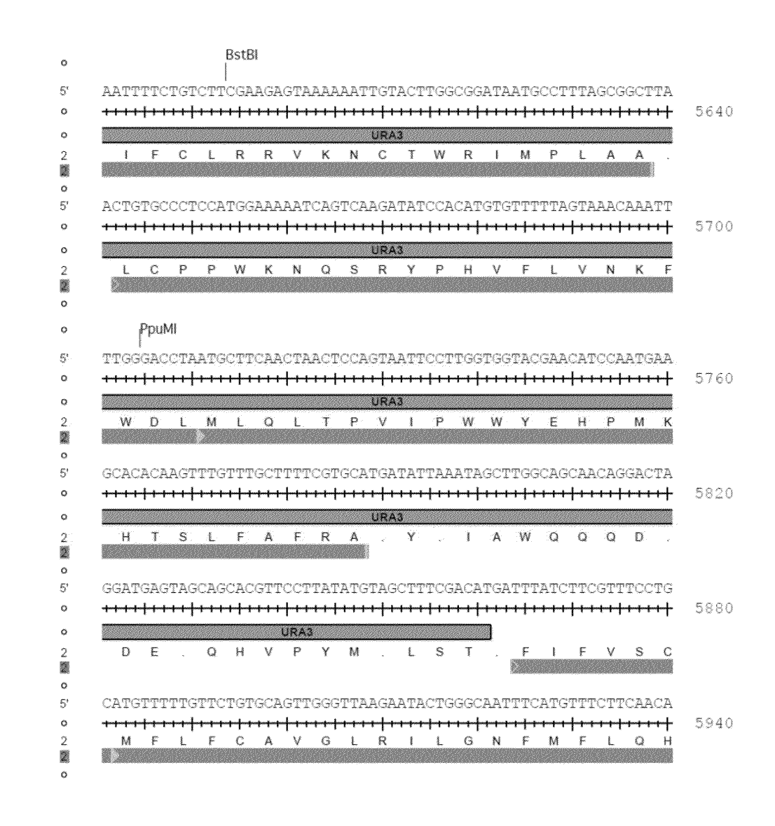

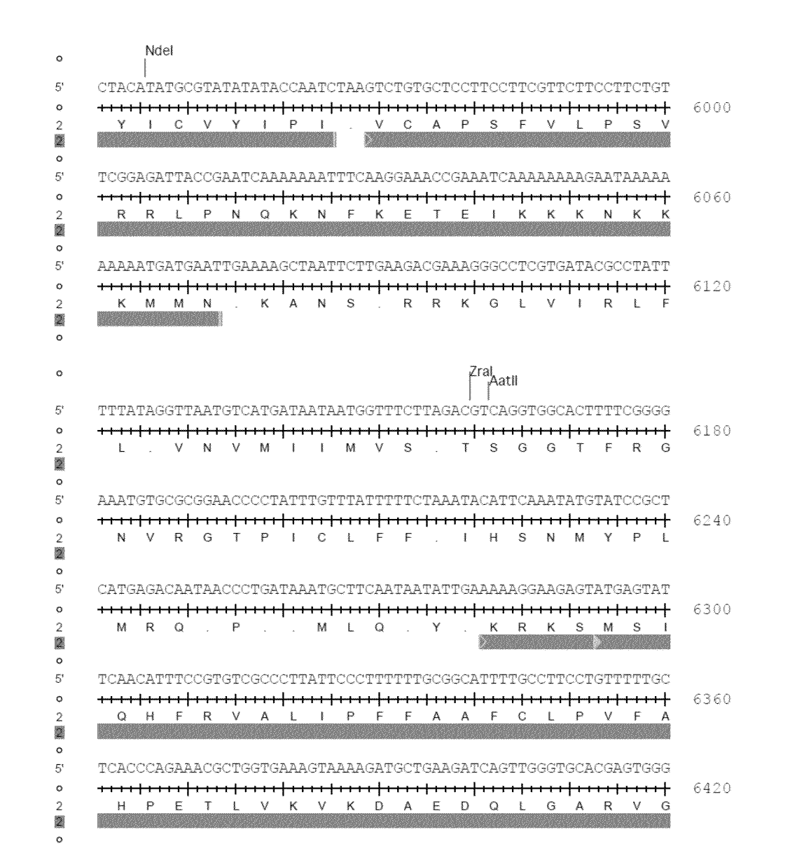

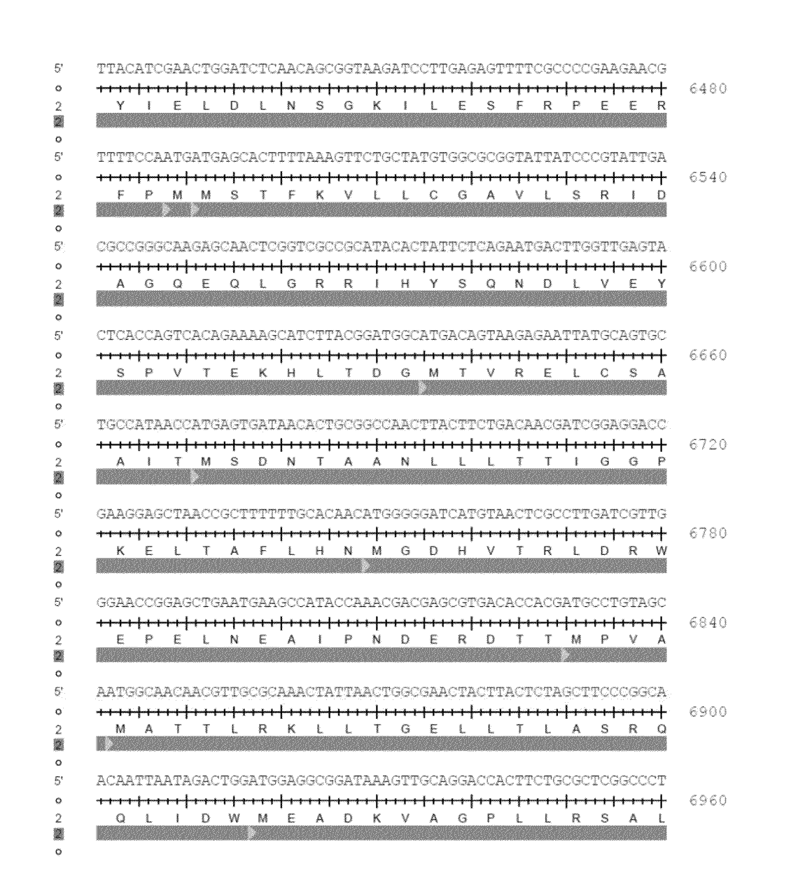

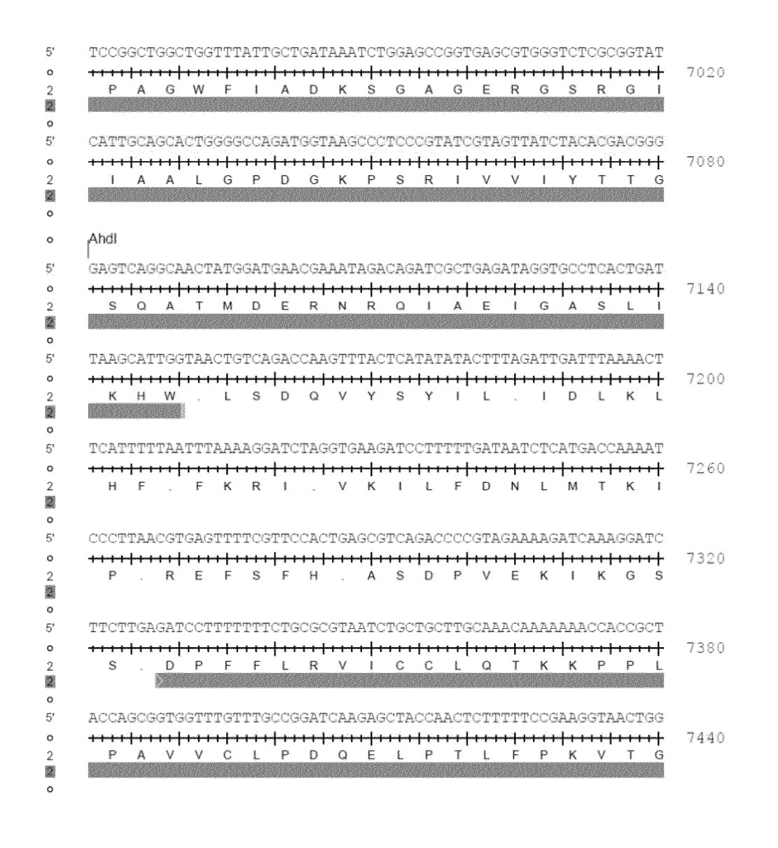

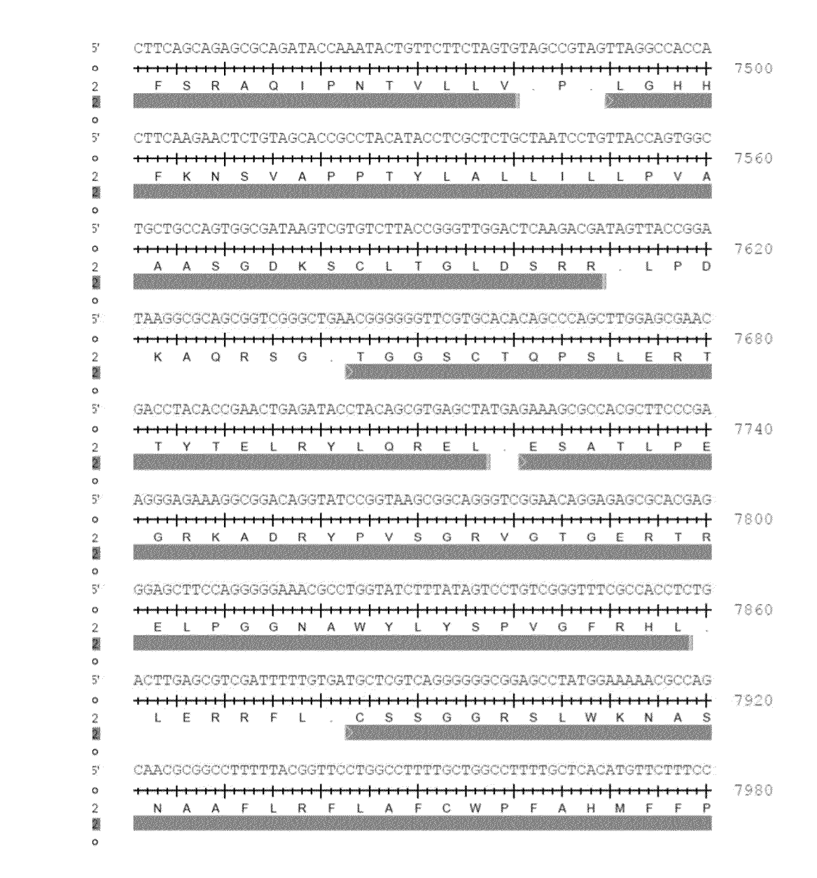

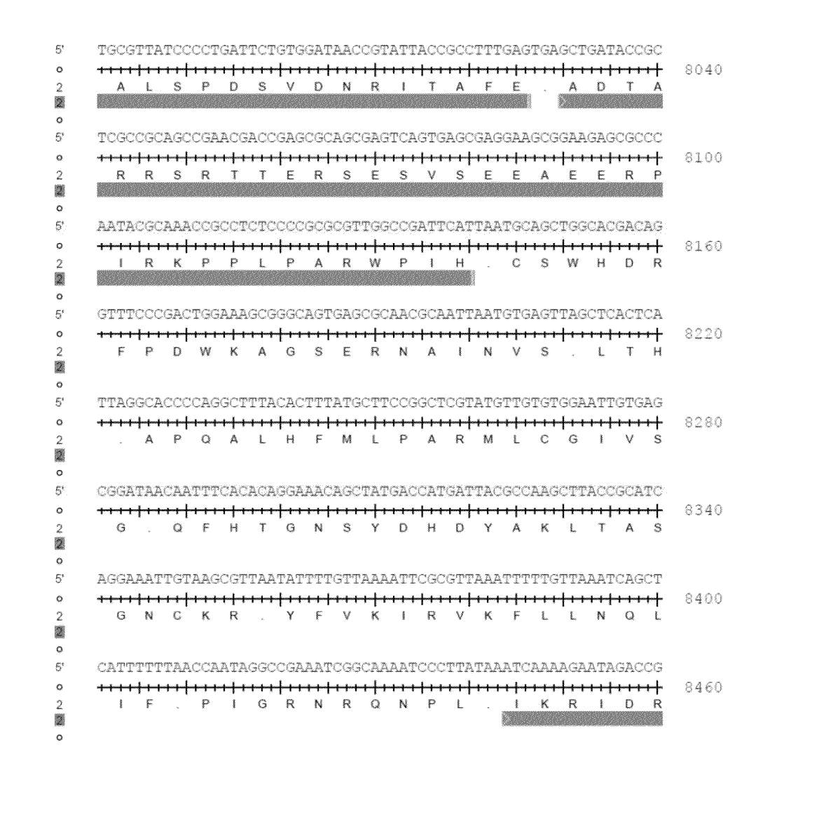

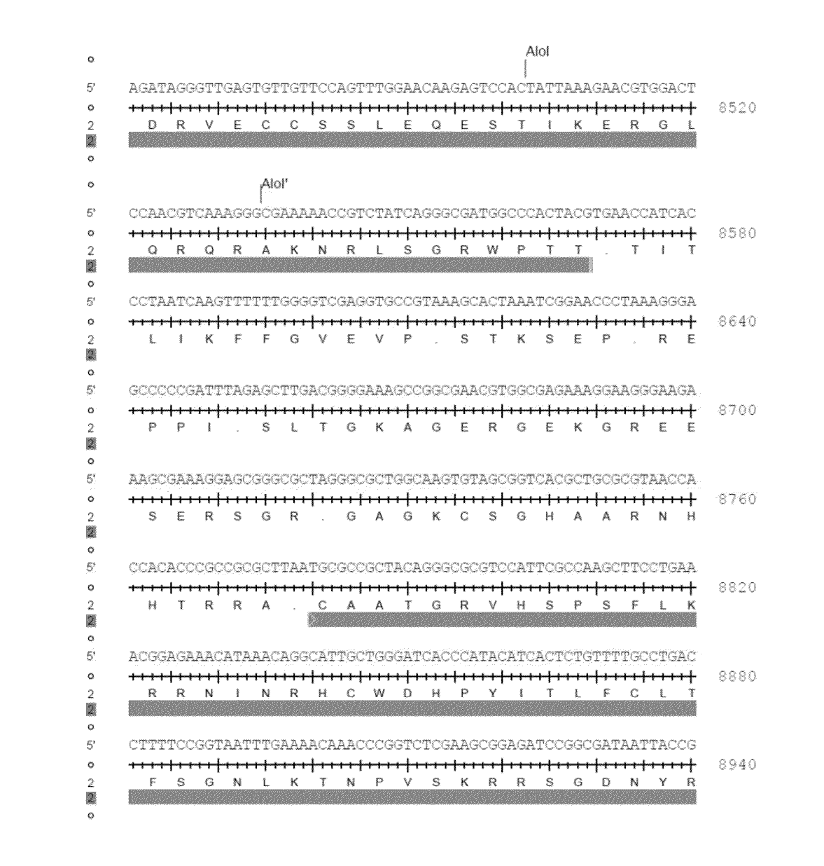

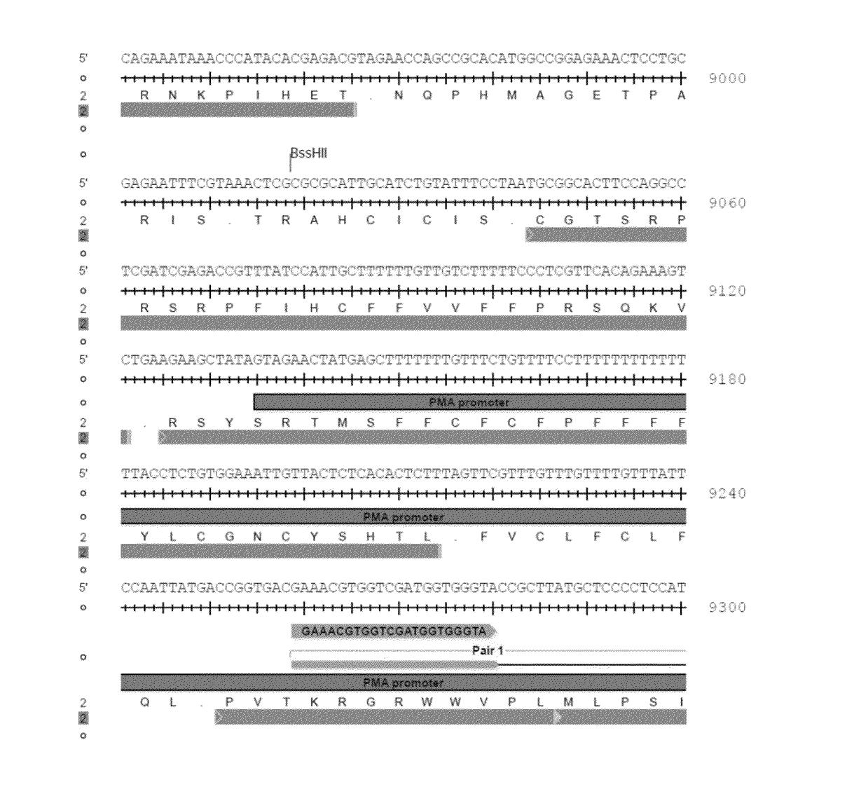

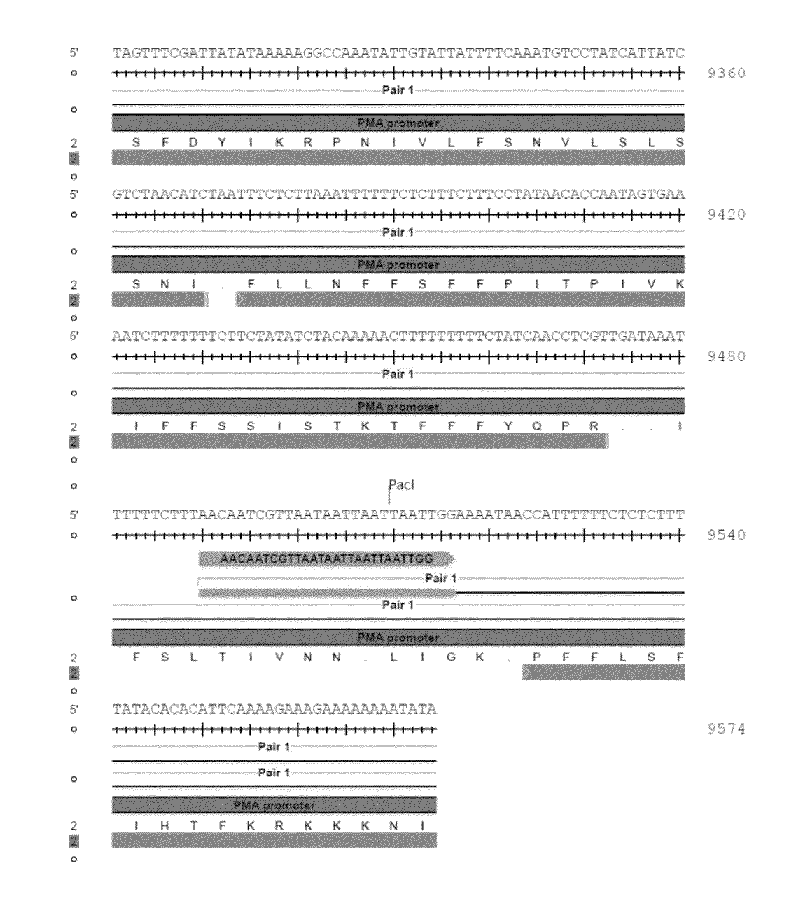

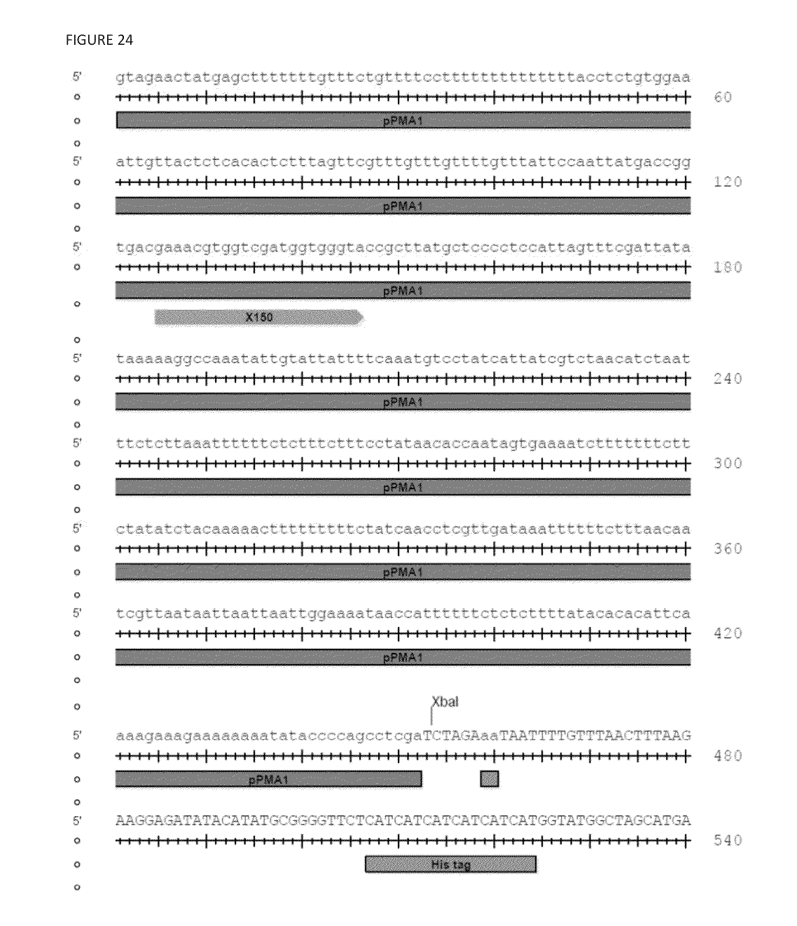

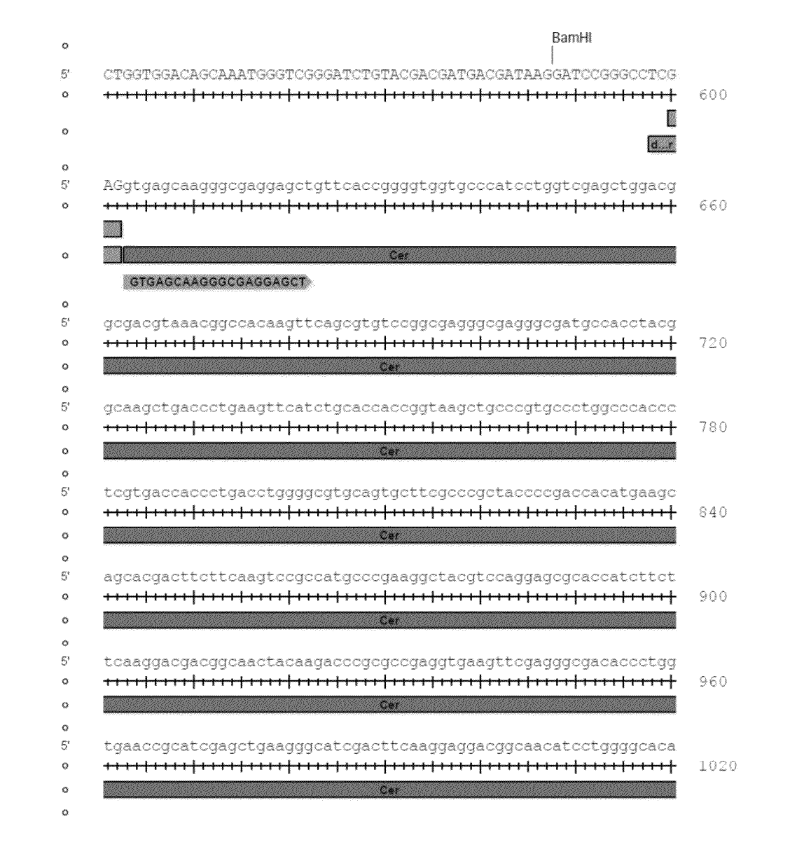

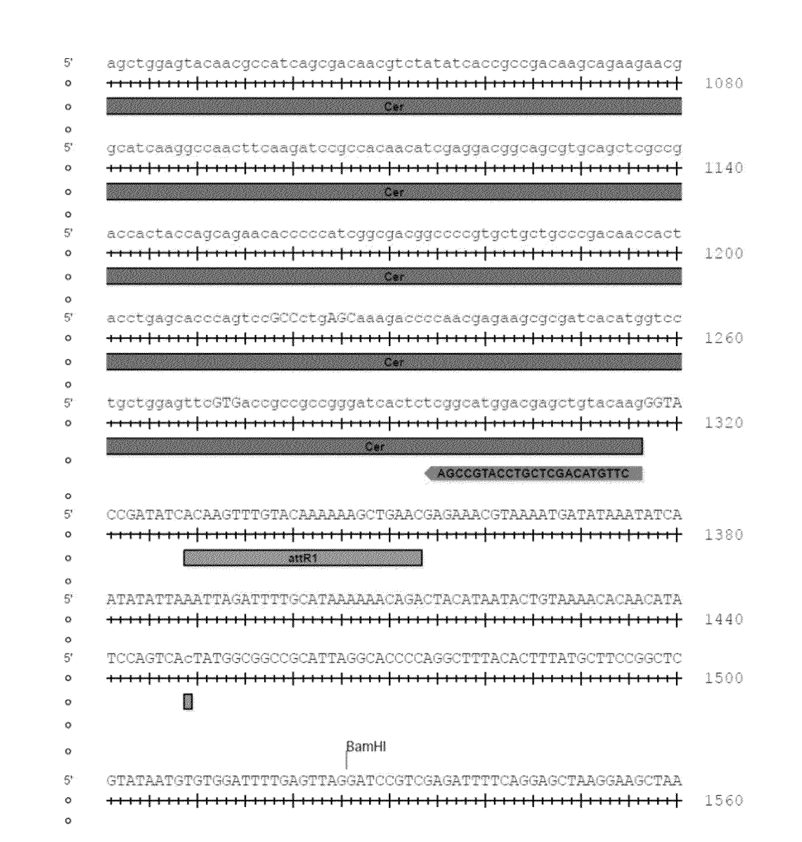

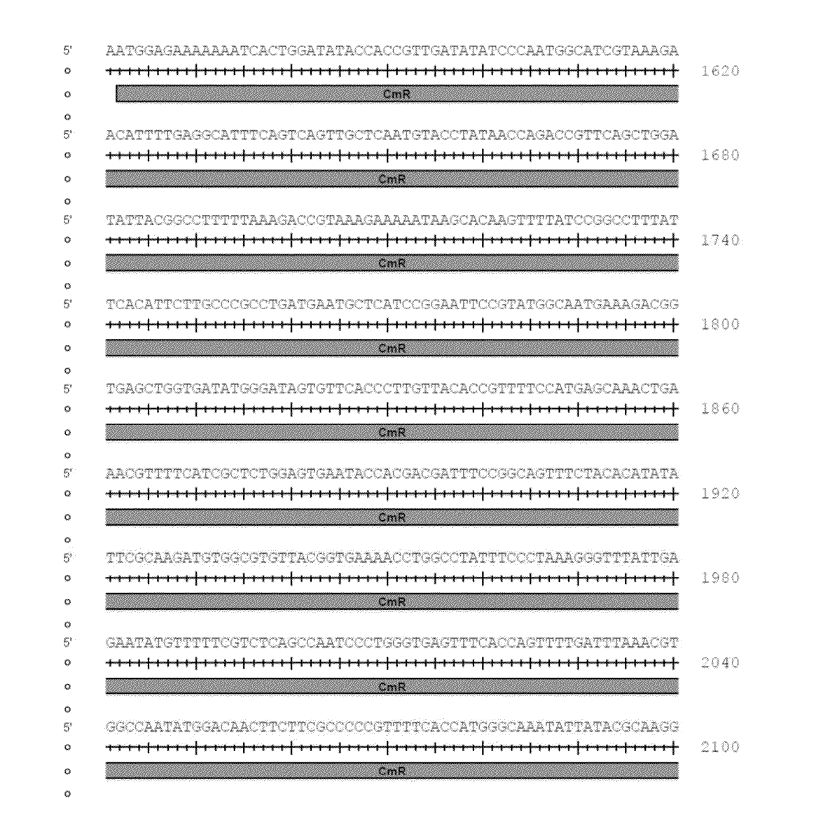

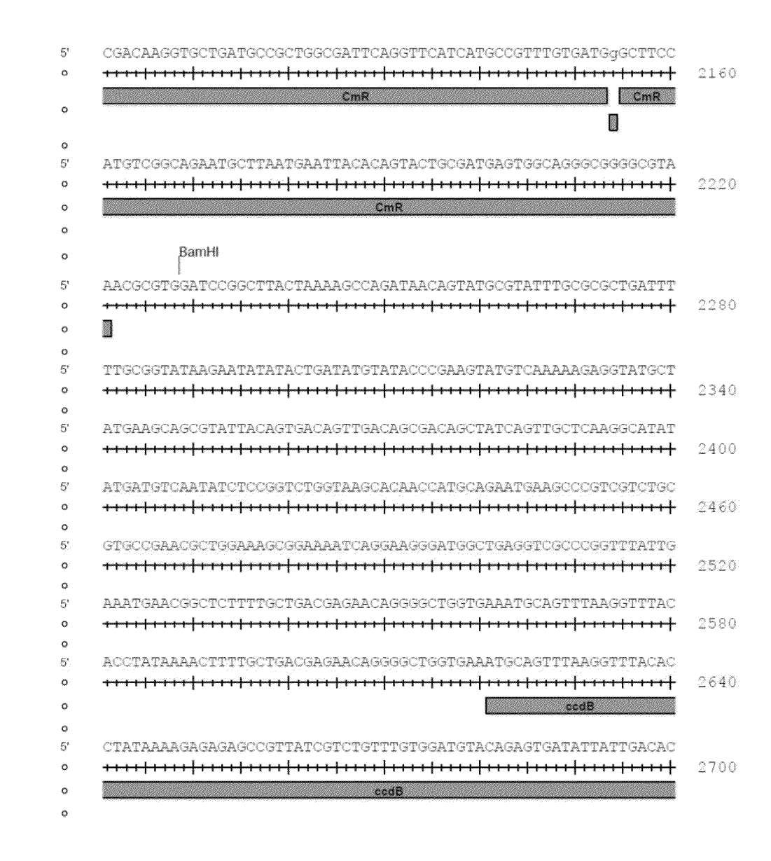

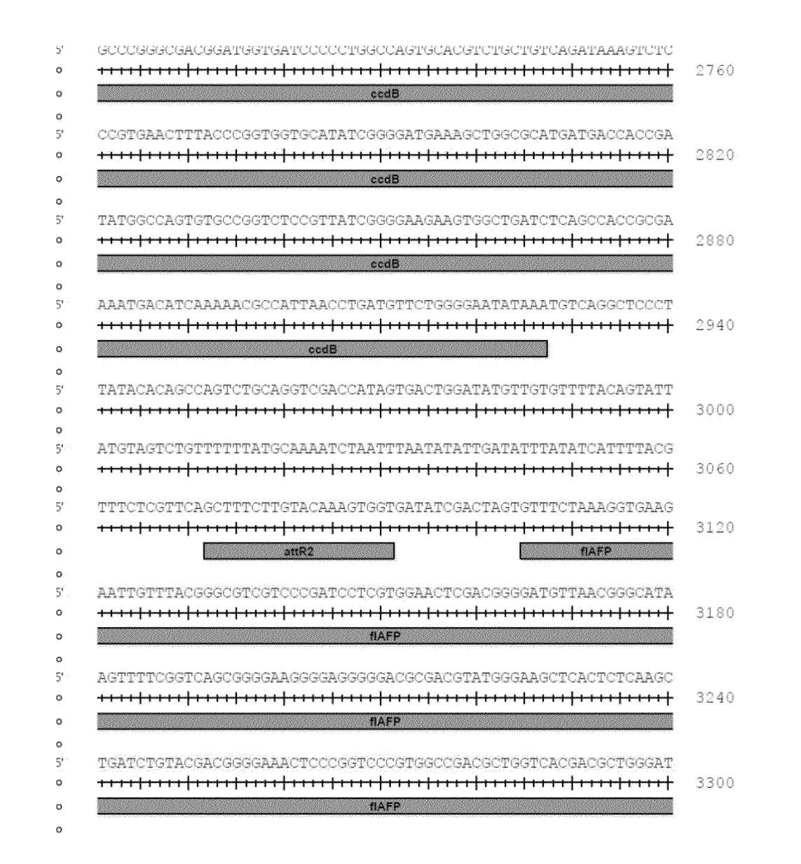

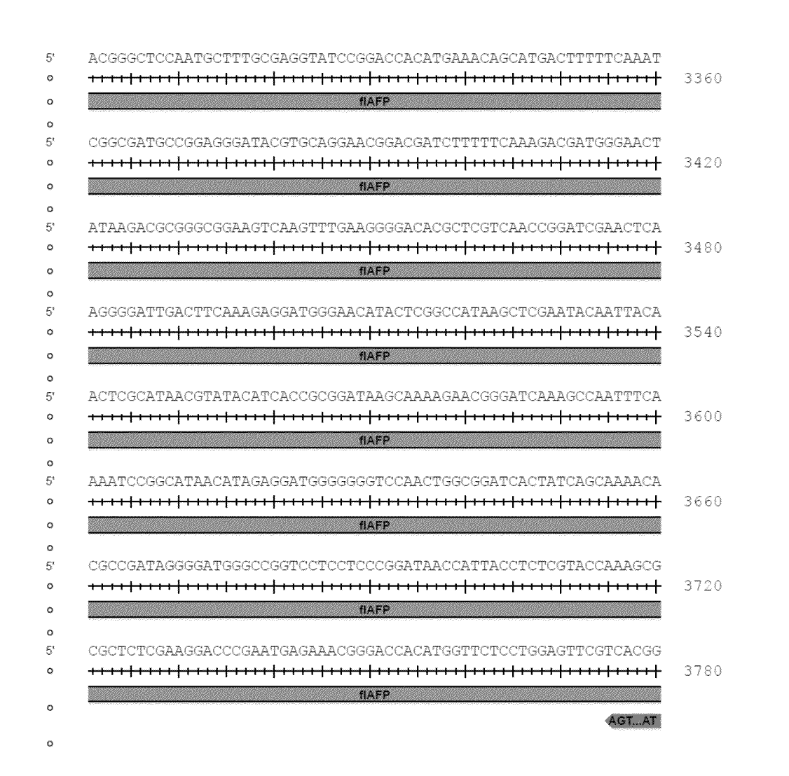

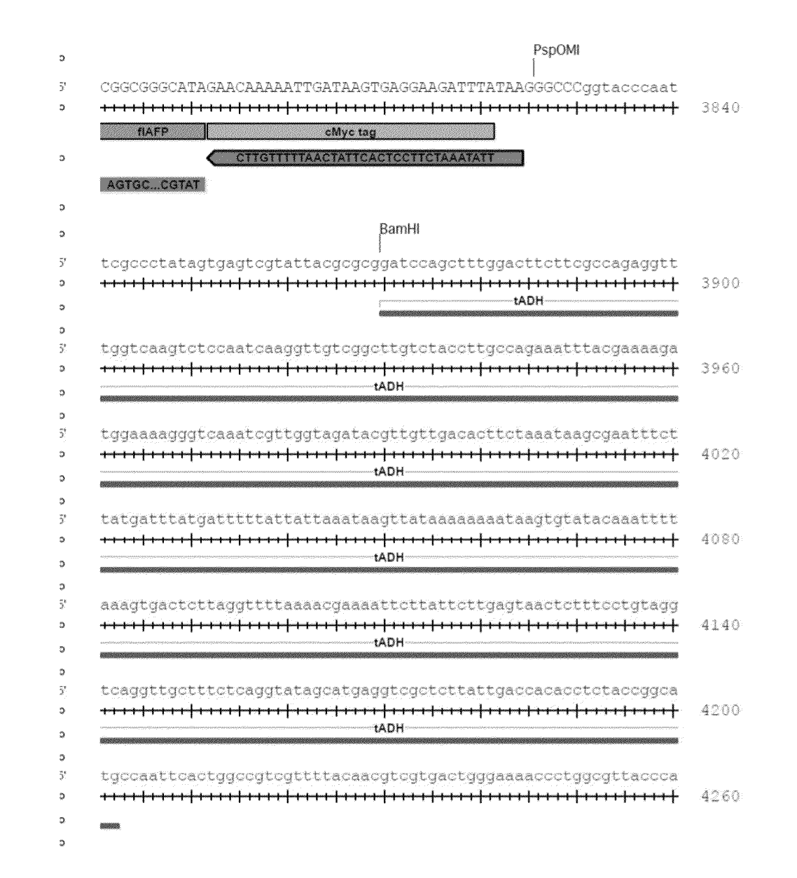

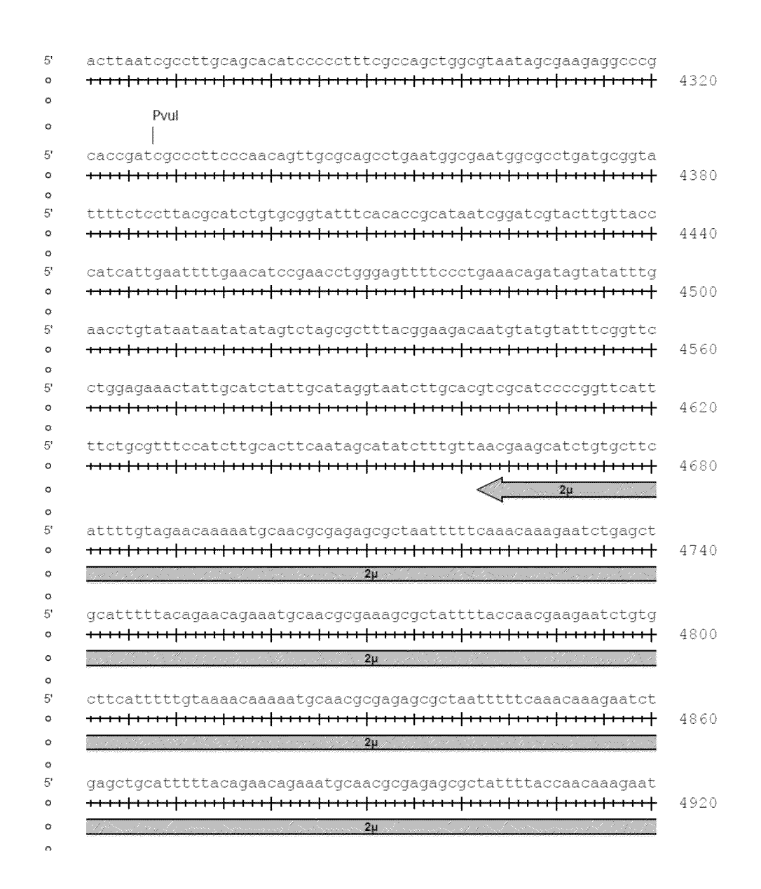

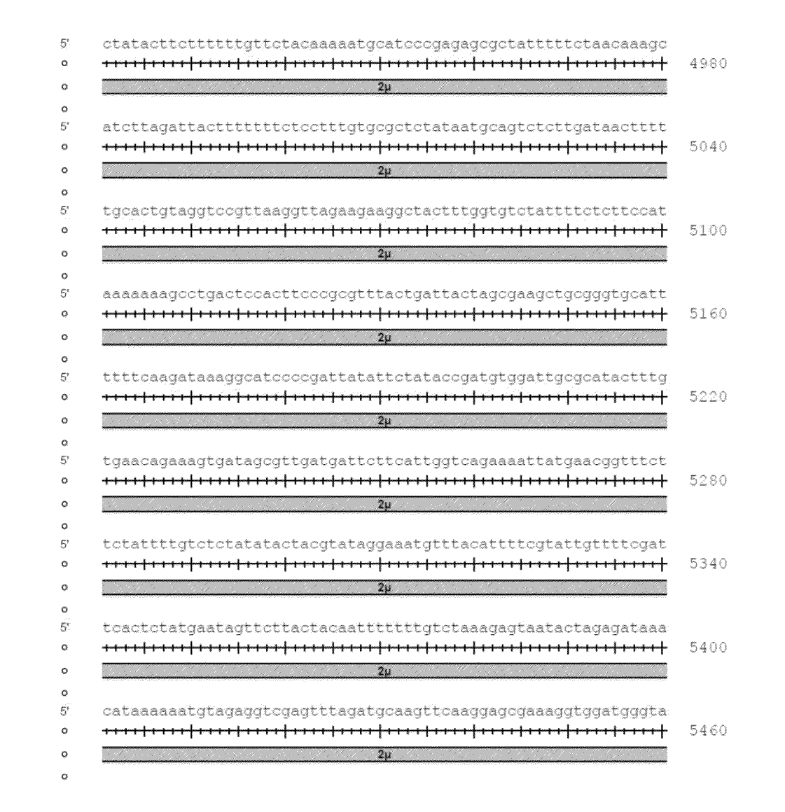

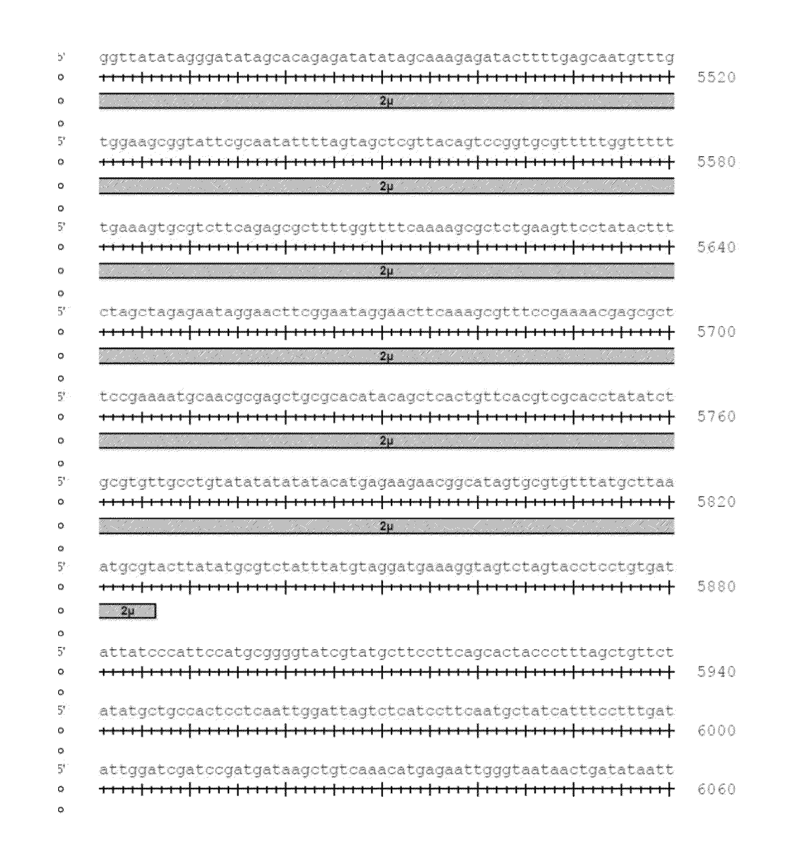

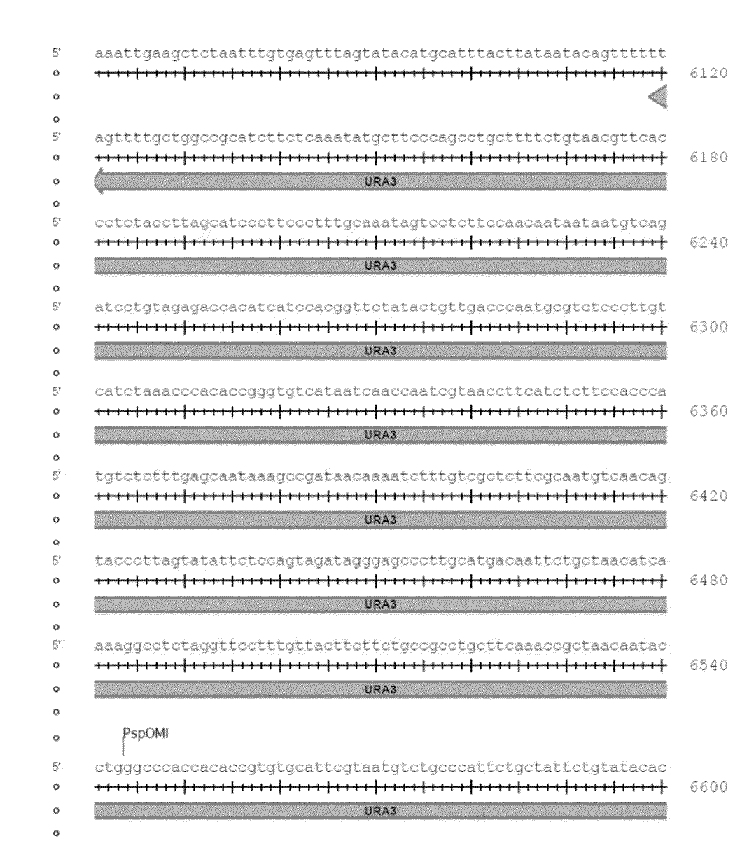

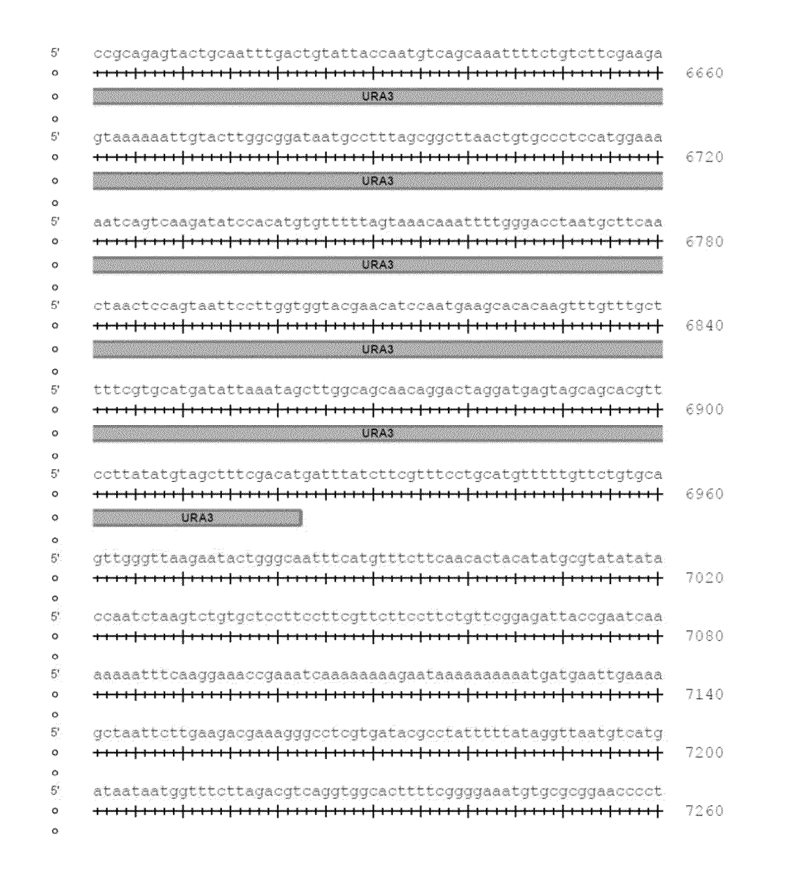

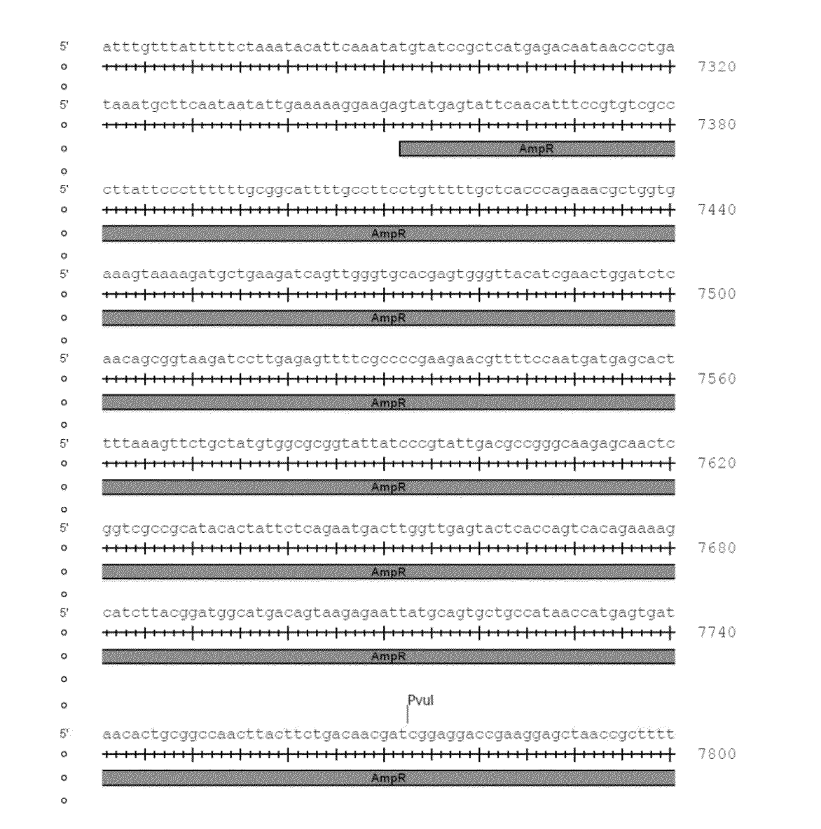

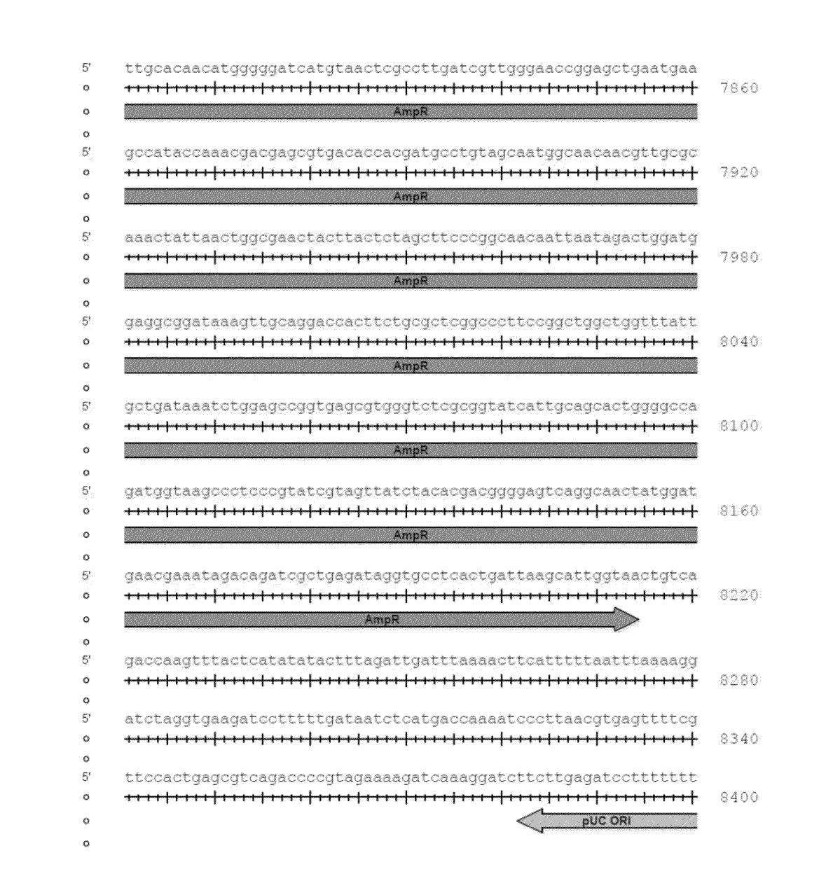

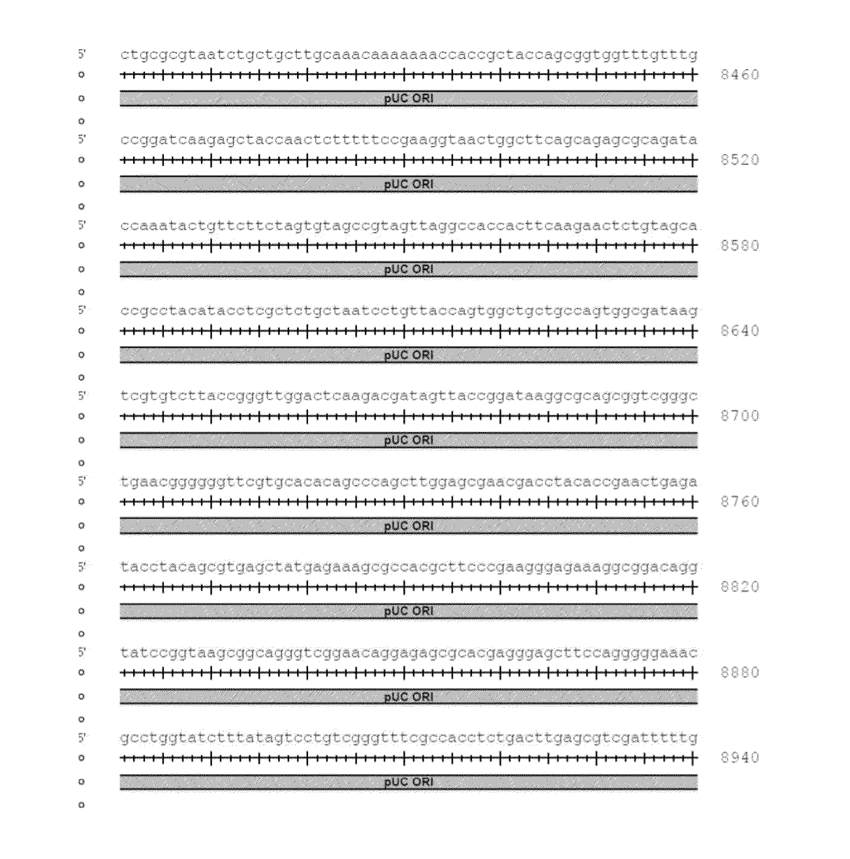

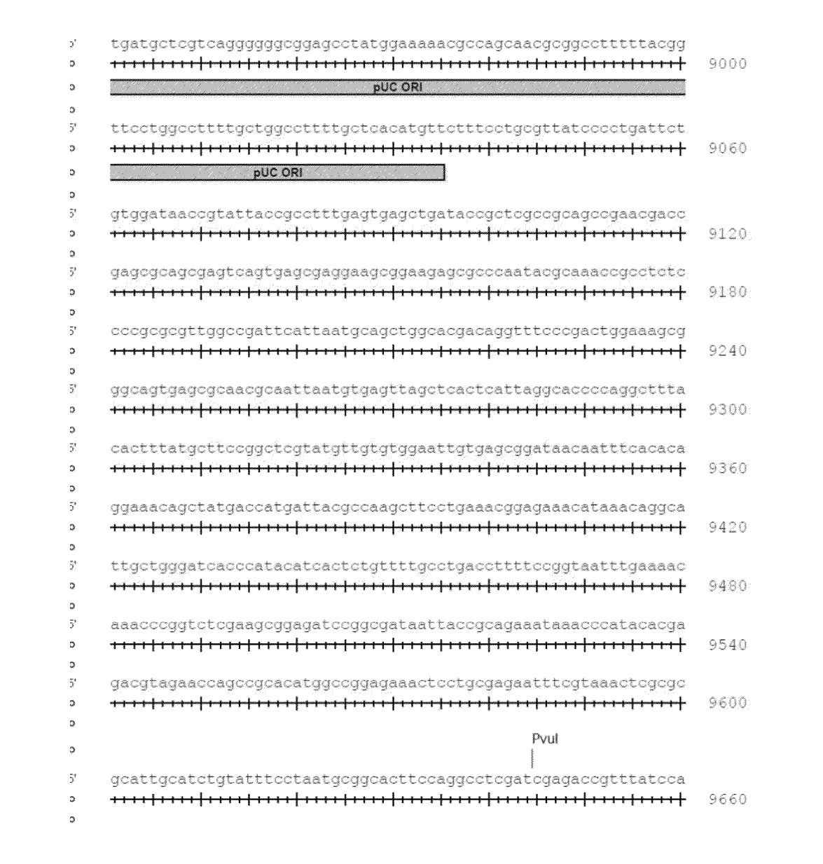

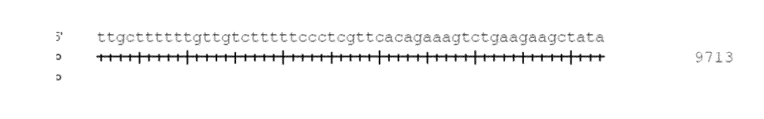

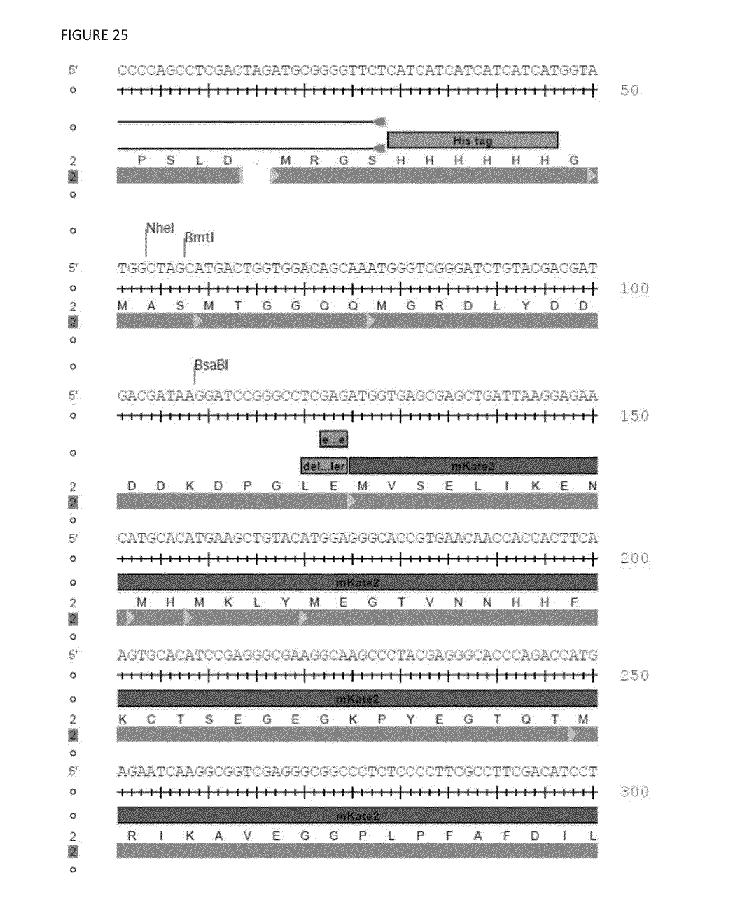

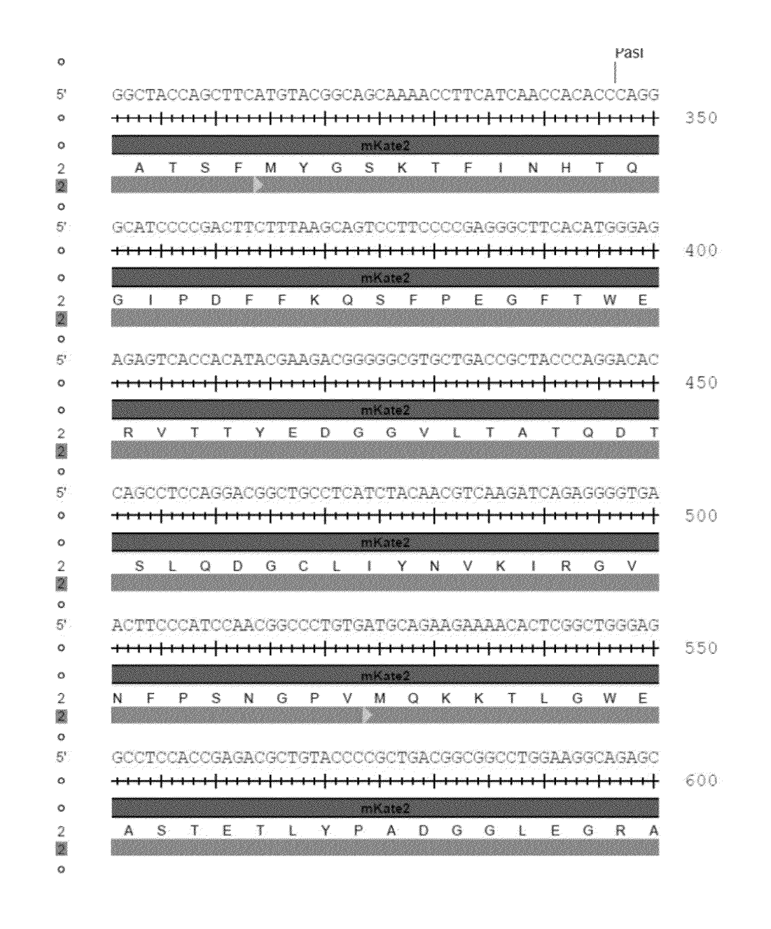

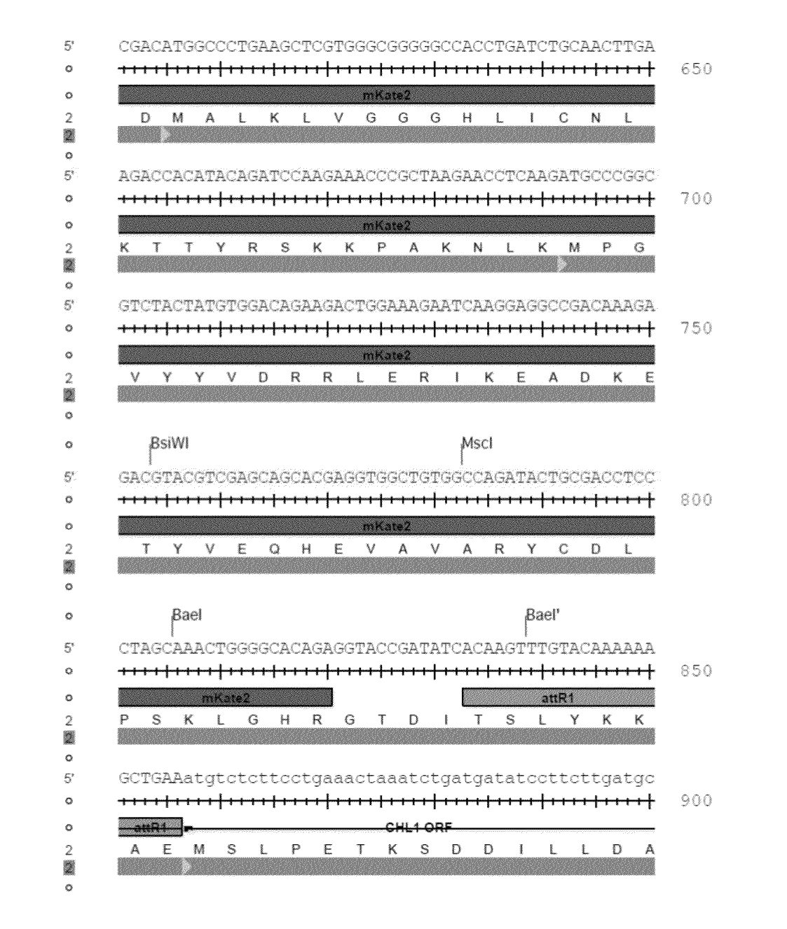

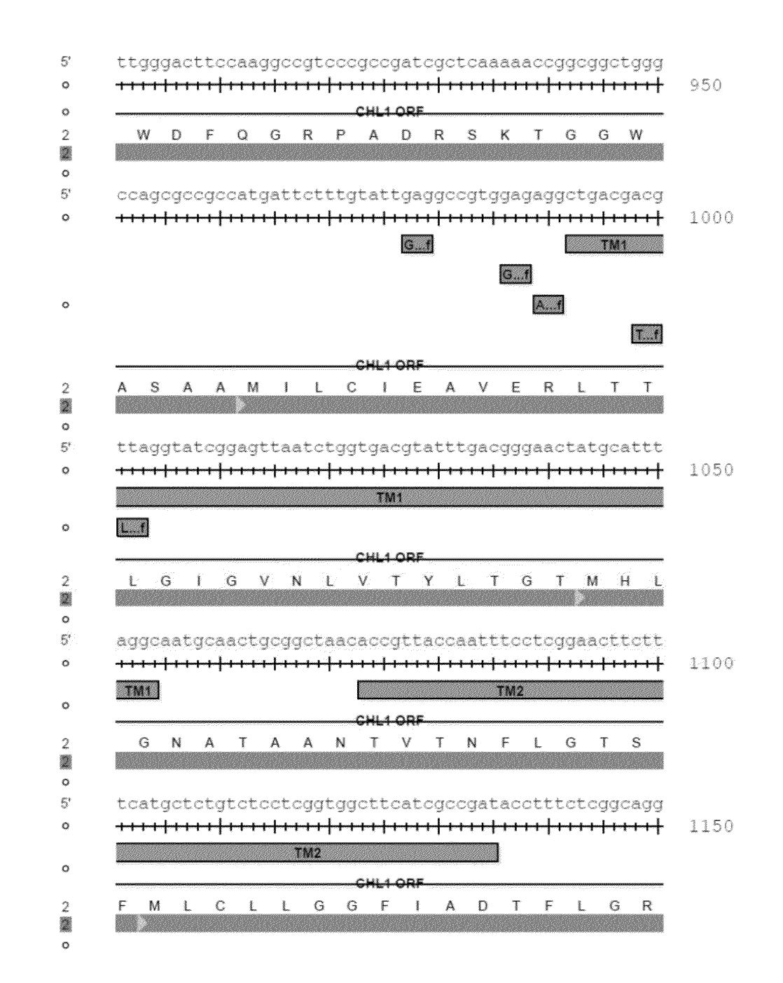

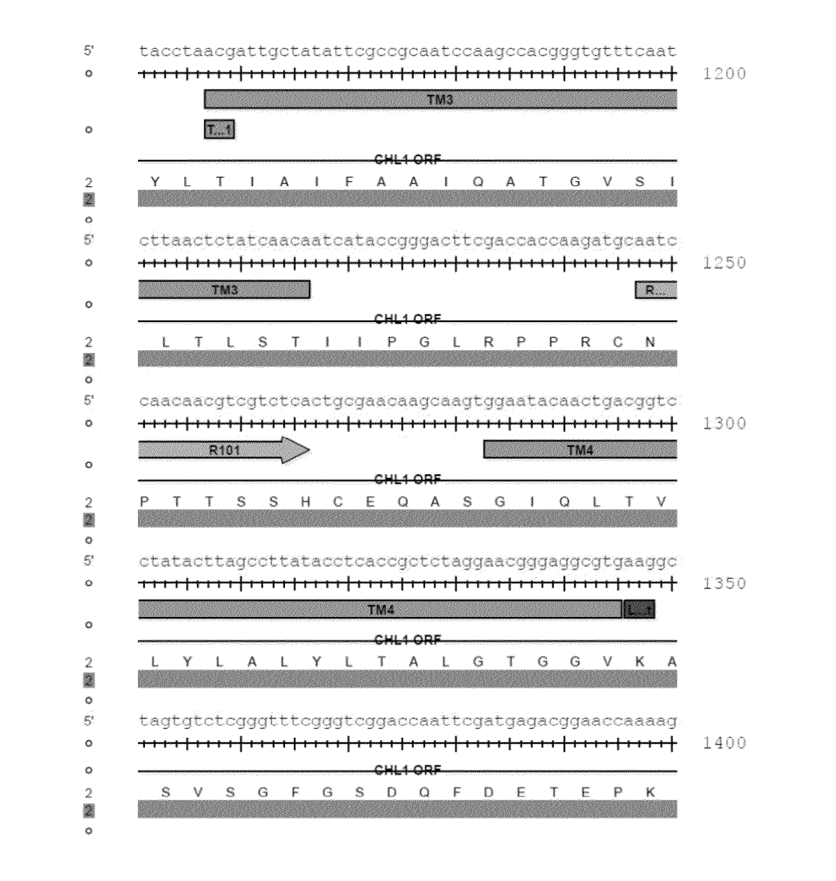

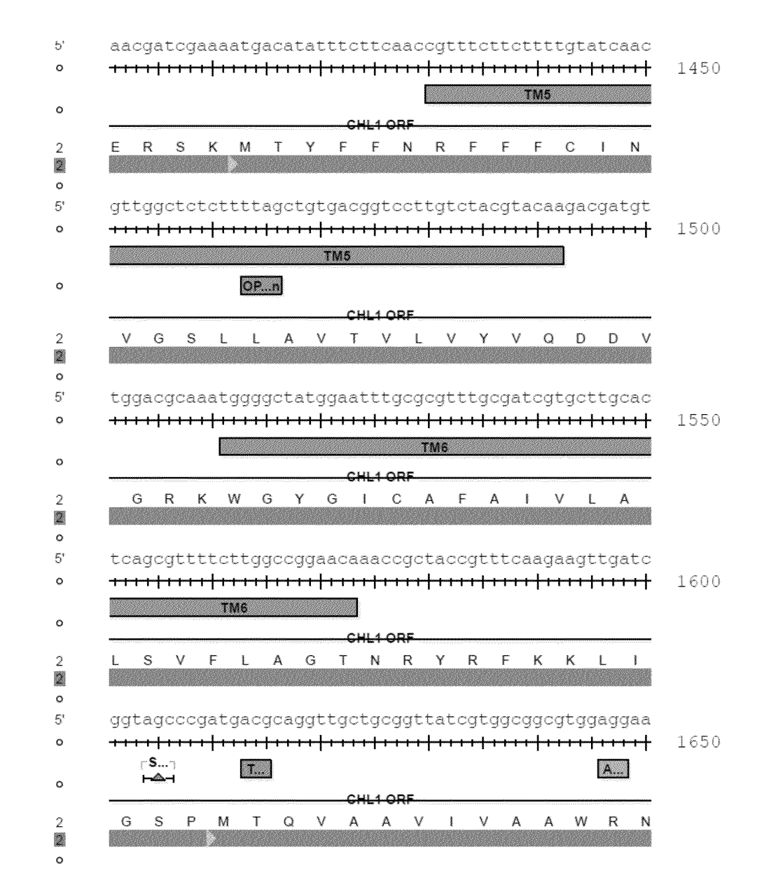

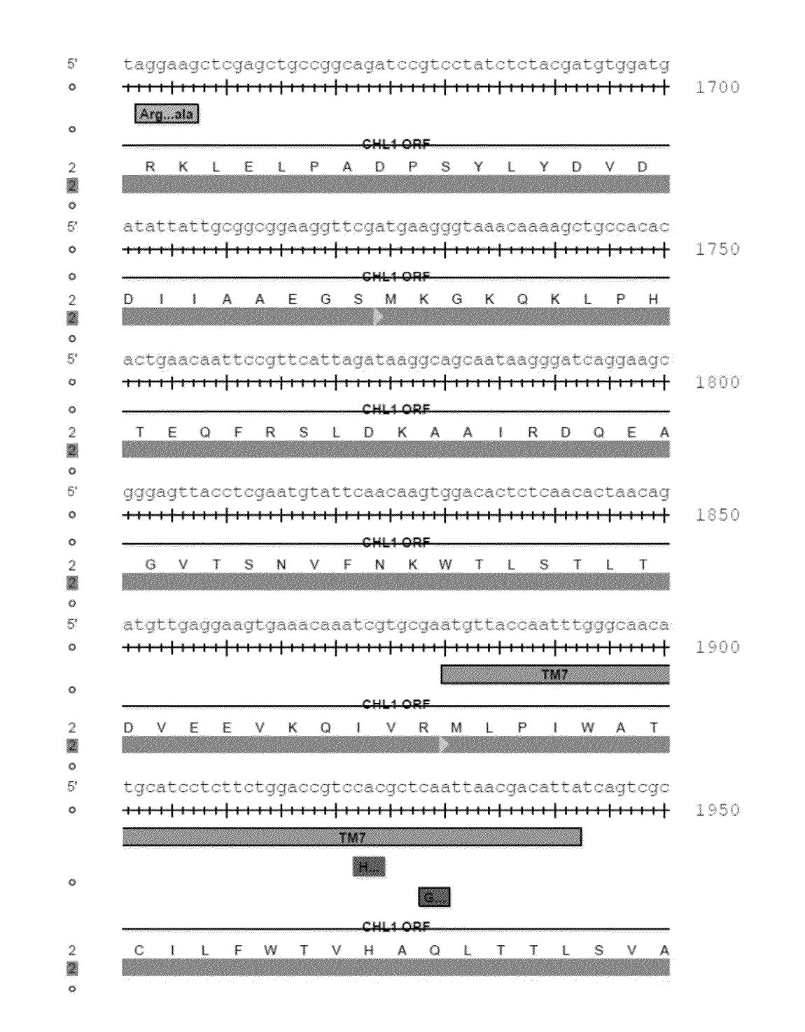

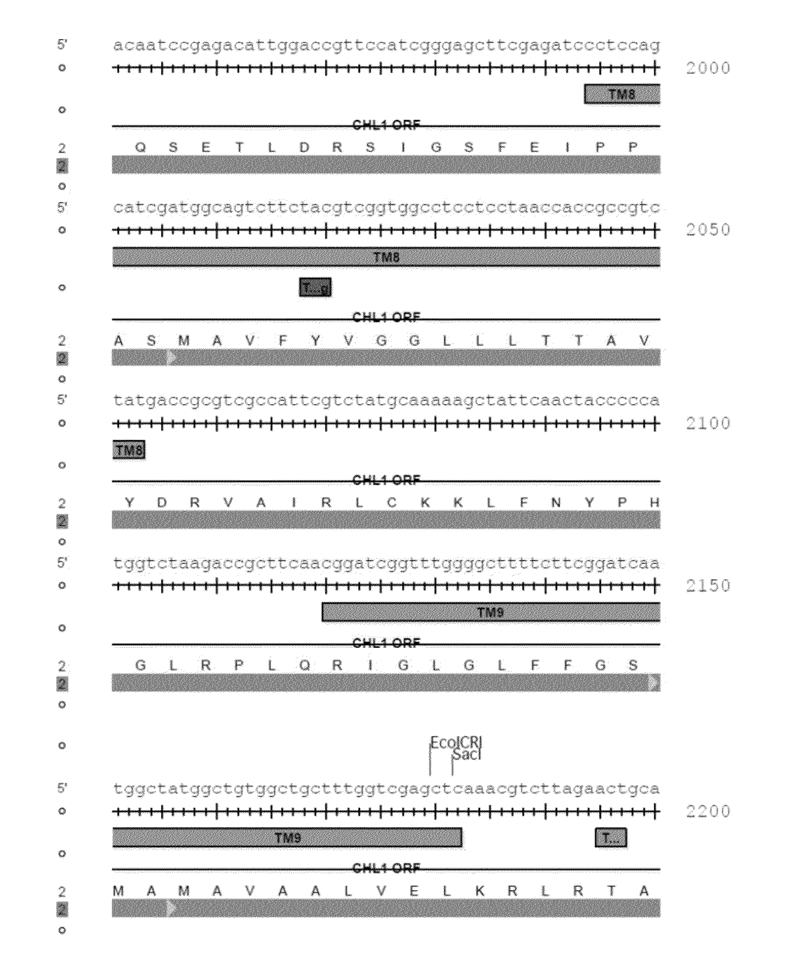

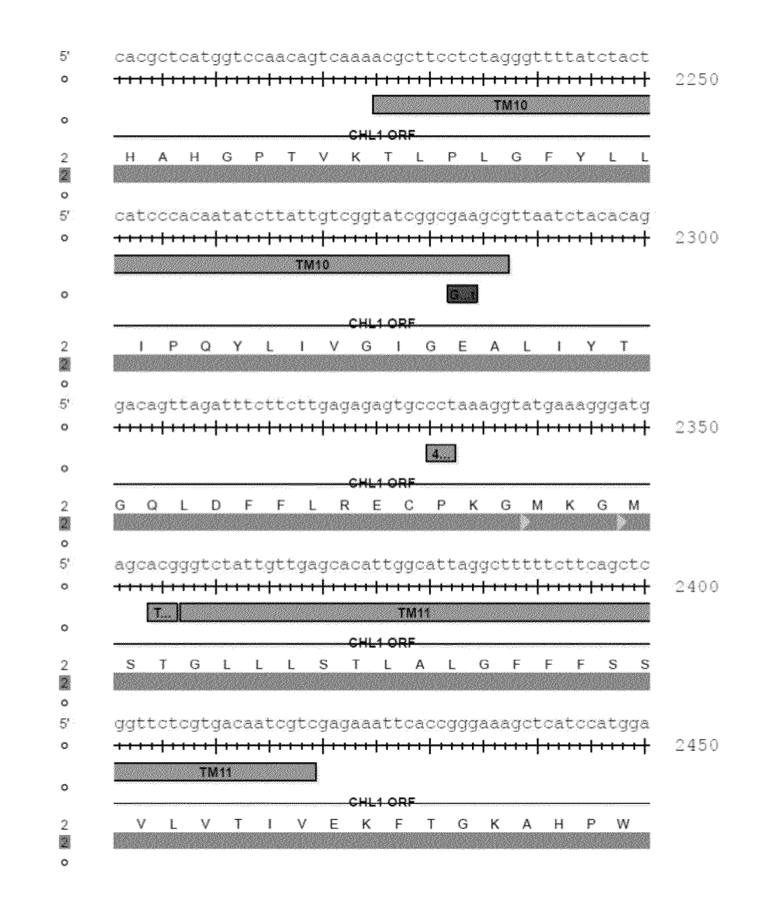

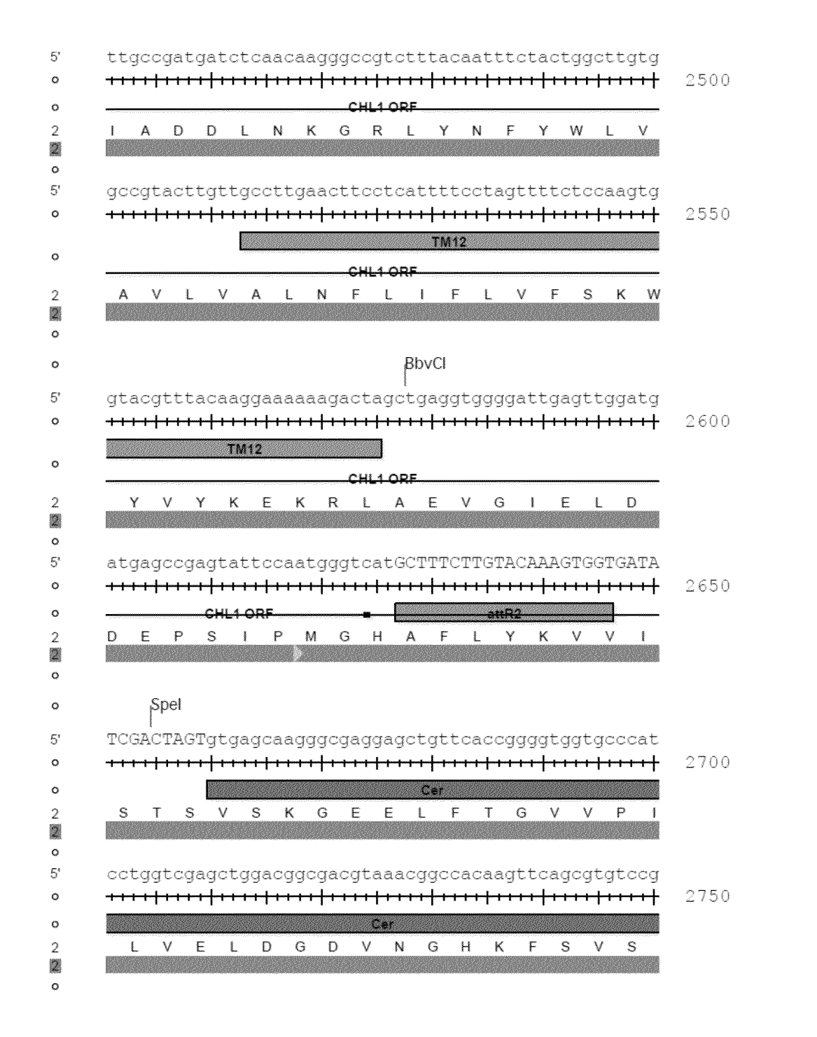

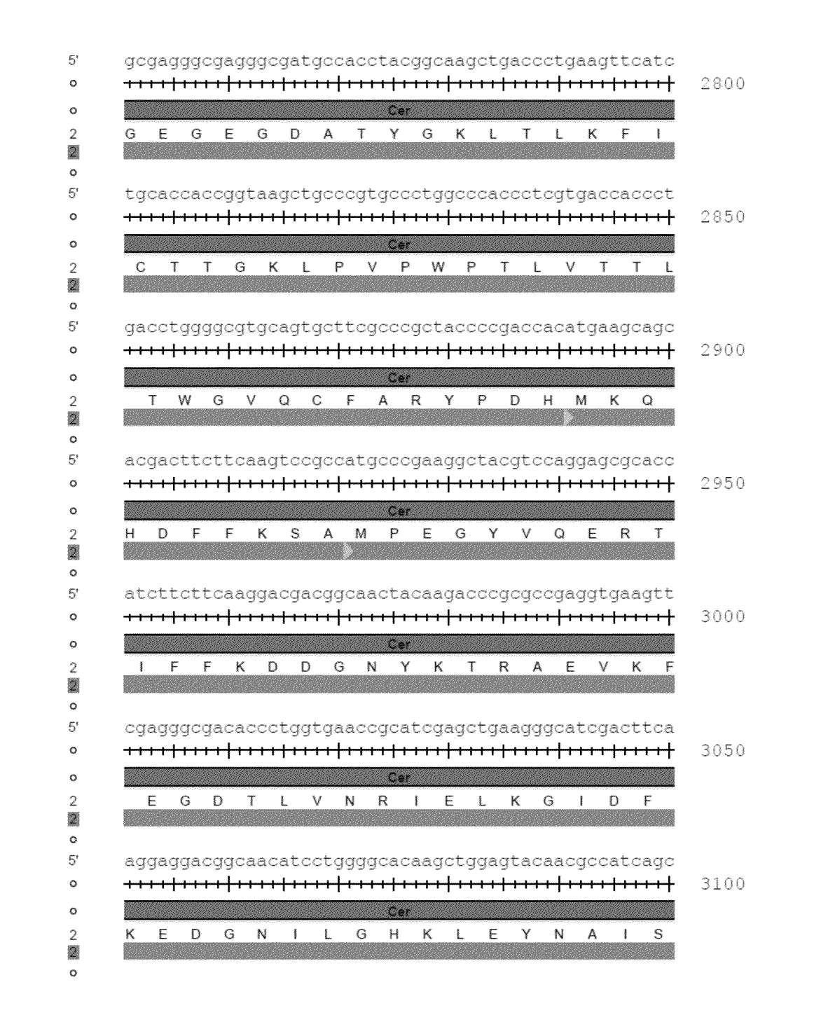

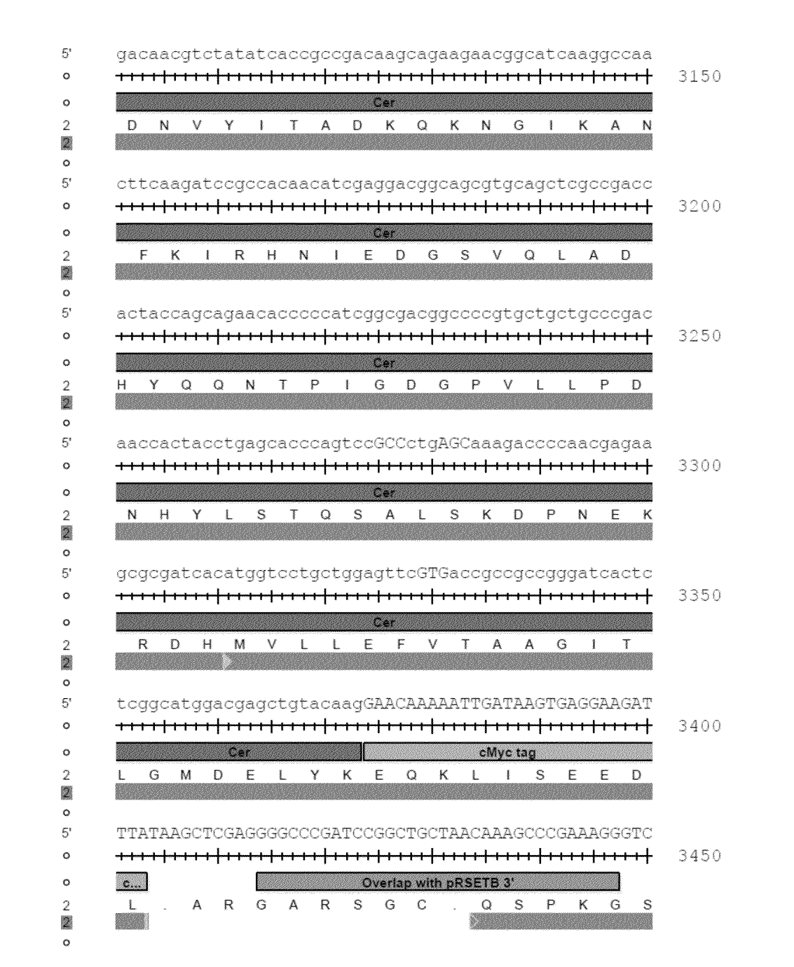

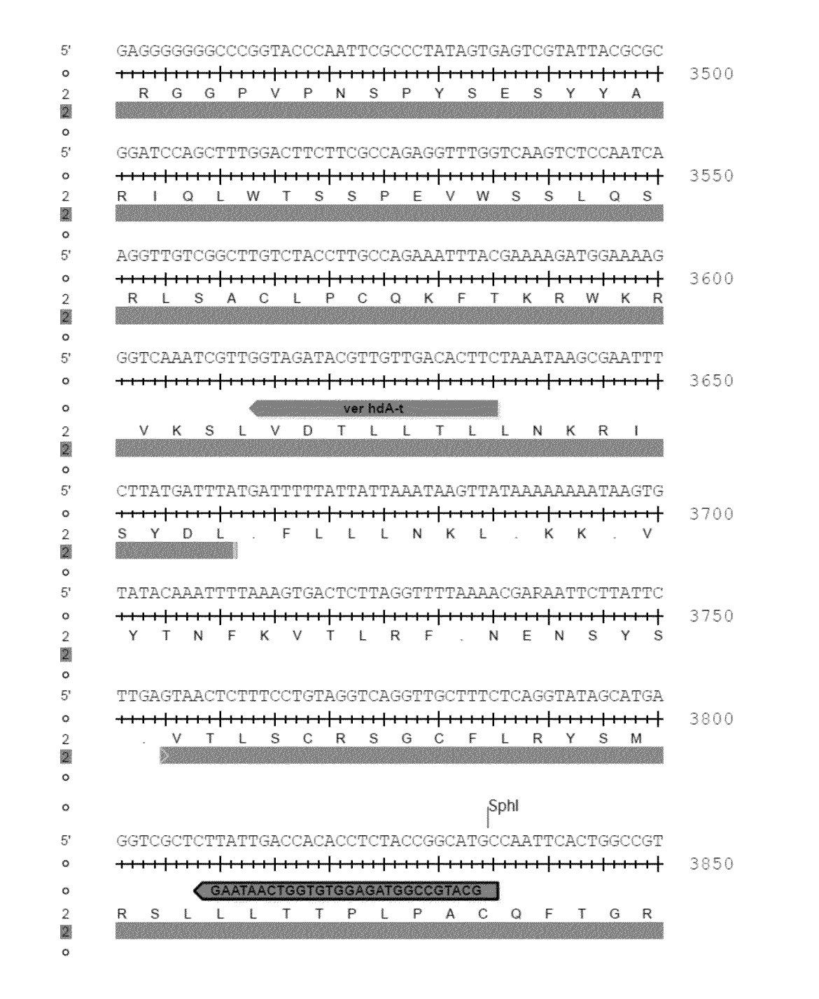

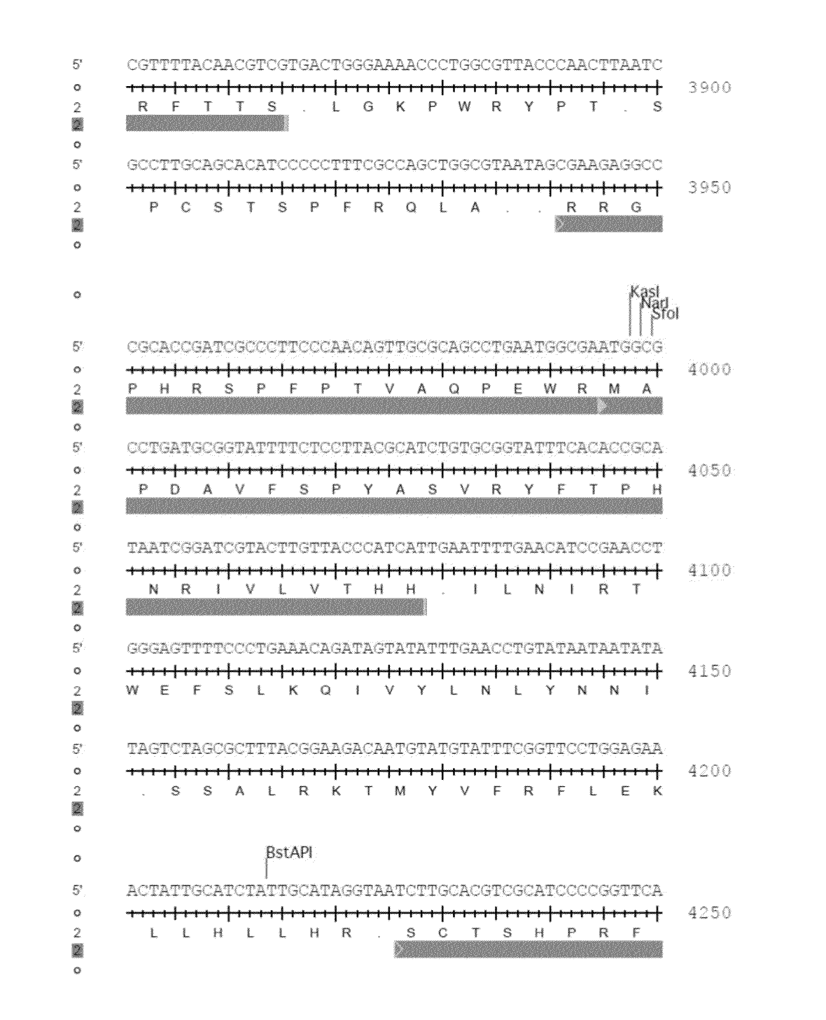

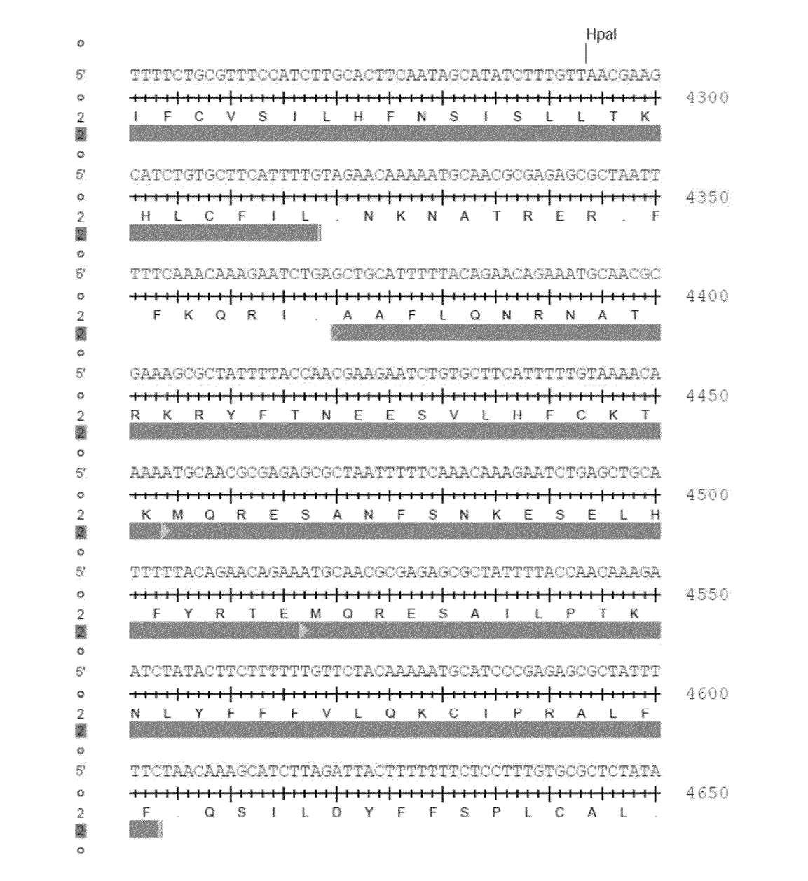

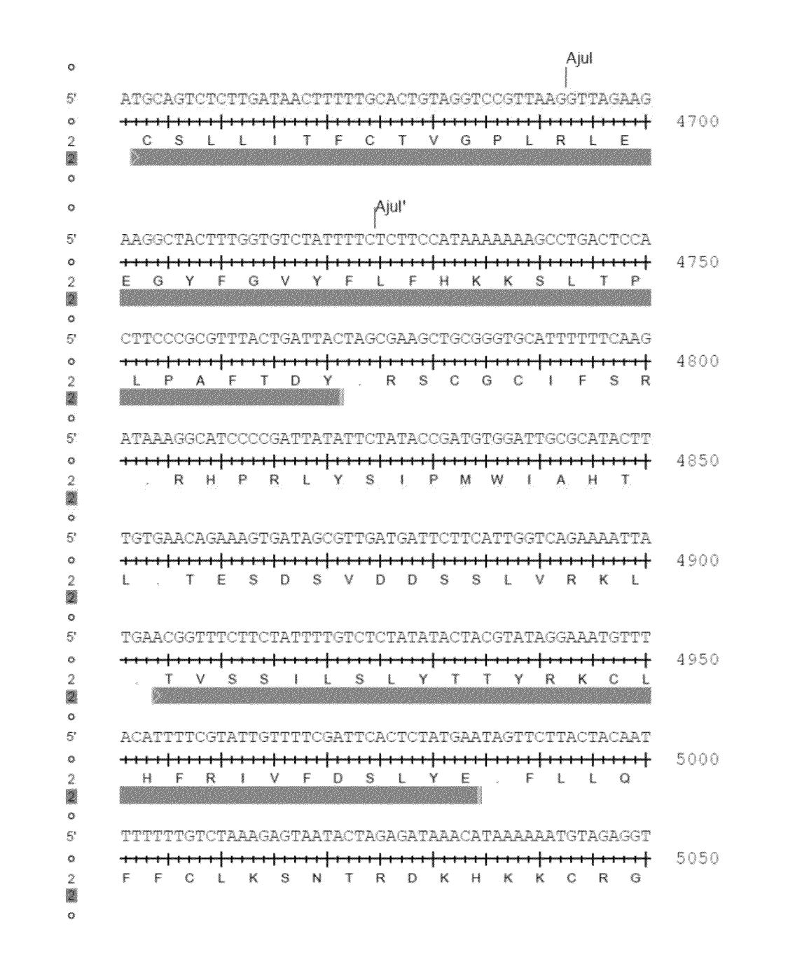

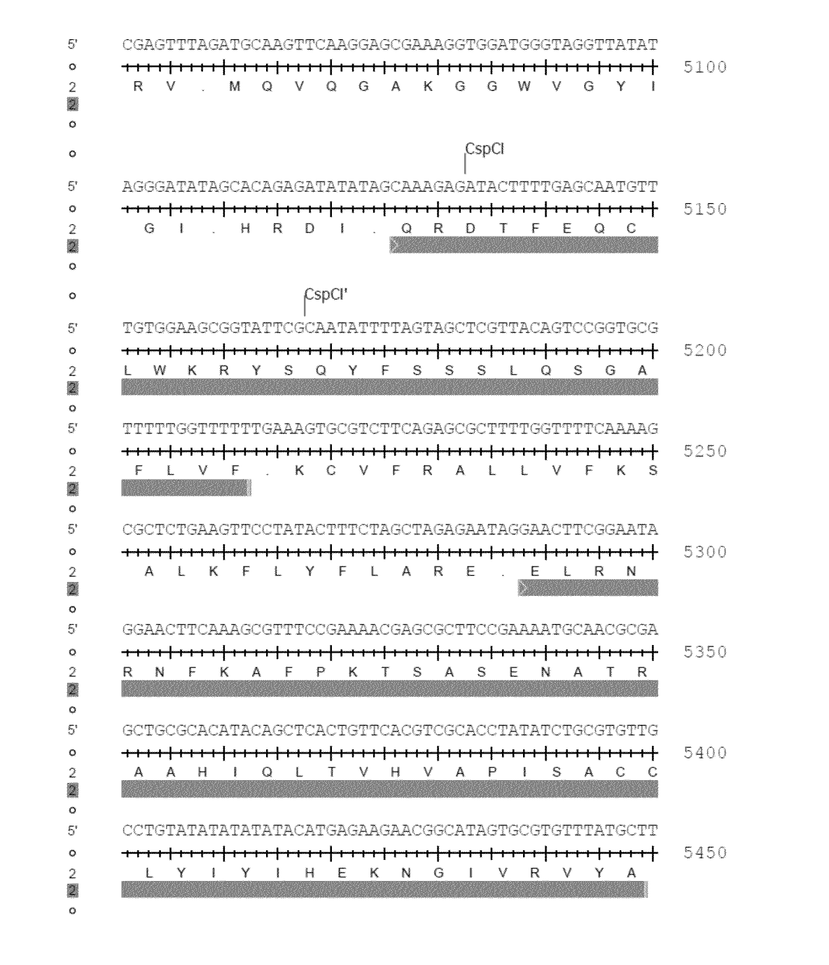

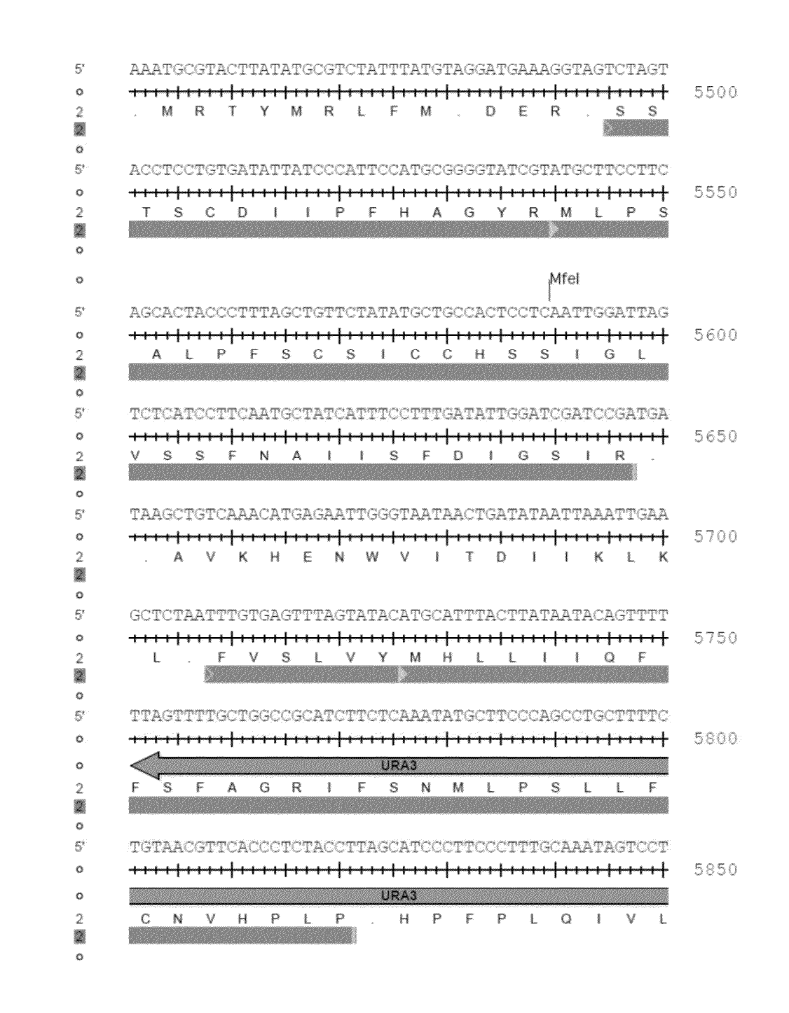

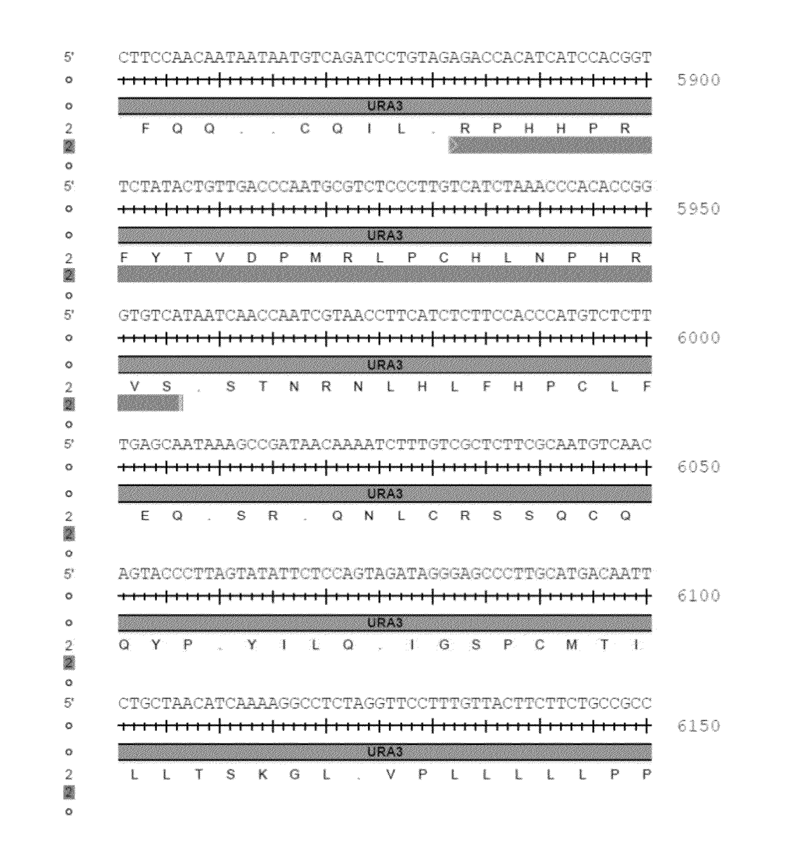

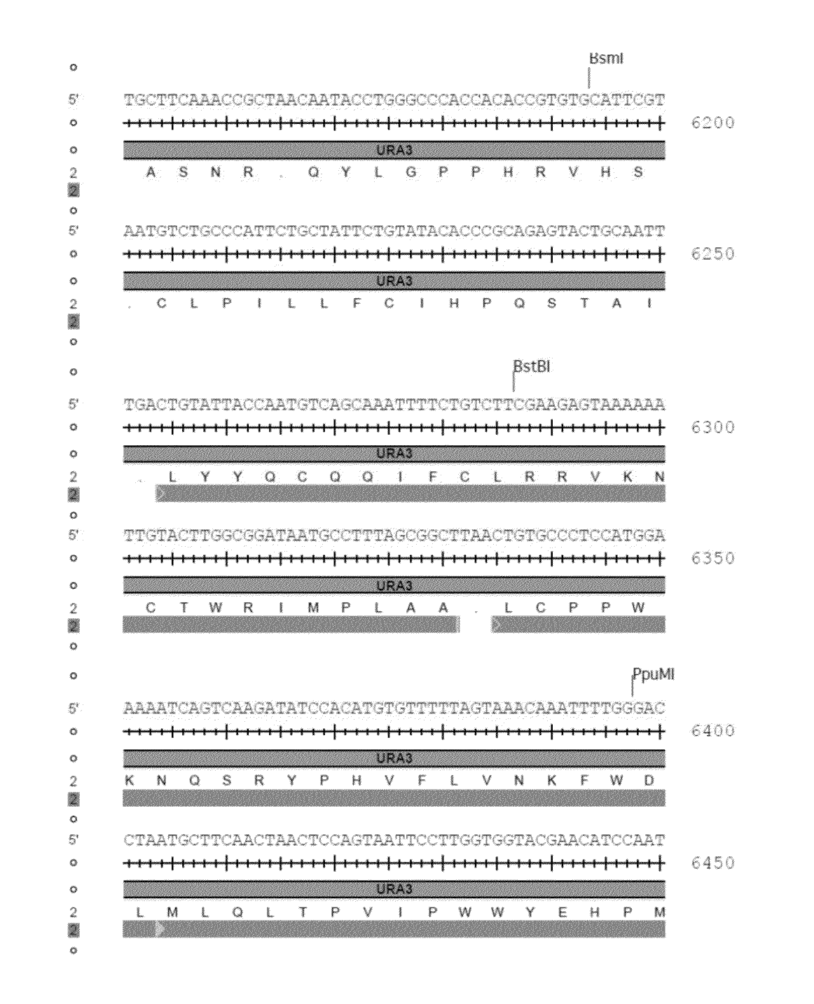

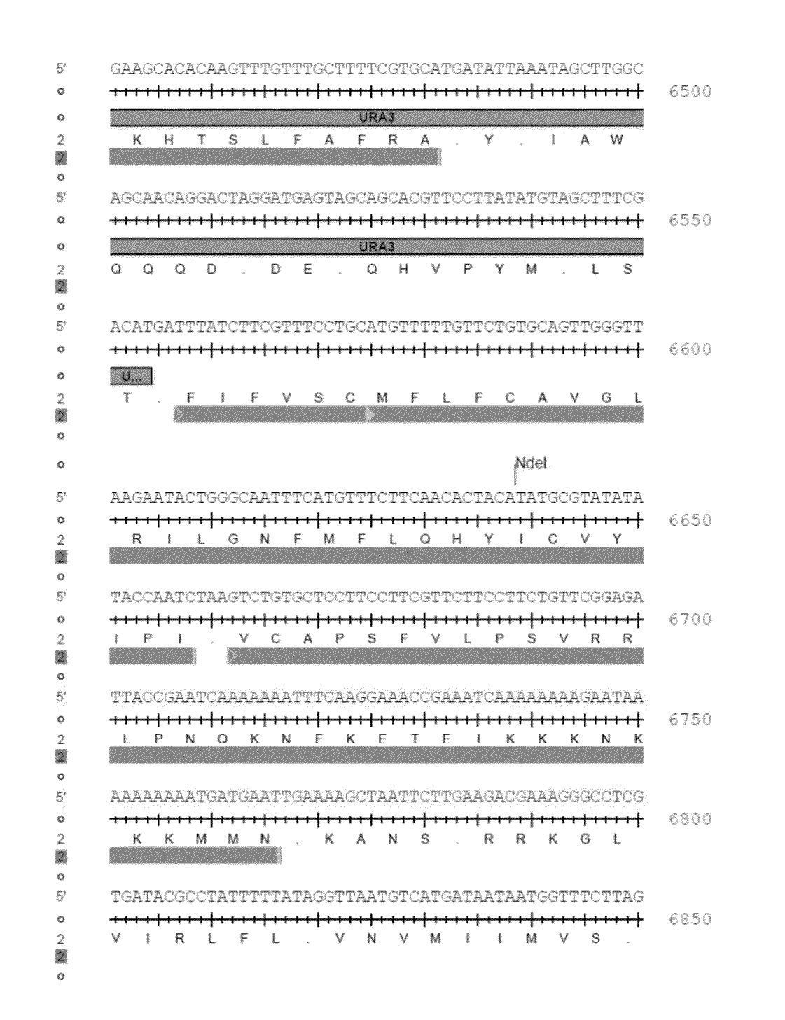

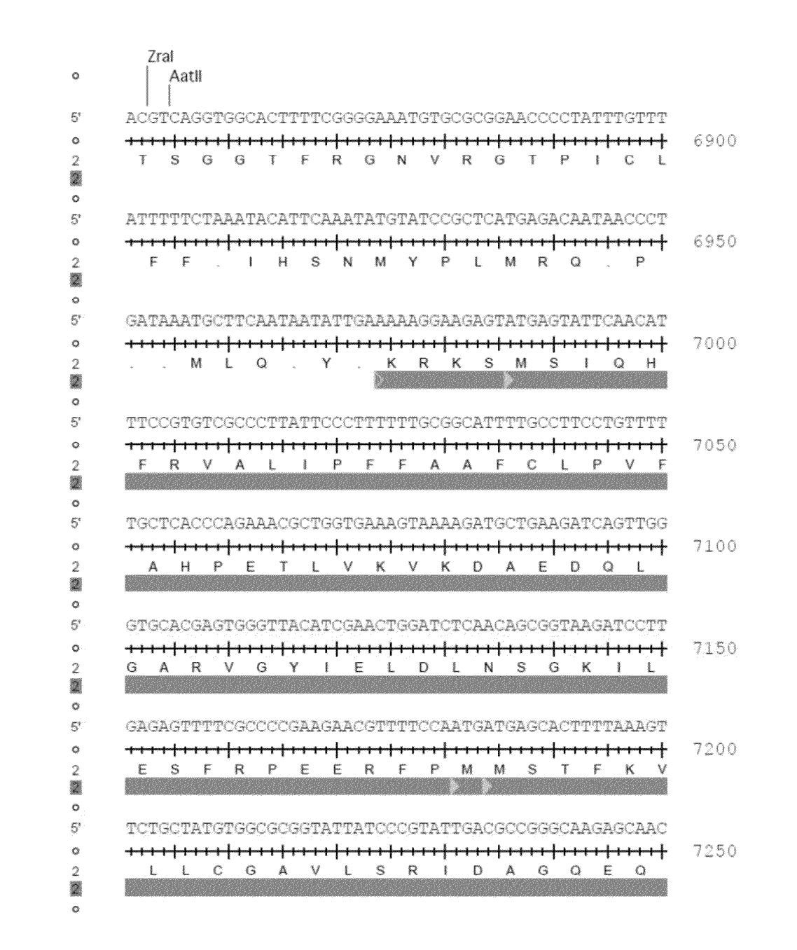

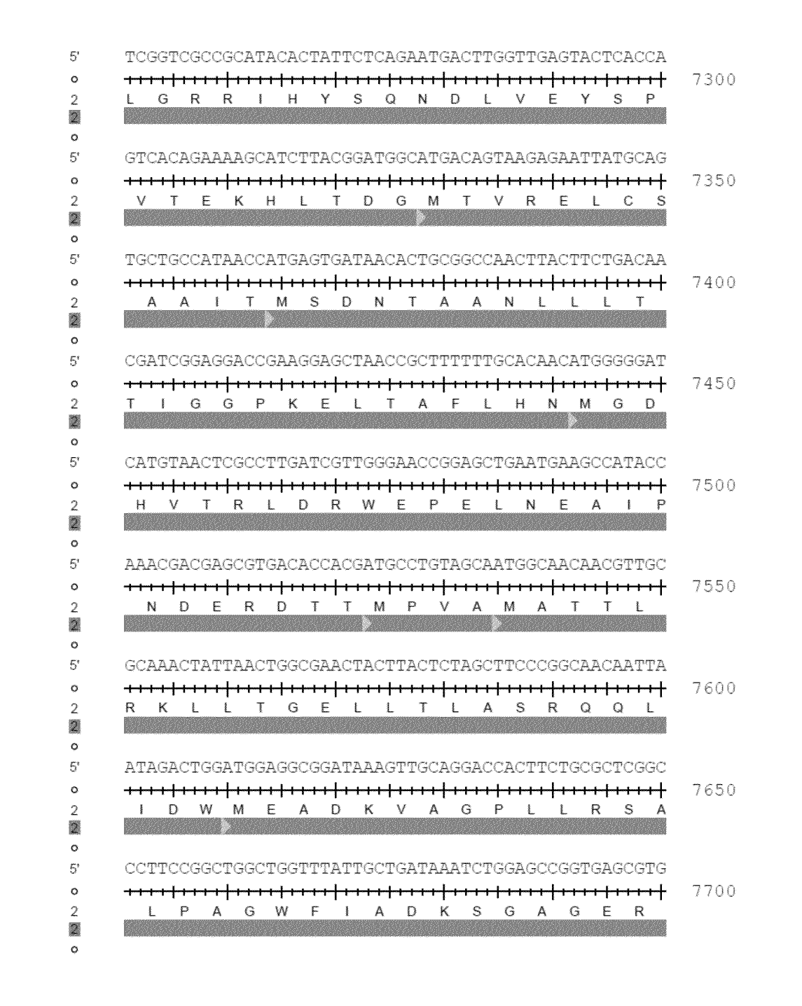

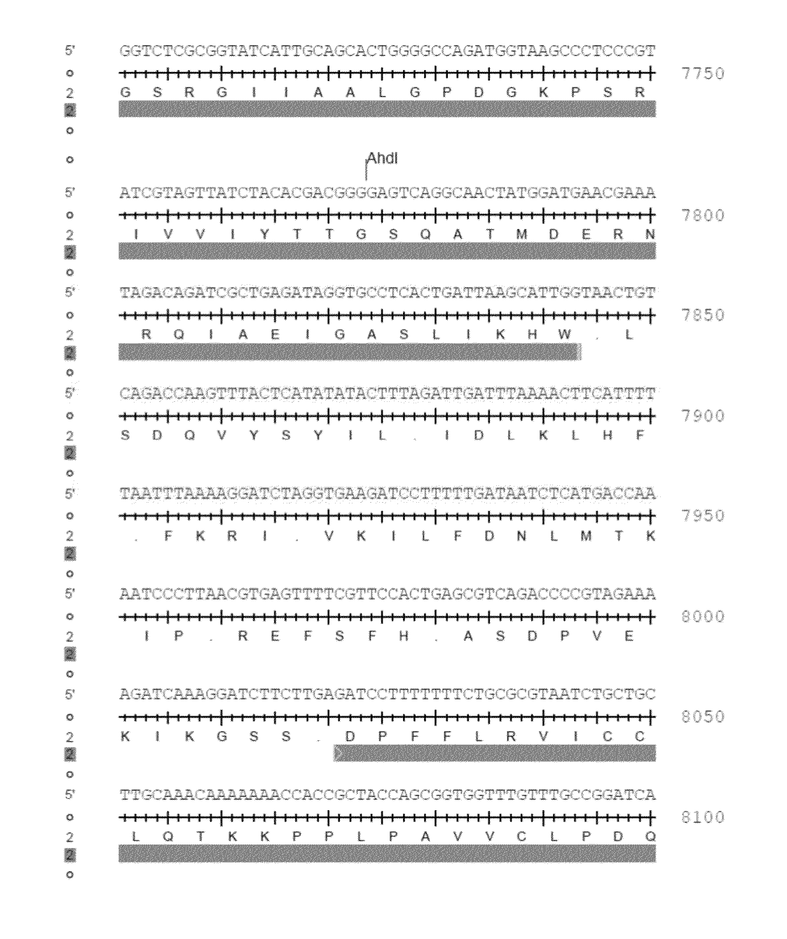

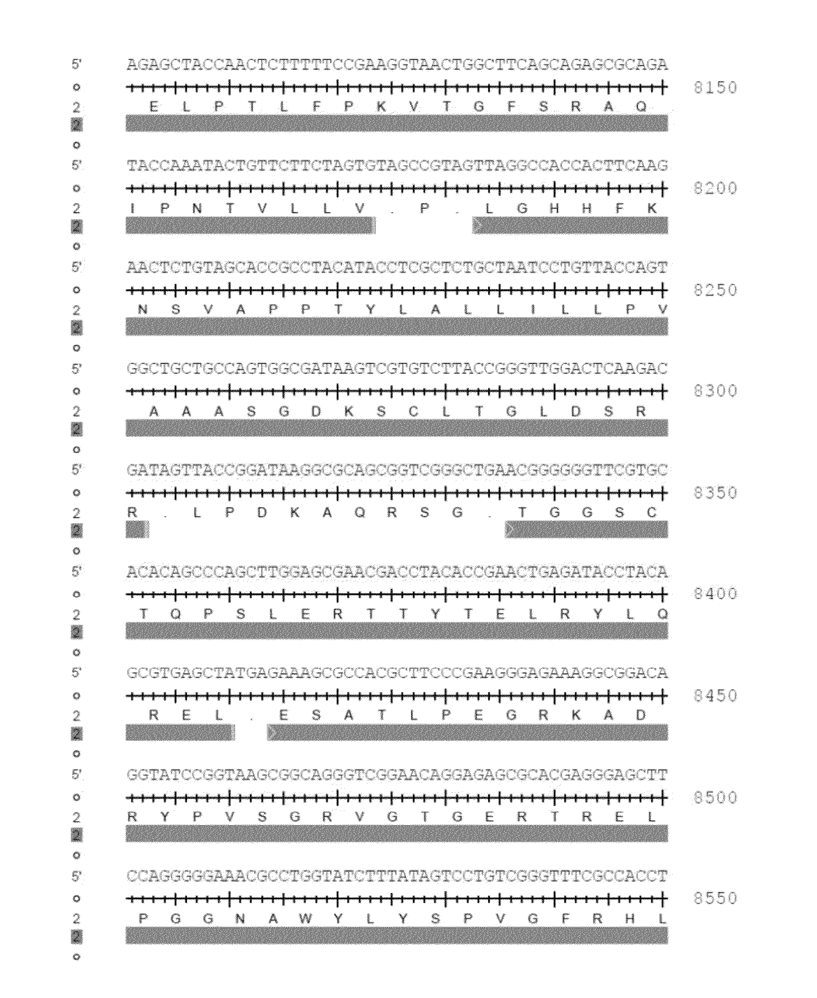

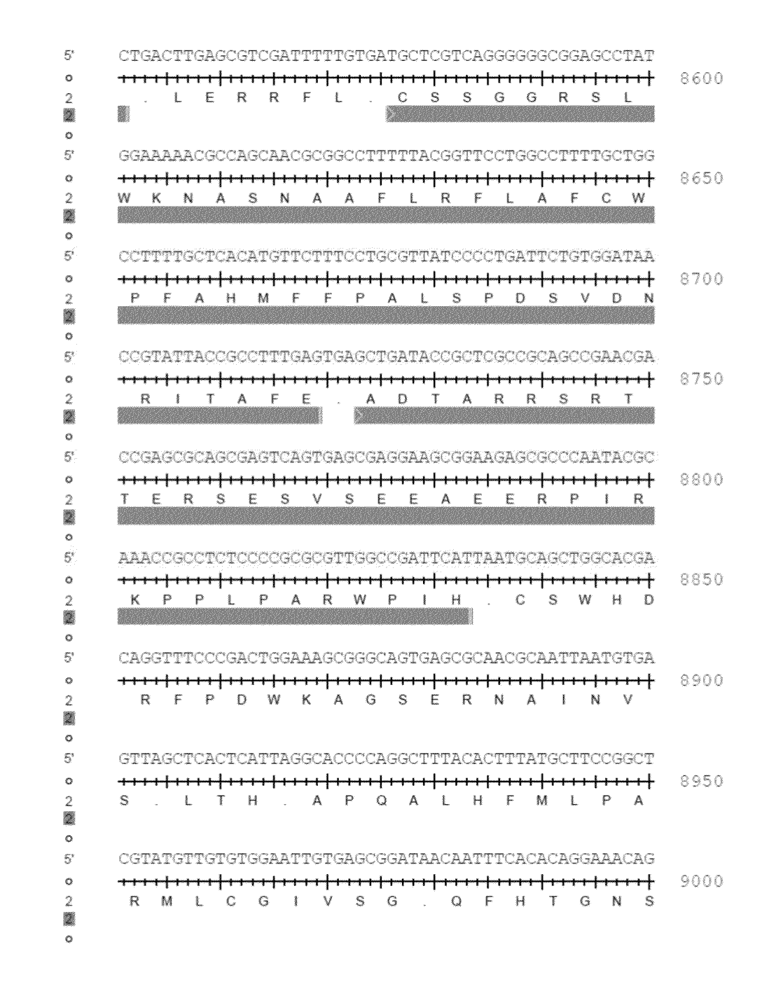

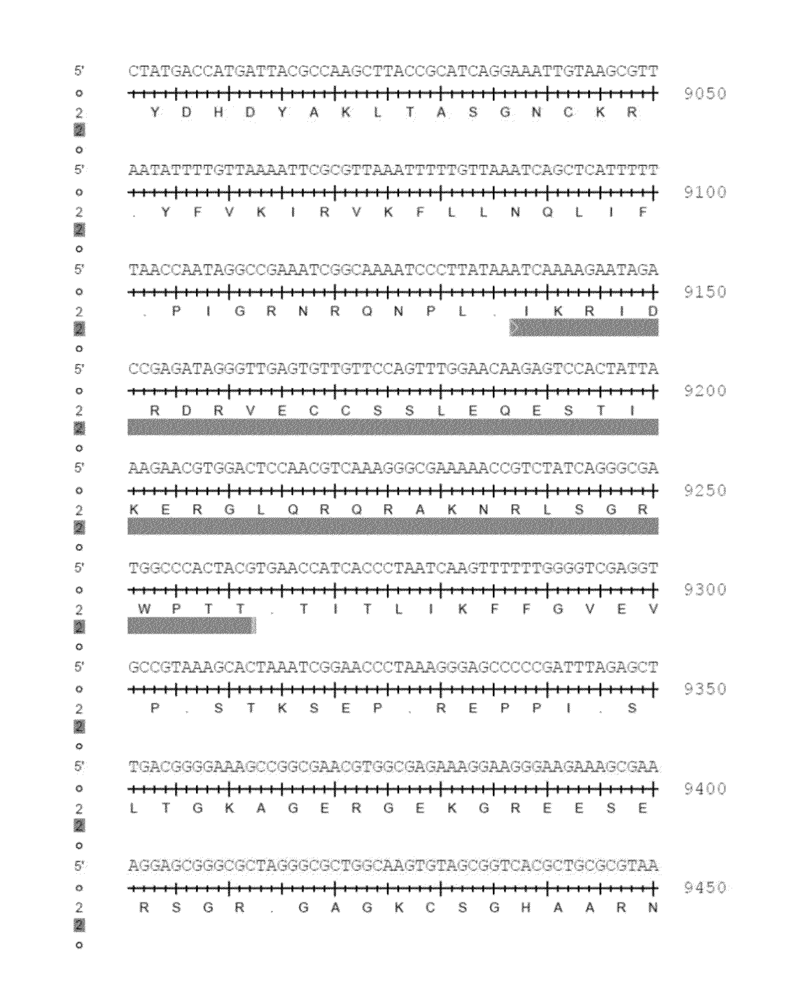

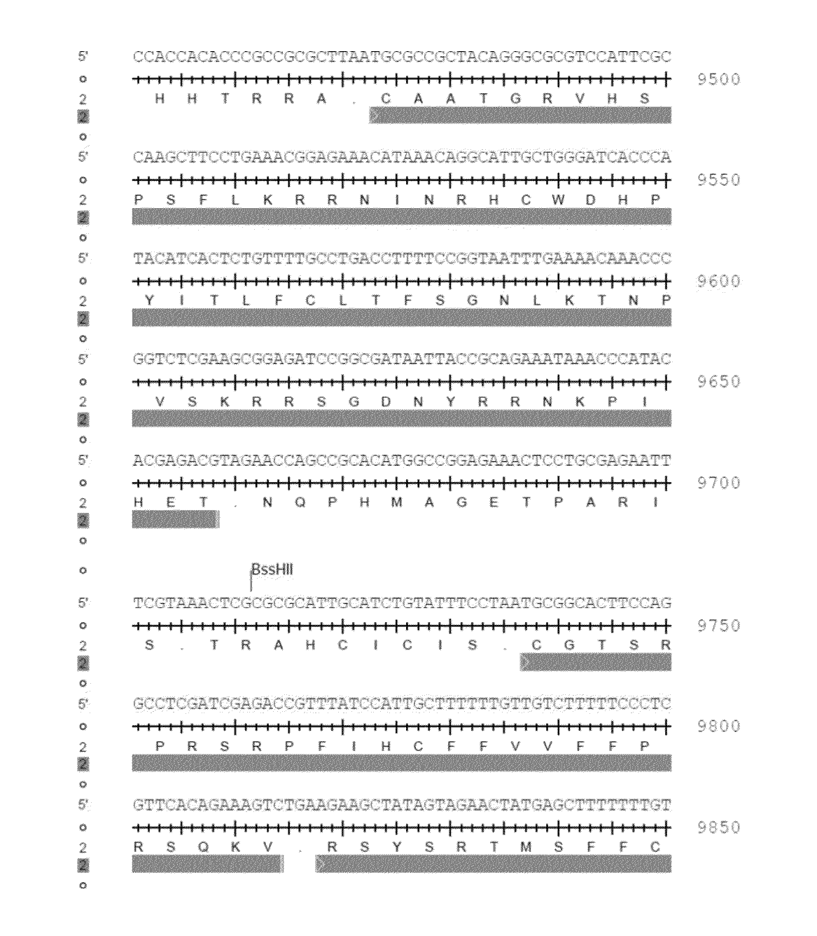

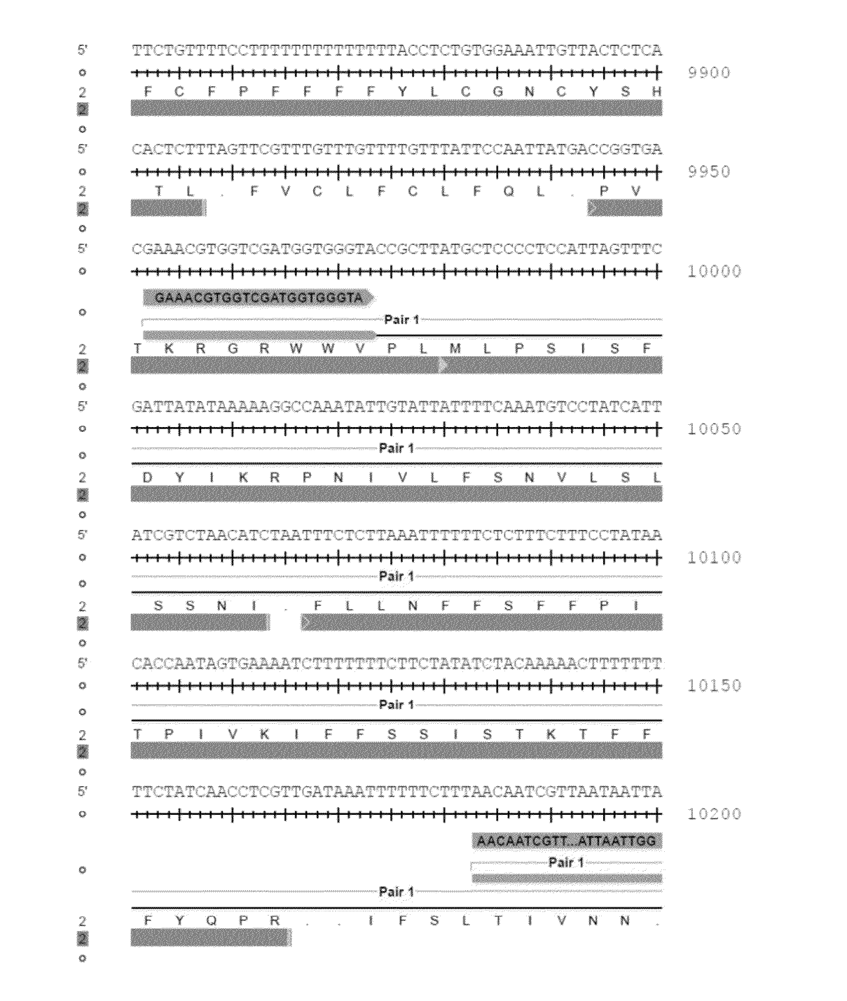

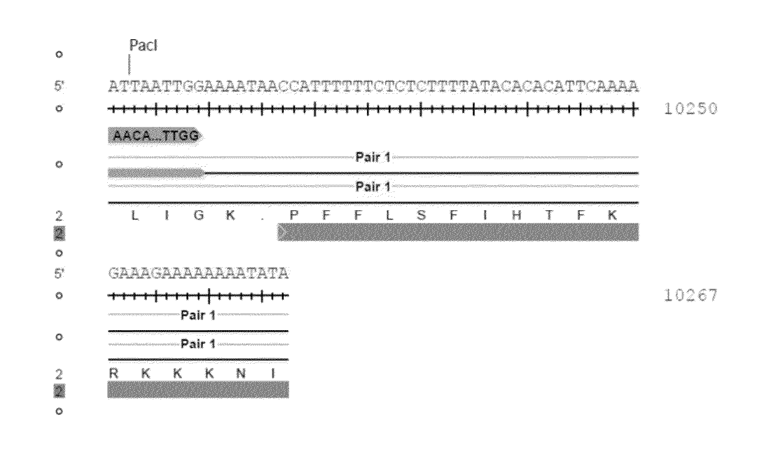

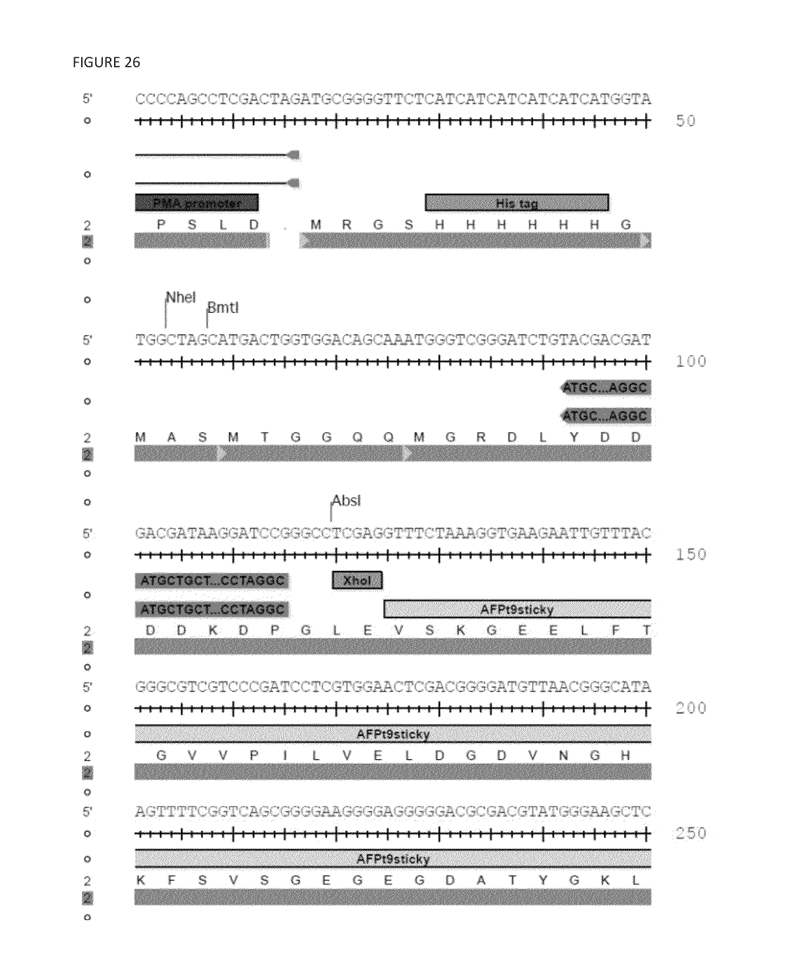

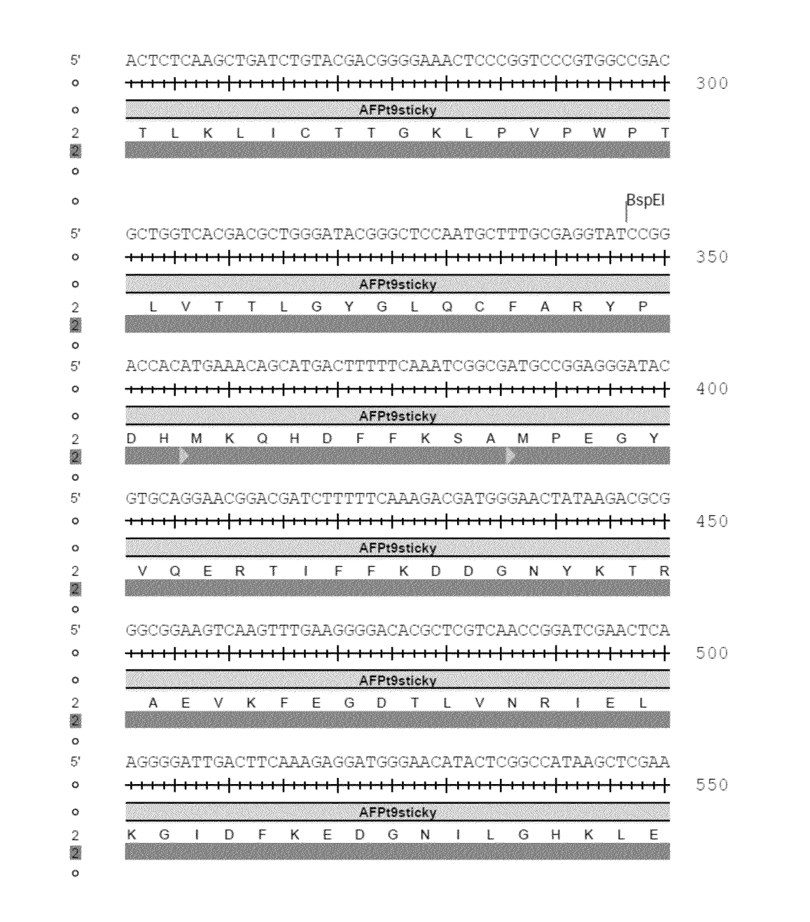

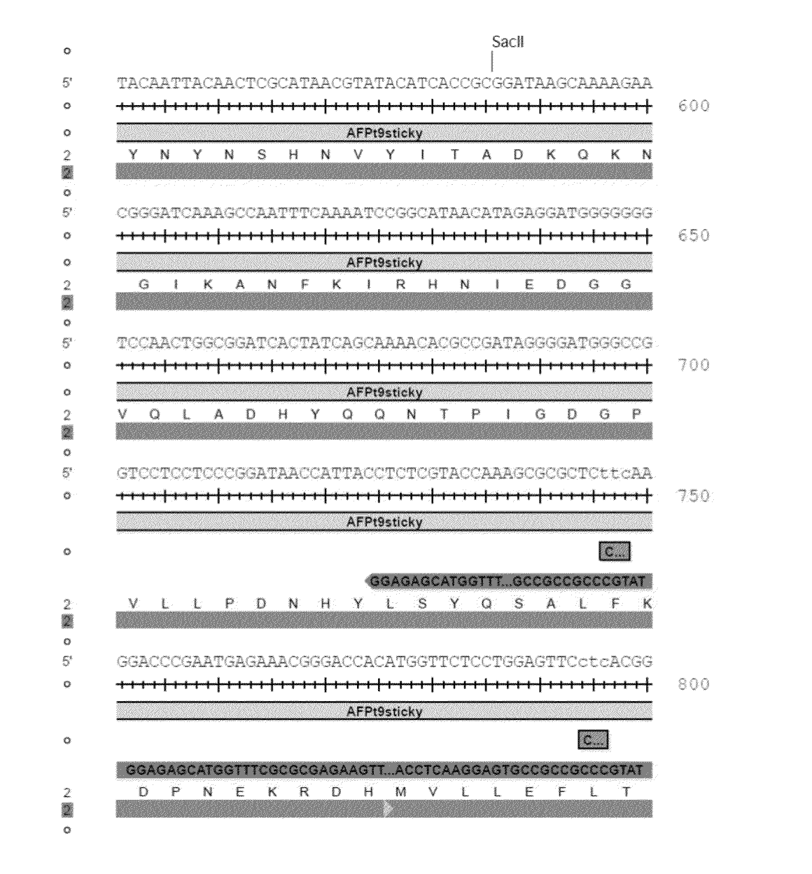

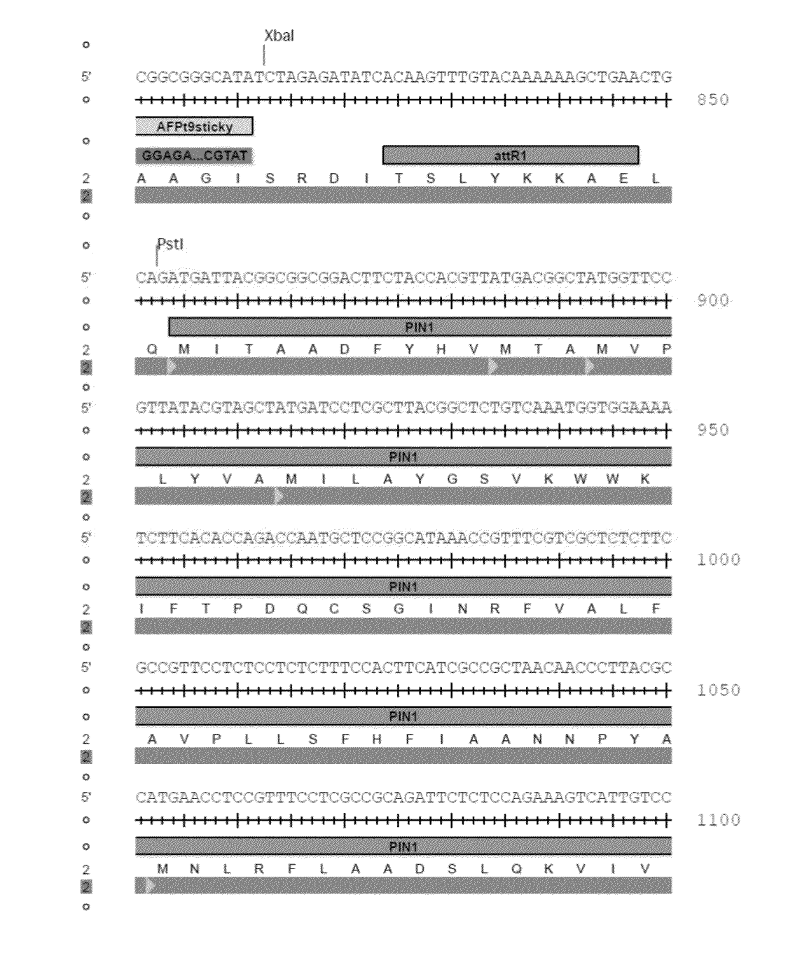

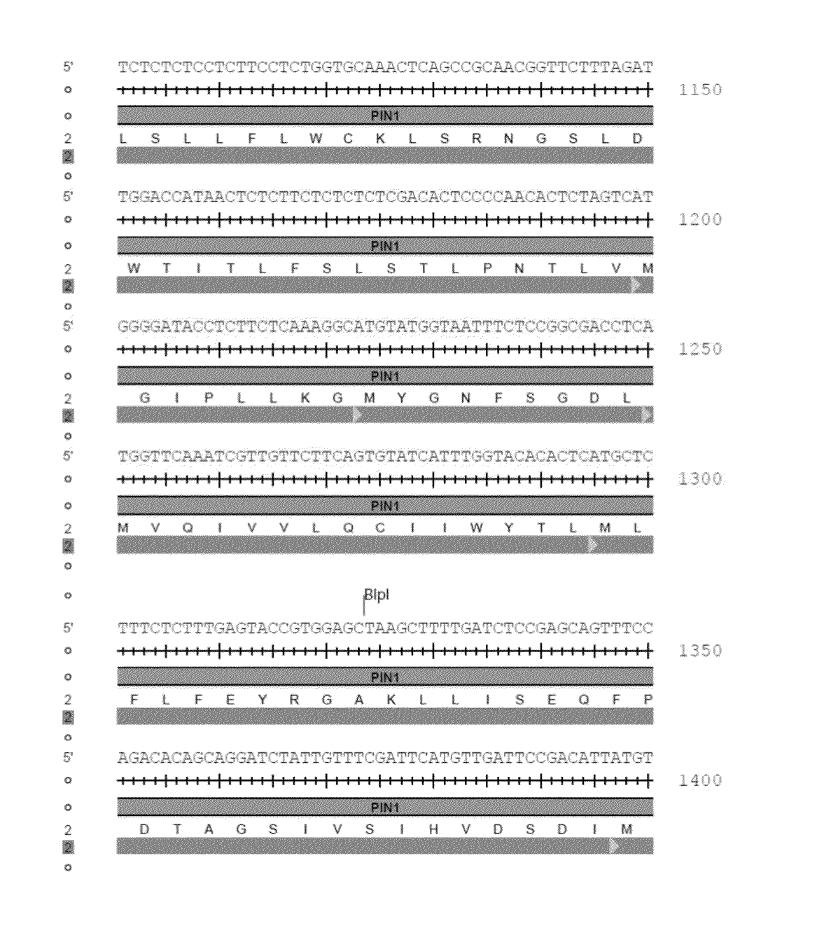

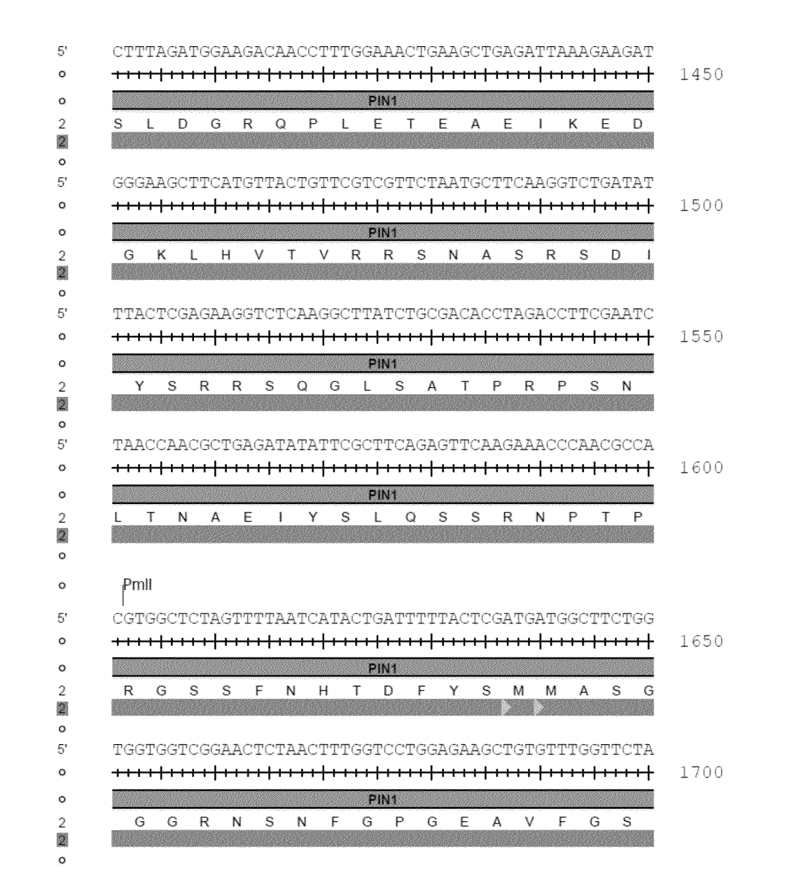

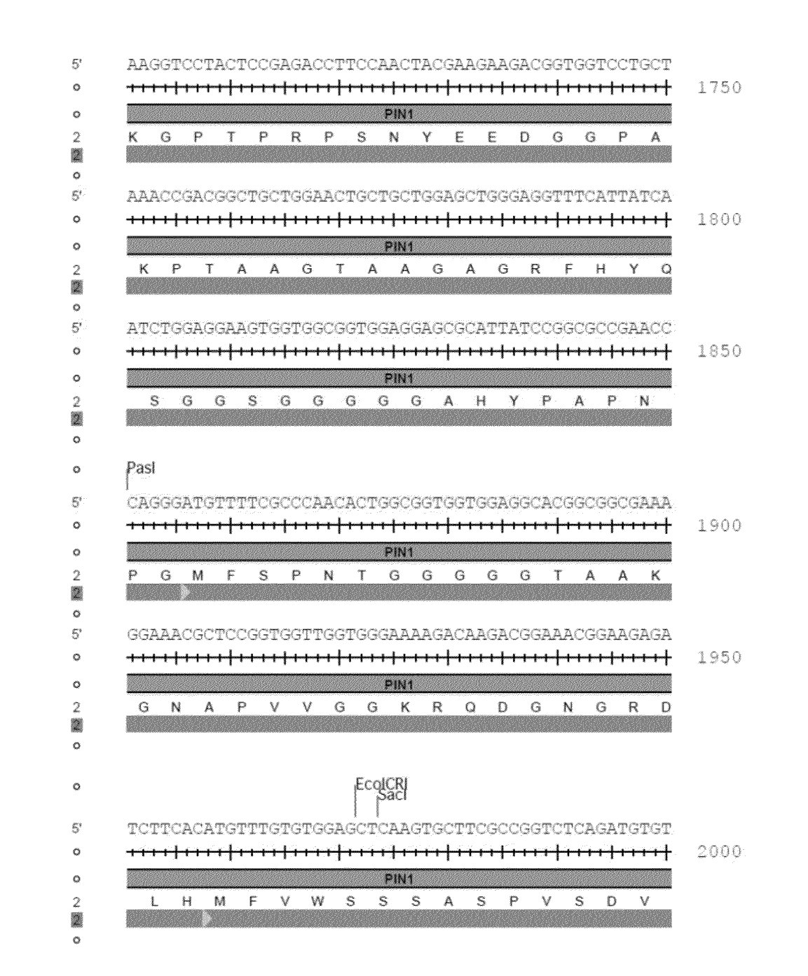

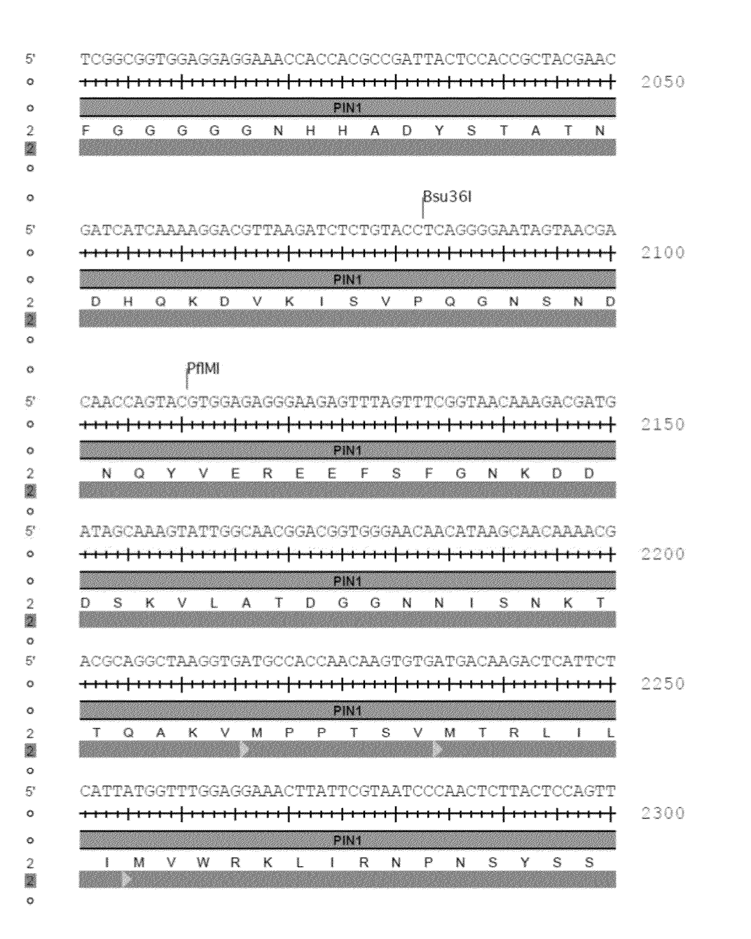

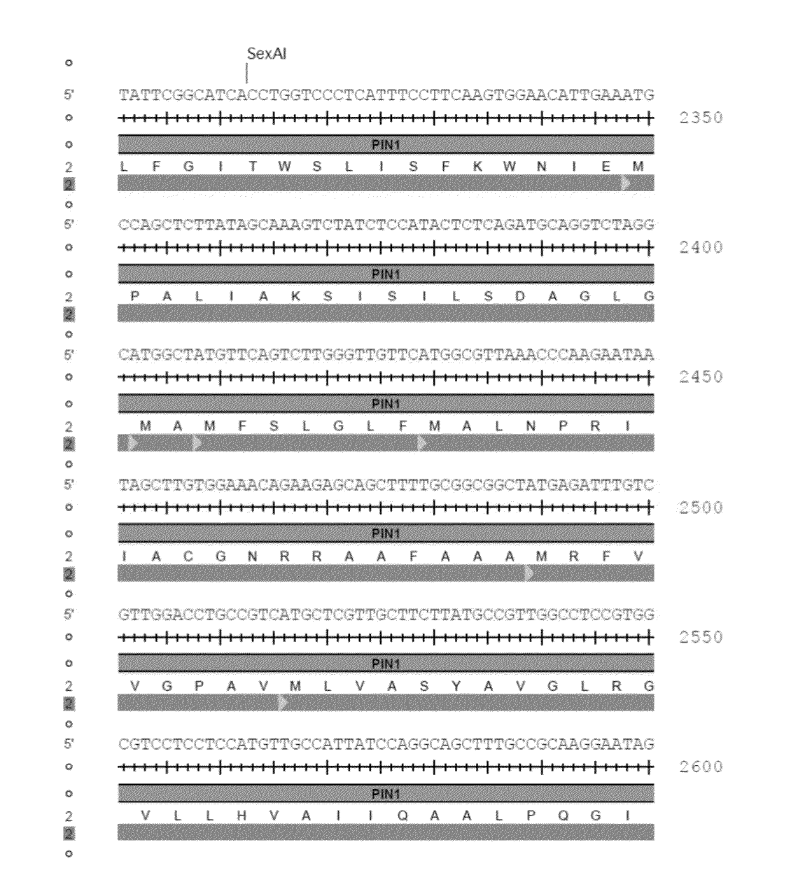

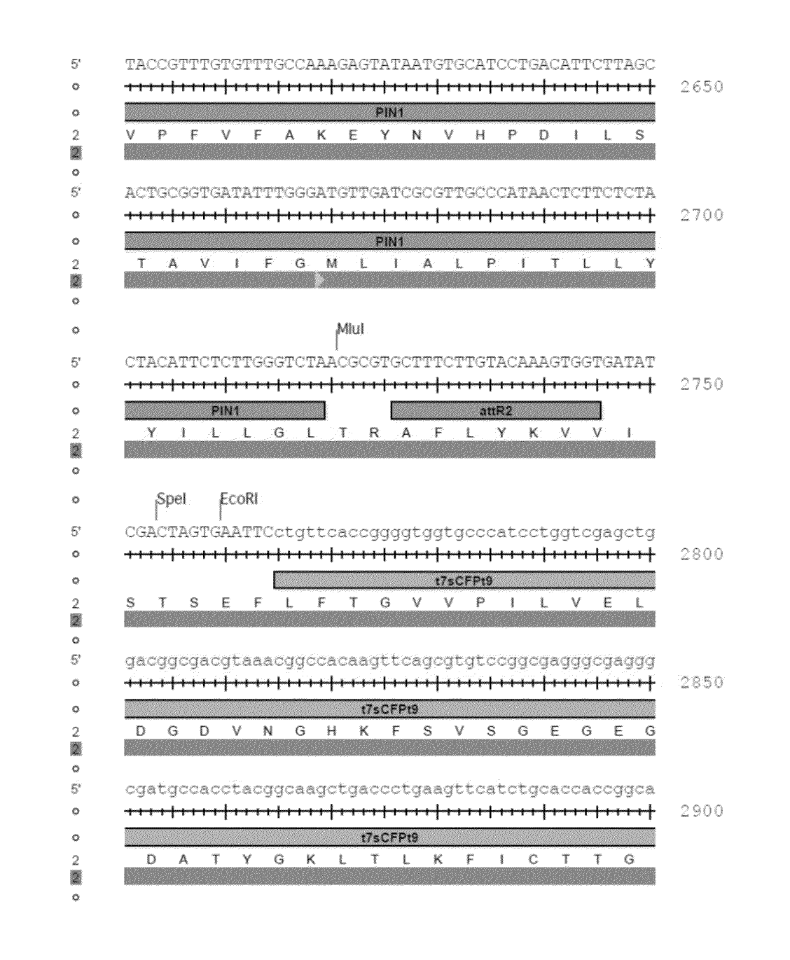

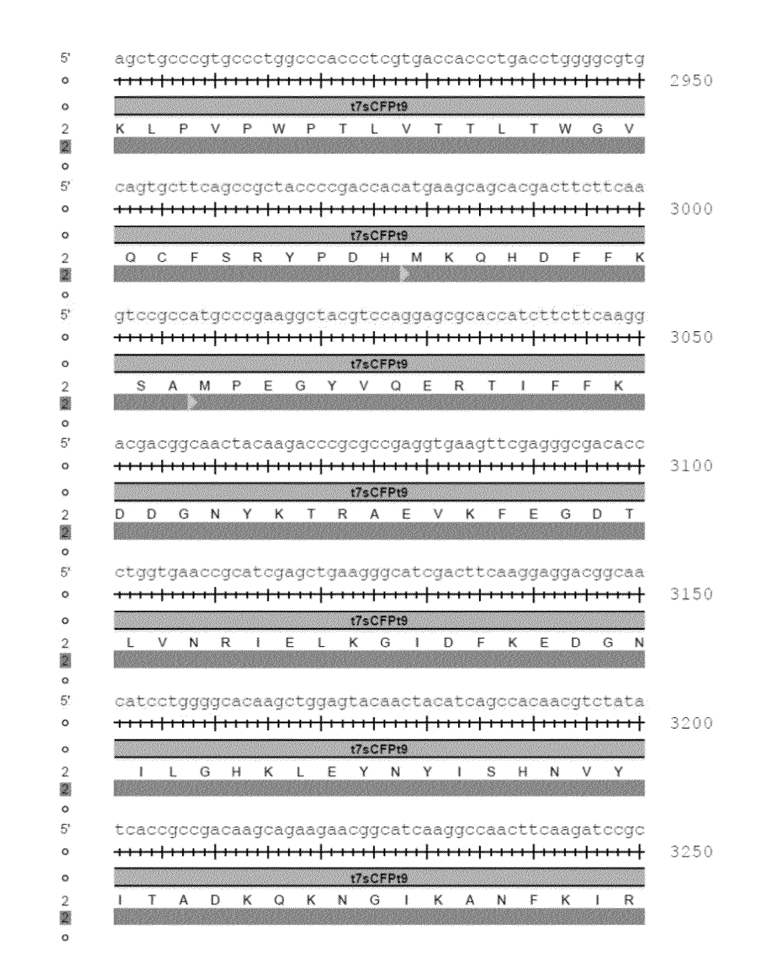

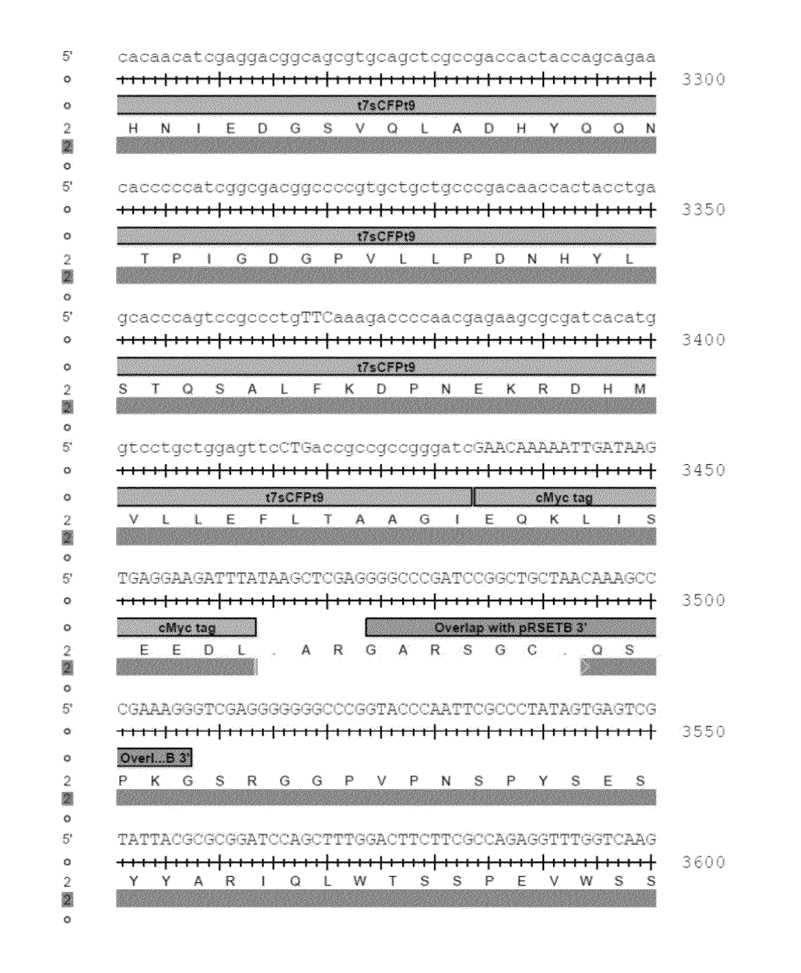

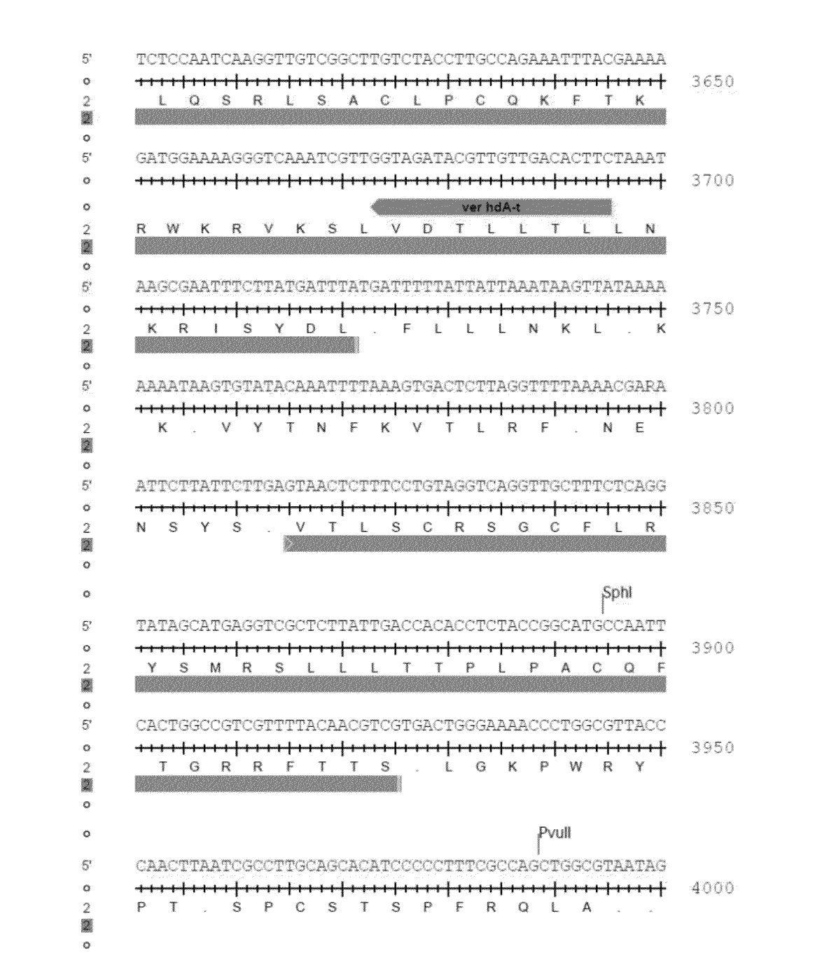

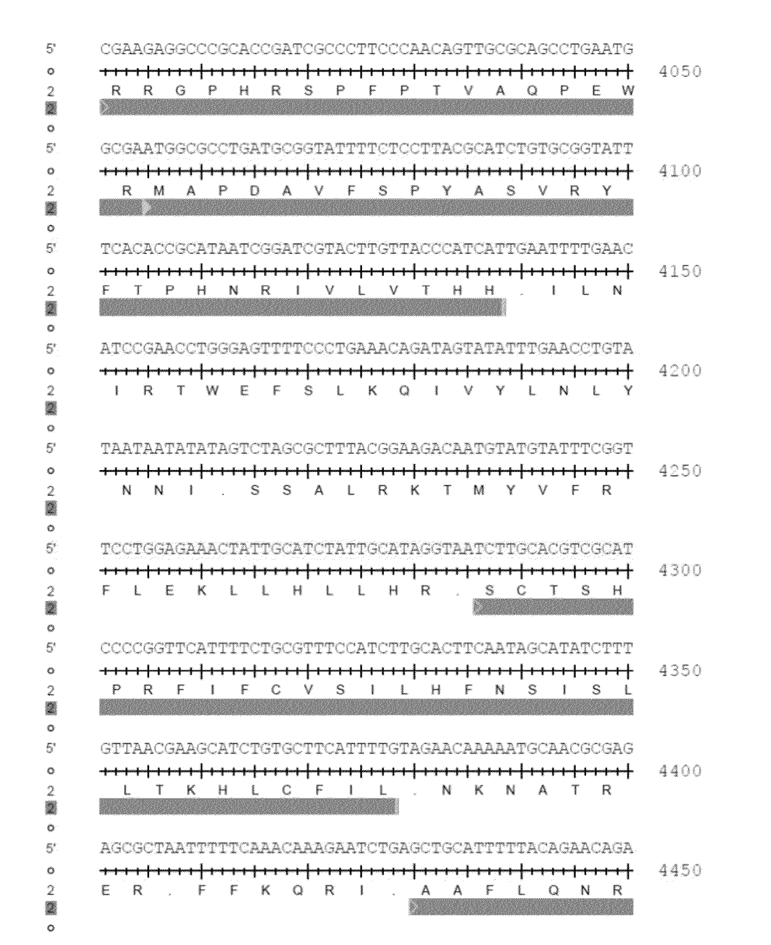

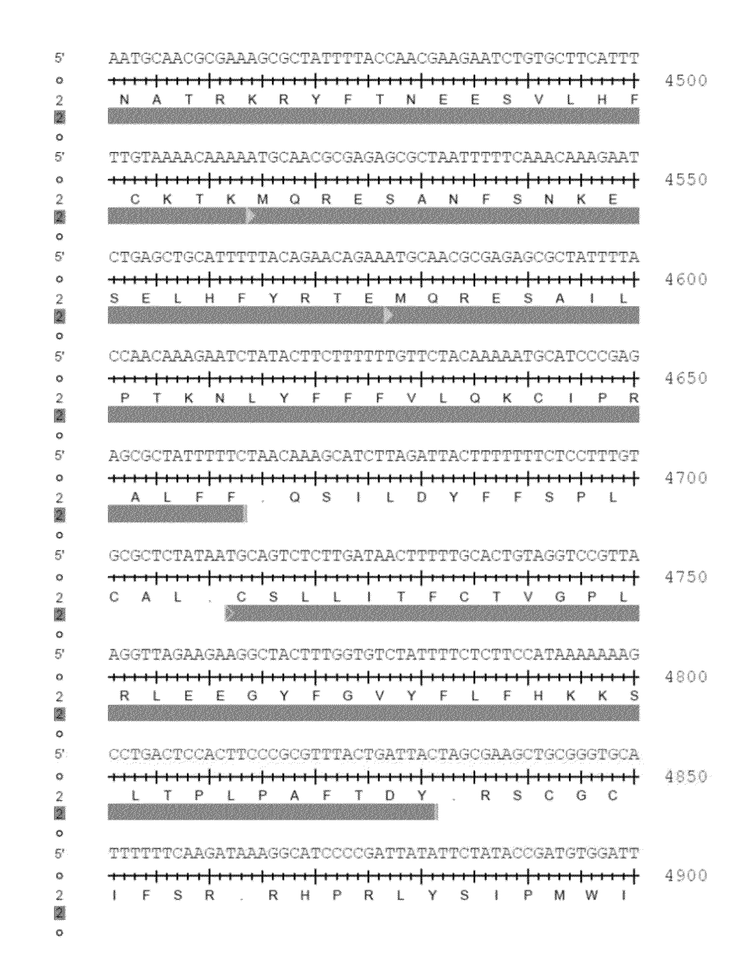

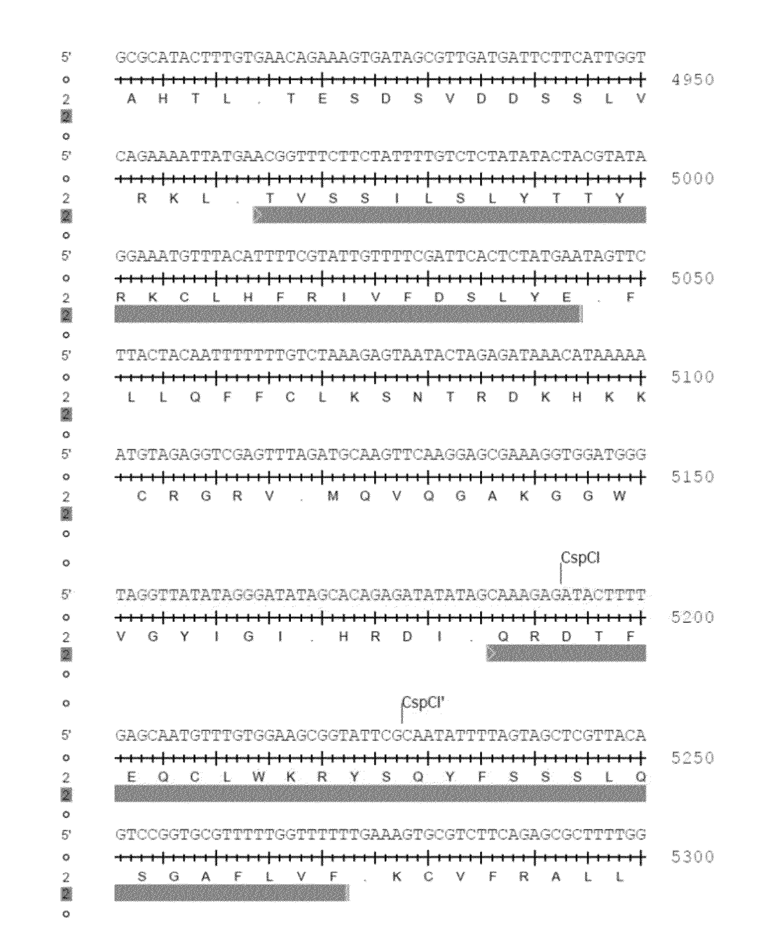

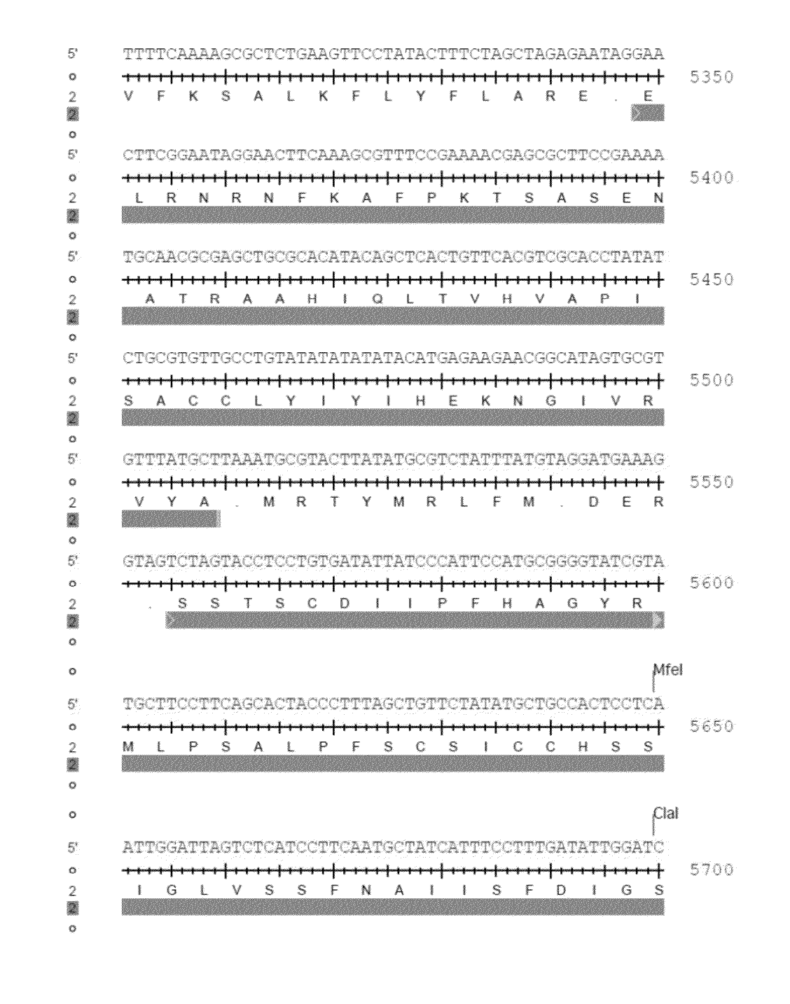

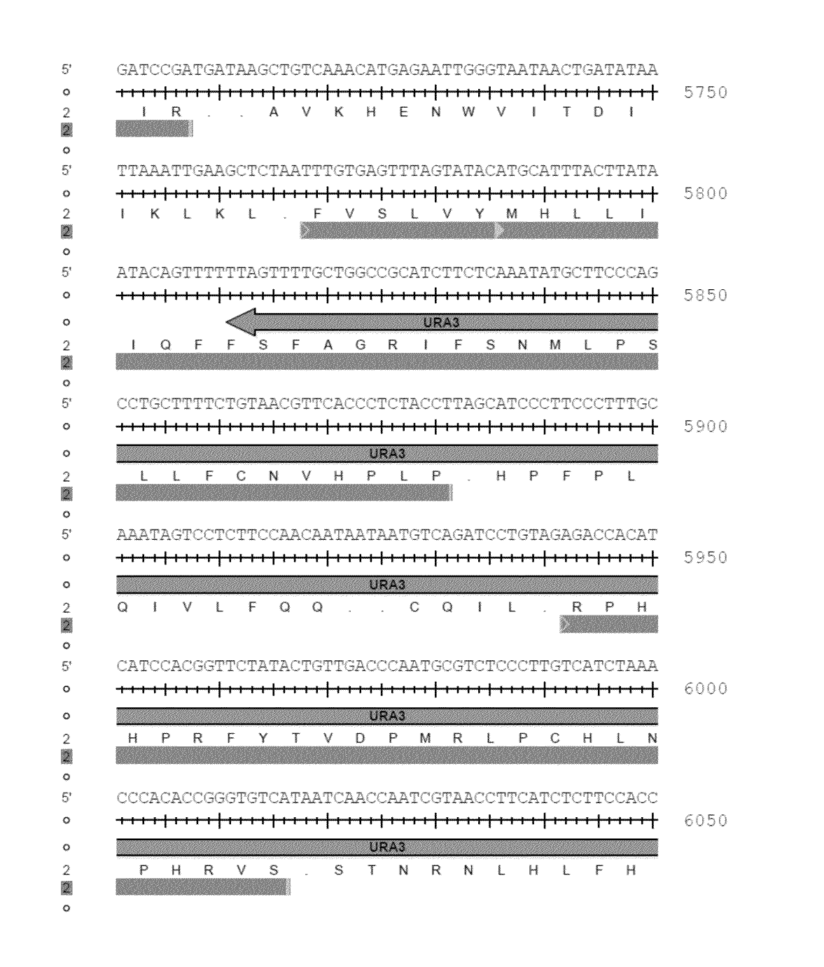

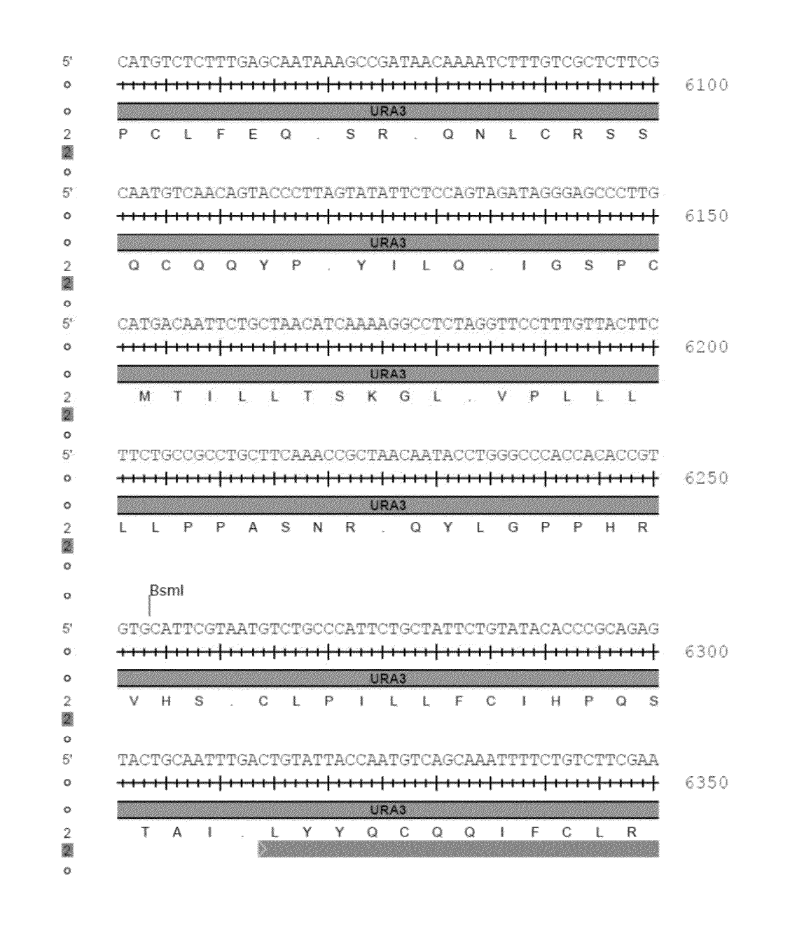

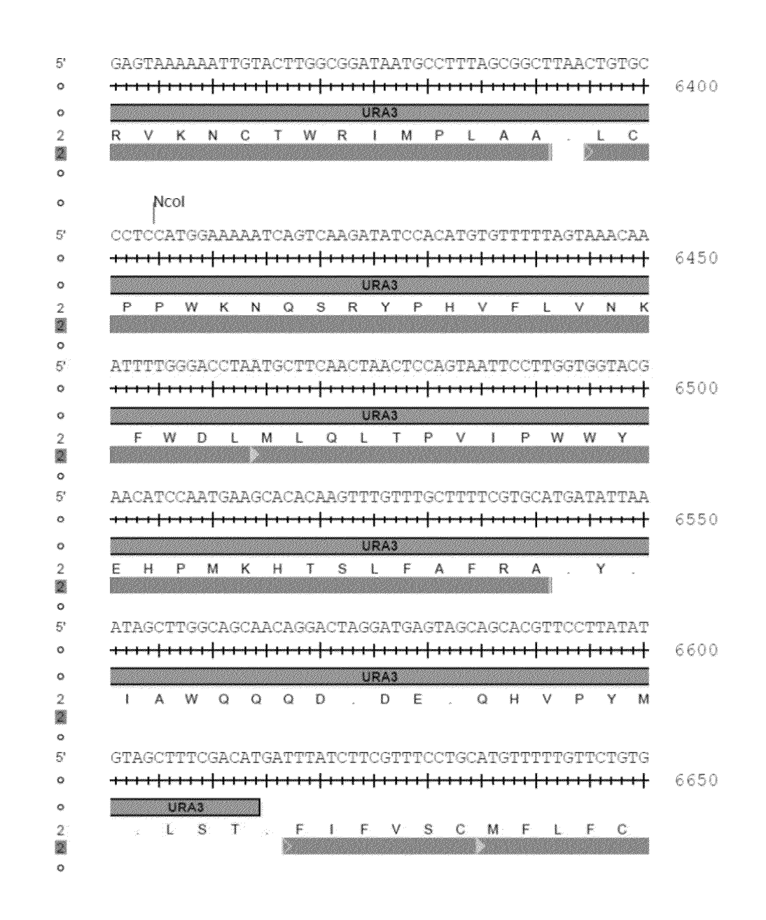

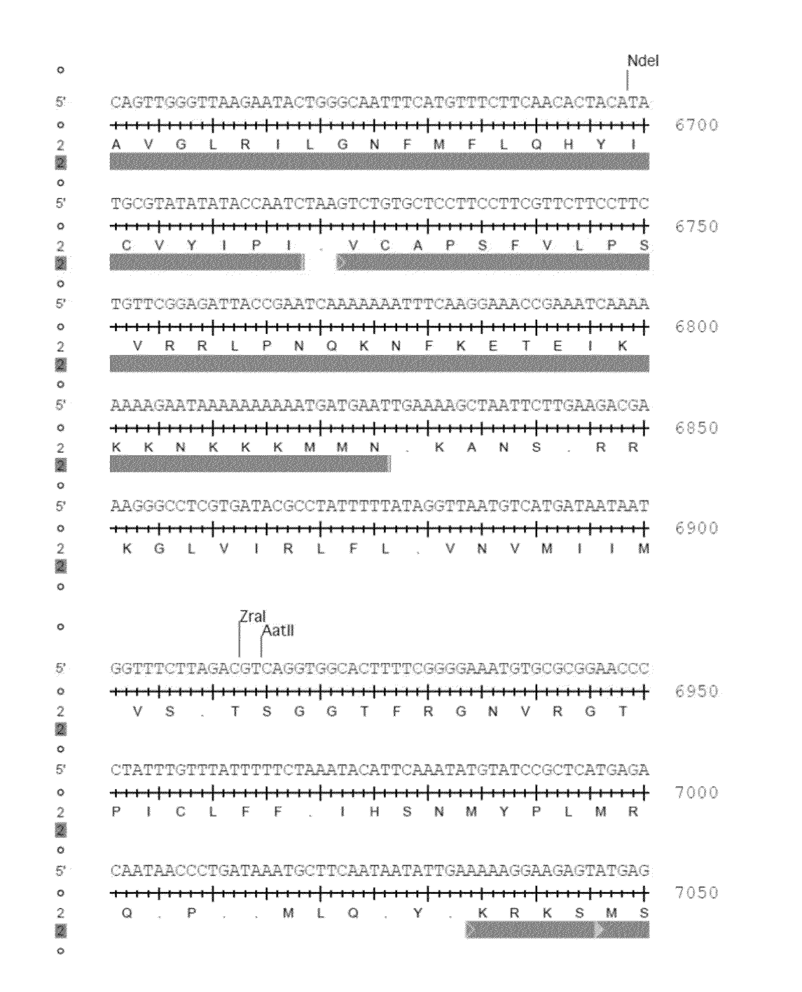

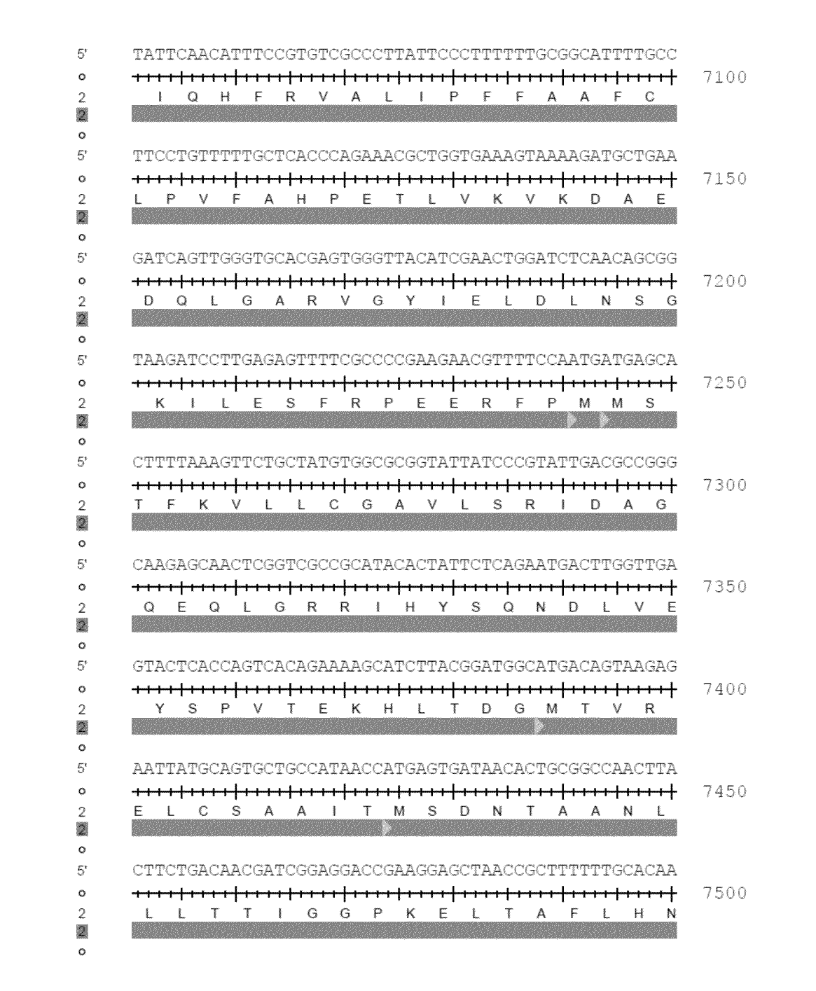

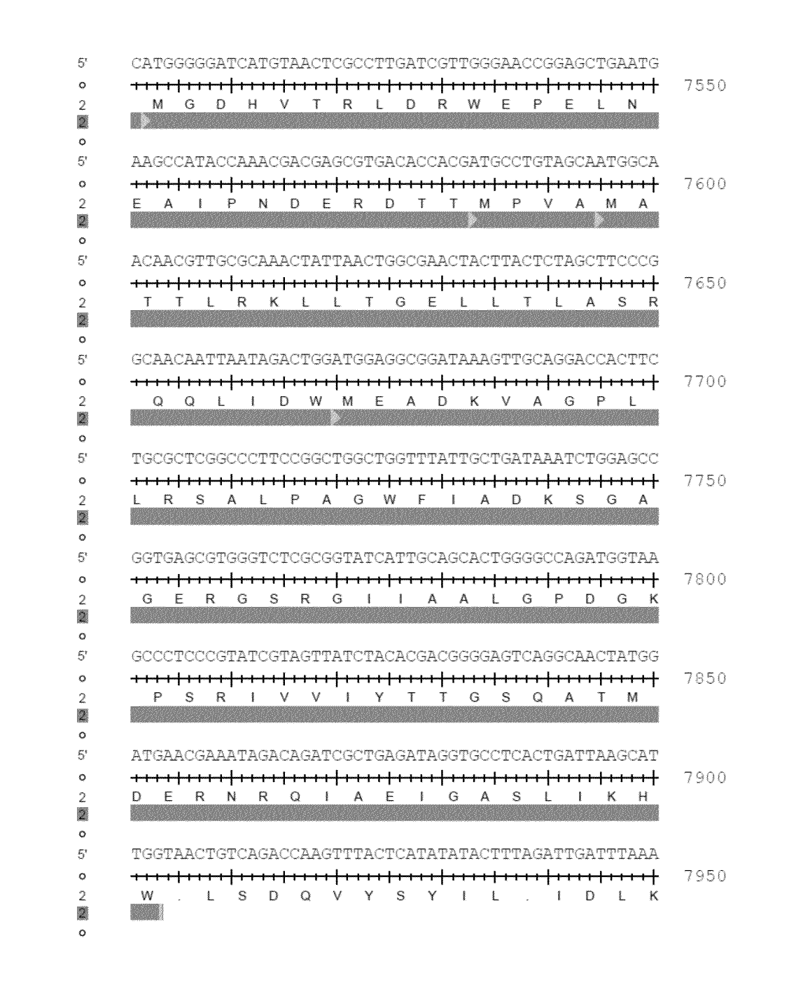

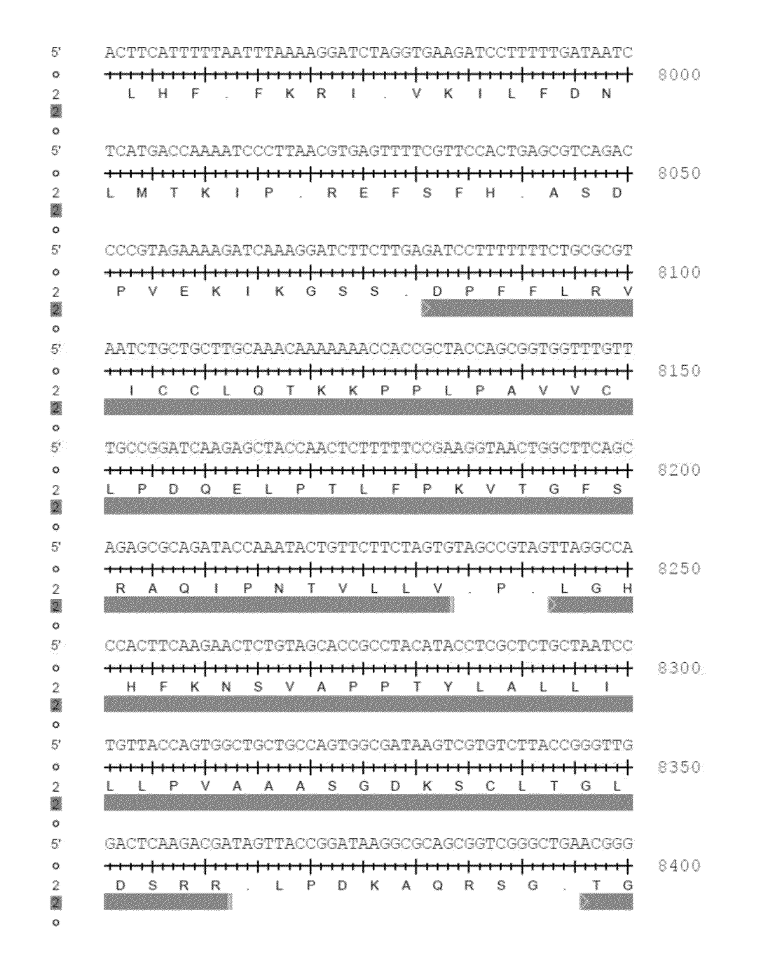

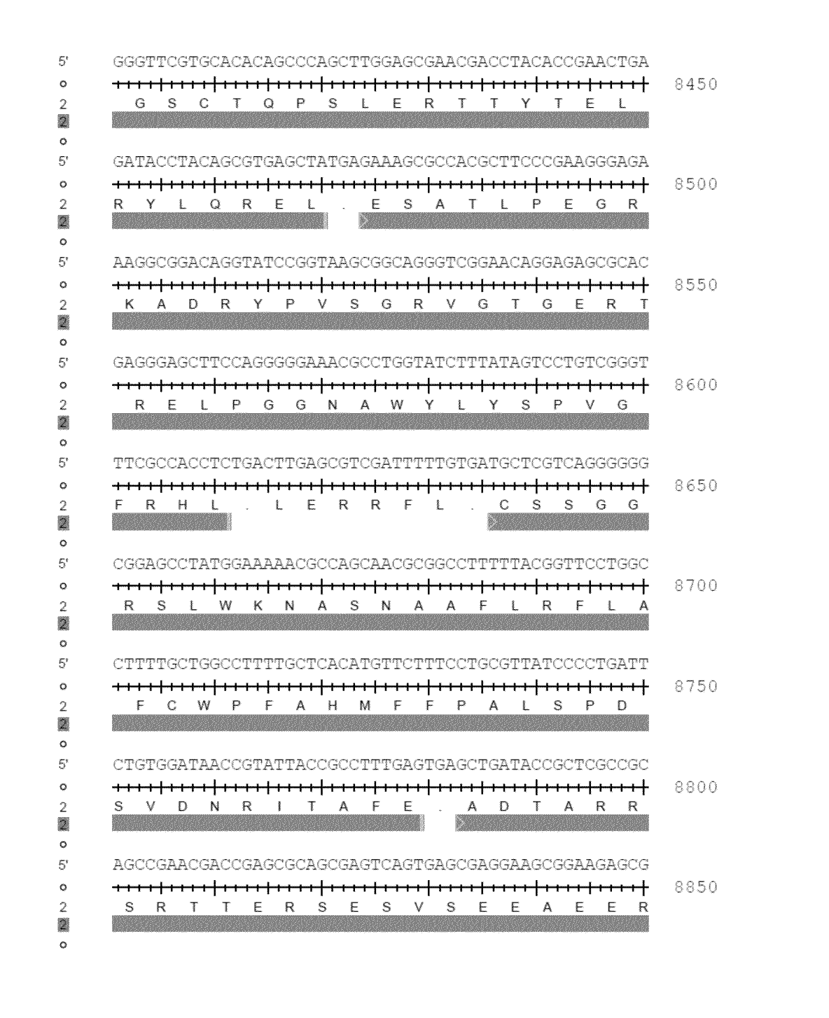

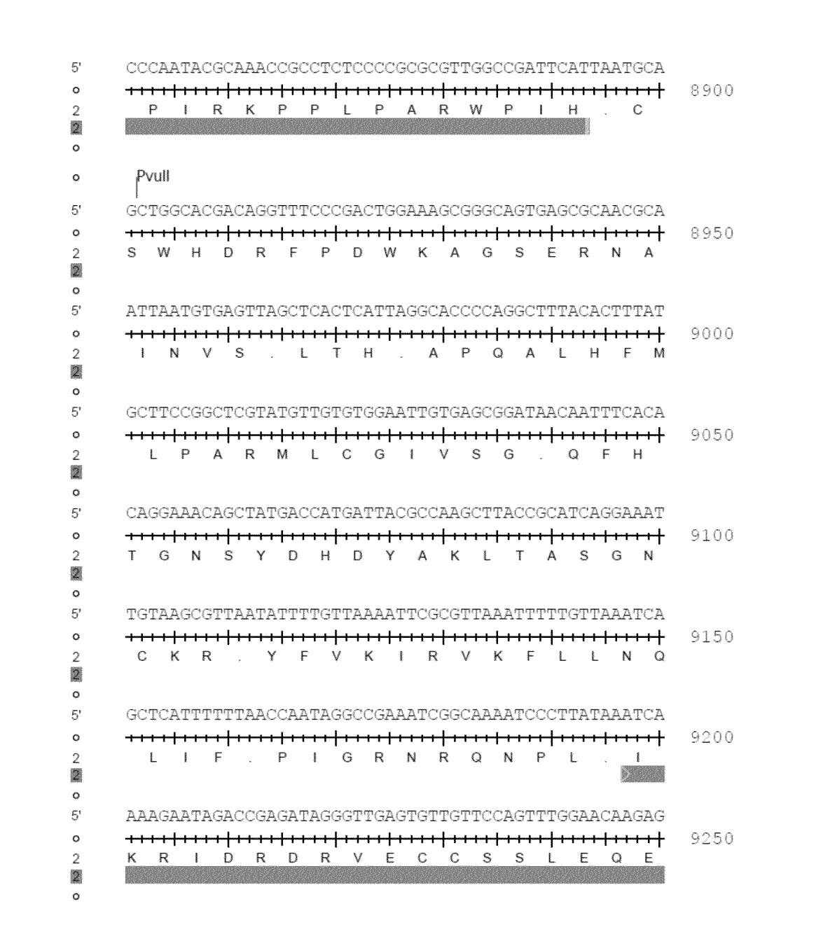

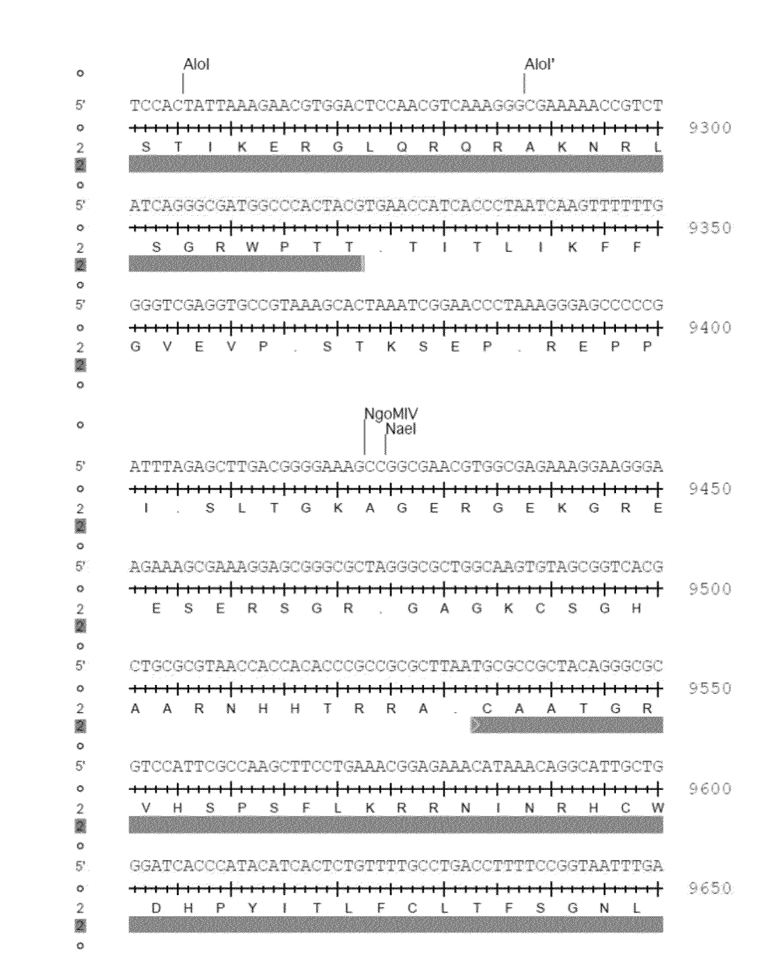

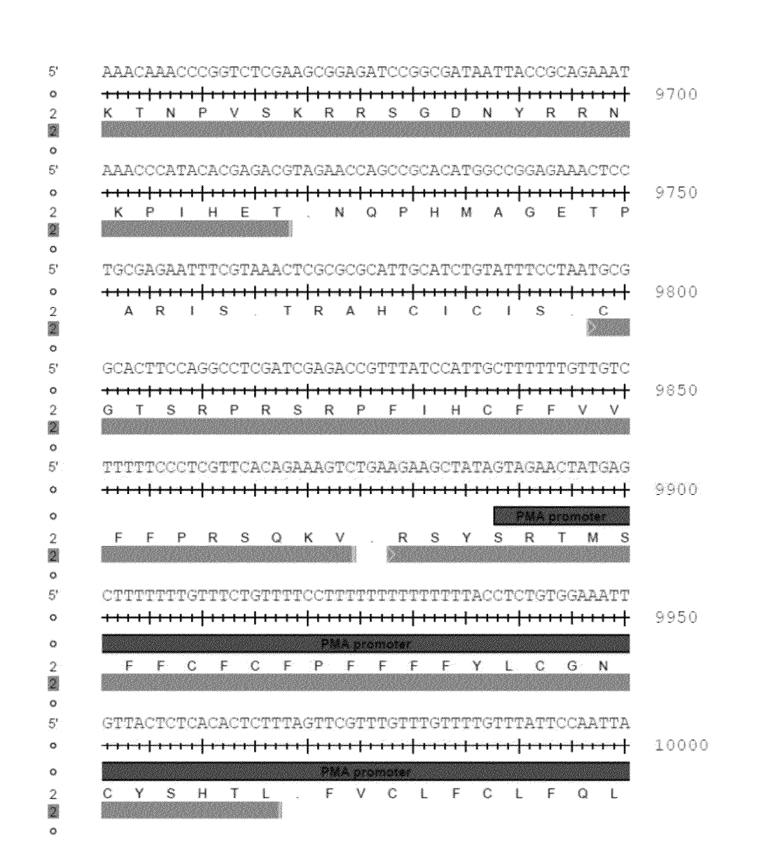

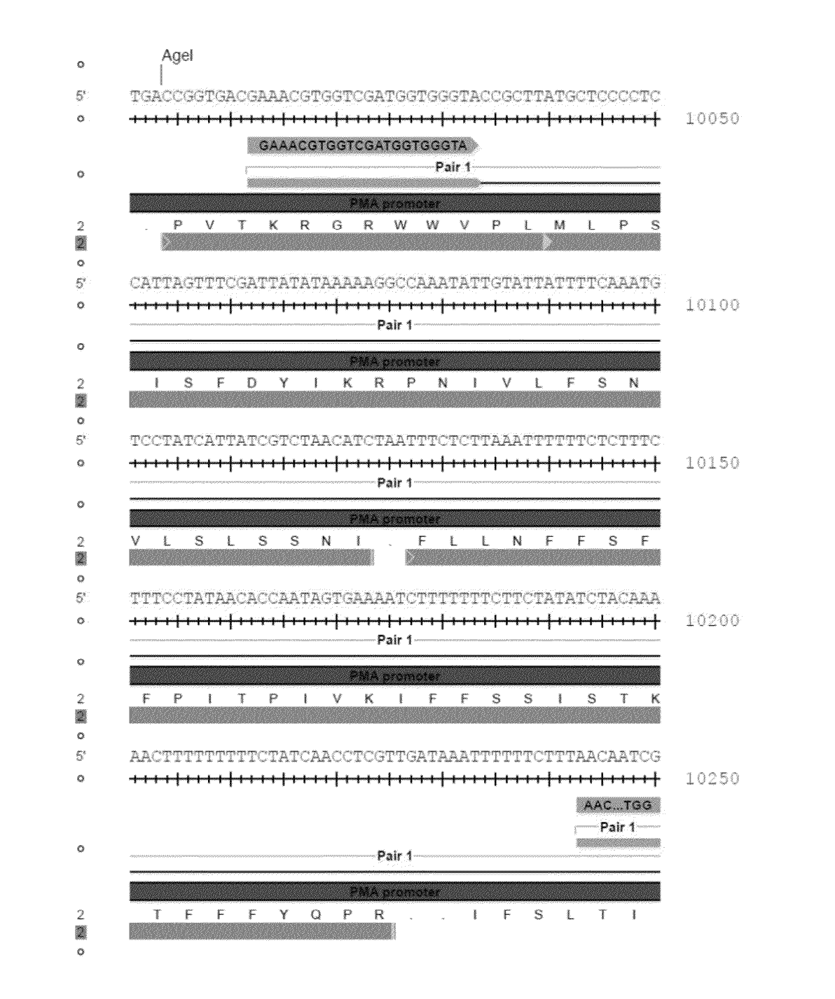

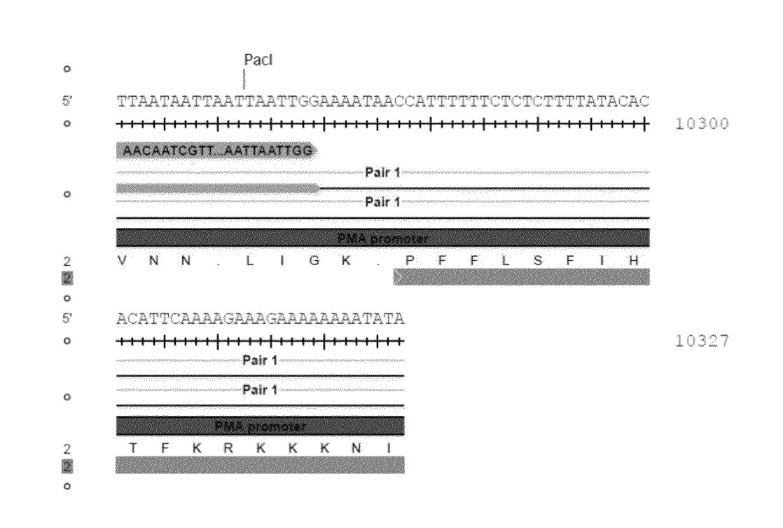

The OzTrac-MSL10 FRET sensor can detect a range of osmolarity concentration changes. Upon treatment of different concentrations of NaCl and other osmolytes, concentration-dependent FRET changes were detected, which can be fitted to a Hill curve. See FIG. 22. The calculation of the dissociation constant is around 0.5 M for NaCl and KCl, and around 1M for glycerol and glycerol.

What is claimed is:

1. A fusion protein comprising at least one fluorescent protein that is linked to at least one transporter protein comprising an N-terminus and a C-terminus, wherein the transporter protein changes three-dimensional conformation upon specifically transporting its substrate.

2. The fusion protein of claim 1, wherein the fluorescent protein is linked to the N-terminus or C-terminus of the at least one transporter protein.

3. The fusion protein of claim 1 further comprising a fluorescent protein linker peptide that links the at least one fluorescent protein to the at least one transporter protein.

4. The fusion protein of claim 1, wherein the transporter protein is a nitrate transporter, a peptide transporter, or a hormone transporter.

5. The method of claim 1, wherein the transporter protein is a nitrate transporter having an amino acid sequence at least 40% identical to the amino acid sequence of SEQ ID NO:2.

6. The method of claim 1, wherein the transporter protein is a nitrate transporter having an amino acid sequence identical to the amino acid sequence of SEQ ID NO:2.

7. The fusion protein of claim 1, further comprising a second fluorescent protein, wherein the first and second fluorescent proteins emit wavelengths of light that are different from one another.

8. The fusion protein of claim 7, further comprising a second fluorescent protein linker peptide, wherein the first fluorescent protein linker peptide links the first fluorescent protein to the at least one transporter protein and the second fluorescent protein linker peptide links the second fluorescent protein to the at least one transporter protein.

9. The fusion protein of claim 8, wherein the first and second fluorescent protein linker peptides are the same.

10. The fusion protein of claim 8, wherein the first and second fluorescent proteins are selected from the group consisting of green fluorescent protein (GFP), yellow fluorescent protein (YFP), cyan fluorescent protein (CFP), citrine, cerulean, VENUS and teal fluorescent protein (TFP).

11. The fusion protein of claim 1, wherein the transporter protein specifically transports KNO₃.

12. A nucleic acid that encodes the fusion protein of claim 1.

13. A vector comprising the nucleic acid of claim 12.

14. A host cell comprising the vector of claim 13.

15. A plant comprising the host cell of claim 14.

16. A method of producing a fusion protein, the method comprising culturing a host cell in conditions that promote protein expression and recovering fusion protein from the culture, wherein the host cell comprises a vector encoding a fusion protein, wherein the fusion protein comprises at least one fluorescent protein that is linked to at least one transporter protein comprising an N-terminus and a C-terminus, wherein the transporter protein changes three- dimensional conformation upon specifically transporting its substrate.

17. A method of detecting transport of a substrate in a sample, the method comprising contacting the fusion protein of claim 1 with the sample and determining a change in luminescence of the at least one fluorescent protein that occurs after the substrate is transported by the fusion protein.

18. The method of claim 17, wherein the change in luminescence is a change in fluorescence resonance energy transfer (FRET) between the first and second fluorescent proteins that occurs after the substrate is transported by the fusion protein.

19. The method of claim 16, wherein the substrate is KNO₃.

20. The method of claim 16, wherein the sample is in a plant or tissue thereof.

21. The fusion protein of claim 1, wherein the transporter protein is a member of the solute carrier (SLC) group of membrane transporter proteins.

22. The fusion protein of claim 1, wherein the transporter protein is a member of the major facilitator superfamily (MFS).

23. The fusion protein of claim 1, wherein the transporter protein is a hormone transporter having an amino acid sequence at least 40% identical to the amino acid sequence of SEQ ID NO:11 or SEQ ID NO: 14.

24. A fusion protein comprising at least one fluorescent protein that is linked to at least one mechanosensitive ion channel protein comprising an N-terminus and a C-terminus, wherein the mechanosensitive ion channel protein detects esmotic stress.

25. The fusion protein of claim 20, wherein the mechanosensitive ion channel protein is mechanisensitive channel small conductance-like 10 (AtMSL10).
